(19) United States
(12) Patent Application Publication
(10) Pub. No.: US 2024/0197869 A1
(43) Pub. Date: Jun. 20, 2024

(54) HYPERACTIVATORS OF MAMMALIAN DENDRITIC CELLS

(71) Applicant: Corner Therapeutics, Inc., Watertonw, MA (US)

(72) Inventors: Kelsey K. FINN, Cambridge, MA (US); Jonathan CHOW, Newton, MA (US); Emily GOSSELIN, West Roxbury, MA (US); Dania ZHIVAKI, West Roxbury, MA (US)

(73) Assignee: Corner Therapeutics, Inc., Watertown, MA (US)

(21) Appl. No.: 18/286,333

(22) PCT Filed: Apr. 11, 2022
(86) PCT No.: PCT/US2022/071664

§ 371 Date: Oct. 10, 2023

Related U.S. Application Data
(60) Provisional application No. 63/246,740, filed on Sep. 21, 2021.
(60) Provisional application No. 63/173,958, filed on Apr. 12, 2021.

(51) Int. Cl. A61K 39/39; A61K 31/4745; A61K 39/00; A61P 35/00

(52) U.S. Cl. CPC A61K 39/39; A61K 31/4745; A61K 39/0011; A61P 35/00; A61K 2039/55511

(57) ABSTRACT

The present disclosure relates to lysophosphatidylcholine (LPC) compounds and uses thereof in hyperactivating mammalian dendritic cells, such as human dendritic cells or canine dendritic cells. The present disclosure also relates to compositions comprising a LPC and one or more of a pathogen recognition receptor agonist, an antigen, and mammalian dendritic cells, as well as methods for production and use of the compositions.

### CROSS-REFERENCE TO RELATED APPLICATIONS

This application claims benefit of U.S. Provisional Application No. 63/246,740, filed Sep. 21, 2021 and U.S. Provisional Application No. 63/173,958, filed Apr. 12, 2021, the disclosures of which are hereby incorporated by reference in their entirety.

### SUBMISSION OF SEQUENCE LISTING AS ASCII TEXT FILE

None.

### FIELD

The present disclosure relates to lysophosphatidylcholine (LPC) compounds and uses thereof in hyperactivating mammalian dendritic cells, such as human dendritic cells or canine dendritic cells. The present disclosure also relates to compositions comprising a LPC and one or more of a pathogen recognition receptor agonist, an antigen, and human or canine dendritic cells, as well as methods for production and use of the compositions.

### BACKGROUND

Typically, dendritic cell (DC) maturation by vaccine adjuvants such as Toll-like receptor agonists does not lead to IL-1beta secretion. In circumstances such as inflammasome activation, IL-1beta secretion does occur but at the cost of DC death by a lytic process of cell death termed pyroptosis (Evavold et al., J Mol Biol, 430(2):217-237, 2018). However, when DCs are matured using the pathogen-associated molecular pattern (PAMP)-containing molecule, lipopolysaccharide (LPS) and the damage-associated molecular pattern (DAMP)-containing molecule such as PGPC (1-palmitoyl-2-glutaryl-sn-glycero-3-phosphocholine) they produce and secrete IL-1beta without pyroptosing, characterizing these viable DCs as hyperactive (Zanoni et al., Science, 352(6290):1232-1236, 2016). In fact, in mouse models, hyperactivated DCs have demonstrated an improved ability to induce an immune response compared to cells activated using LPS alone (Zhivaki et al., Cell Rep, 33(7):108381, 2020). However, little is known about stimuli effective for hyperactivation of human DCs.

As such, the identification of PAMPs and DAMPs suitable for hyperactivation of human DCs is needed in the art. Additionally, the identification of alternatives to the use of LPS and PGPC for hyperactivation of mammalian DCs is desirable. In particular, while LPS (endotoxin) is a potent PAMP, it is contraindicated for use in humans as it can lead to septic shock.

### BRIEF SUMMARY

The present disclosure relates to lysophosphatidylcholine (LPC) compounds and uses thereof in hyperactivating mammalian dendritic cells, such as human dendritic cells or canine dendritic cells. The present disclosure also relates to compositions comprising a LPC and one or more of a pathogen recognition receptor agonist, an antigen, and human or canine dendritic cells, as well as methods for production and use of the compositions.

In particular, the present disclosure provides a composition comprising an isolated lysophosphatidylcholine (LPC) with a single acyl chain, and a TLR7/8 agonist, wherein the acyl chain is a C13-C22 acyl chain or a C13-C24 acyl chain. In some embodiments, the acyl chain is a C18-C22 acyl chain, a C21-C24 acyl chain, or a C22 acyl chain. In some embodiments, the composition further comprises an antigen and/or dendritic cells.

In some aspects, the present disclosure provides a composition comprising an isolated lysophosphatidylcholine (LPC) with a single acyl chain, and an antigen, wherein the acyl chain is a C21-C24 acyl chain. In some embodiments, the composition further comprises dendritic cells and/or a TLR7/8 agonist.

In some aspects, the present disclosure provides a composition comprising an isolated lysophosphatidylcholine (LPC) with a single acyl chain, and dendritic cells, wherein the acyl chain is a C21-C24 acyl chain. In some embodiments, the composition further comprises a TLR7/8 agonist and/or an antigen.

In some embodiments of the preceding aspects, the antigen is present in a biological sample obtained from an individual. In some embodiments, the biological sample comprises biopsy tissue. In some embodiments, the biological sample comprises cells. In other embodiments, the biological sample does not comprise cells. In some embodiments, the biological sample comprises pus from an abscess. In some embodiments, the antigen comprises a proteinaceous antigen. In some embodiments, the antigen comprises a tumor antigen. In some embodiments, the tumor antigen comprises a synthetic or recombinant neoantigen. In some embodiments, the tumor antigen comprises a tumor cell lysate. In some embodiments, the antigen comprises a microbial antigen and the microbial antigen comprises one or more of a viral antigen, a bacterial antigen, a protozoan antigen, and a fungal antigen. In some embodiments, the microbial antigen comprises a purified or recombinant surface protein. In some embodiments, the microbial antigen comprises an inactivated, whole virus.

In some embodiments, the composition does not comprise liposomes. In some embodiments, the composition does not comprise LPS or MPLA. In some embodiments, the composition does not comprise oxPAPC or a species of oxPAPC. In some embodiments, the composition does not comprise HOdiA-PC, KOdiA-PC, HOOA-PC, KOOA-PC, and/or PGPC.

In some embodiments, the composition further comprises an adjuvant, wherein the adjuvant comprises an aluminum salt adjuvant, a squalene-in-water emulsion, a saponin, or combinations thereof.

In some embodiments, the present disclosure provides a pharmaceutical formulation comprising the composition of any of the preceding aspects and a pharmaceutically acceptable excipient.

In other aspects, the present disclosure provides a method for production of hyperactivated dendritic cells, the method comprising contacting the dendritic cells with a composition comprising effective amounts of an isolated lysophosphatidylcholine (LPC) with a single C13-C22 acyl chain or a C13-C24 acyl chain, and a TLR7/8 agonist for production of hyperactivated dendritic cells, wherein the hyperactivated dendritic cells secrete IL-1beta without undergoing pyroptosis. In some embodiments, the dendritic cells are contacted ex vivo with the composition or pharmaceutical formulation of any one of the preceding embodiments. In other embodiments, the dendritic cells are contacted in vivo with the pharmaceutical formulation comprising the composition of any one of the preceding embodiments. In some aspects, the present disclosure provides a pharmaceutical formulation comprising a plurality of the hyperactivated dendritic cells produced by the preceding embodiments, and a pharmaceutically acceptable excipient. In some embodiments, the plurality comprises at least 10³, 10⁴, 10⁵, 10⁶, 10⁷ or 10⁸ hyperactivated DCs.

In other aspects, the present disclosure provides a composition comprising an isolated lysophosphatidylcholine (LPC) with a single acyl chain, and a pathogen recognition receptor (PRR) agonist, wherein the acyl chain is a C13-C22 acyl chain or a C13-C24 acyl chain. In some embodiments, the PRR agonist is an agonist of a toll-like receptor (TLR), a NOD-like receptor (NLR), a RIG-I-like receptor (RLR), or a C-type lectin receptor (CLR). In some embodiments, the PRR agonist is an agonist of a cytosolic DNA sensor (CDS) or a stimulator of IFN genes (STING). In some embodiments, the PRR agonist comprises a TLR7/8 agonist. In some embodiments, the composition further comprises an antigen and/or dendritic cells.

In some embodiments of the preceding aspects, the acyl chain is a C21-C24 acyl chain. In some embodiments, the acyl chain is a C22 acyl chain. In some embodiments, the acyl chain is fully saturated. In some embodiments, the LPC comprises 1-behenoyl-2-hydroxy-sn-glycero-3-phosphocholine [LPC(22:0)].

In some embodiments of the preceding aspects, the TLR7/8 agonist is a small molecule with a molecule weight of 900 daltons or less. In some embodiments, the TLR7/8 agonist comprises an imidazoquinoline compound. In some embodiments, the TLR7/8 agonist comprises resiquimod (R848). In some embodiments, the LPC comprises LPC(22:0), and the TLR7/8 agonist comprises resiquimod (R848).

The present disclosure further provides compositions for hyperactivation of human dendritic cells, comprising an isolated lysophosphatidylcholine (LPC) compound with a single acyl chain, and a pathogen recognition receptor (PRR) agonist, wherein the acyl chain is C22 acyl chain, and wherein the composition is effective for achieving a higher level of dendritic cell hyperactivation than a comparator composition comprising PGPC in place of the LPC. In some embodiments, the hyperactivation occurs in vitro or ex vivo. In other embodiments, the hyperactivation occurs in vivo. In some embodiments, the higher level of dendritic cell hyperactivation comprises induction of IL-1beta secretion from the human dendritic cells in vitro at a level that is at least 2, 3 or 4 fold higher when contacted with the composition comprising the LPC and the PRR agonist than when contacted with the comparator composition comprising the PGPC and the PRR agonist, wherein the PRR agonist is LPS. In some embodiments, the concentration of the LPC and the concentration of the PGPC are the same concentration, optionally in a range of from about 10 μM to about 80 μM, and the LPS is present at a concentration of 1 μg/ml in both the composition and the comparator composition. In some embodiments, the higher level of dendritic cell hyperactivation comprises a lipid activity index for IL-1beta secretion from the human dendritic cells for the composition comprising the LPC and the PRR agonist that is at least 4, 5 or 6 fold higher in activity units than that of the comparator composition comprising the LPC and the PRR agonist.

### BRIEF DESCRIPTION OF THE DRAWINGS

FIG. 1 is a cartoon showing the effects of various stimuli on dendritic cell function. Depending on the stimuli, dendritic cells remain quiescent, or become activated, pyroptotic, or hyperactivated.

FIG. 2 shows IL-1β secretion by human monocyte-derived dendritic cells activated with 1 μg/mL LPS in the presence of 82.5 μM of various lysophosphatidylcholine compounds (LPCs). The results demonstrate that acyl chain length affects the ability of LPCs to promote IL-1β secretion by DCs.

FIG. 3 shows IL-1β secretion by human monocyte-derived dendritic cells activated with 1 μg/mL LPS in the presence of 82.5 μM of various lipids. The results demonstrate that lysophosphatidylcholine compounds (LPCs) with an acyl chain length of at least 12 carbons (12:0 Lyso PC, No. 29) are capable of stimulating human DCs to secrete IL-1β at levels comparable to or better (22:0 Lyso PC, No. 42) than DAMPs such as PGPC, which had been identified by screening murine DCs.

FIG. 4 shows a lipid activity index of various compounds. The index was calculated by multiplying the reciprocal of the lowest concentration at which IL-1β secretion by human monocyte-derived dendritic cells was 2-fold higher than the LPS only control by the highest IL-1β signal observed at any concentration.

FIG. 5 shows IL-1β secretion by human monocyte-derived dendritic cells contacted with various PRR agonists in the presence or absence of 22:0 Lyso PC.

FIG. 6A-6B show IL-1β secretion by canine peripheral blood mononuclear cells (PBMCs) two days post-activation with the indicated stimuli, shown as total concentration (FIG. 6A) and fold change per donor relative to R848 alone (FIG. 6B), respectively. The results demonstrate that 22:0 LYSO PC, when combined with R848, is capable of stimulating canine PBMCs to secrete IL-1β at levels comparable or higher than DAMPs such as PGPC or LPS and Alum. FIG. 6C shows the relative viability of canine PBMCs two days post-activation with the indicated stimuli. The results demonstrate that canine PBMCs remain viable after treatment with 22:0 LYSO PC.

FIG. 7A-7B show IL-1β secretion by human PBMCs two days post-activation with the indicated stimuli, shown as total concentration (FIG. 7A) and fold change per donor relative to R848 alone (FIG. 7B). The results demonstrate that 22:0 LYSO PC, when combined with R848, is capable of stimulating human PBMCs to secrete IL-1β at levels comparable or higher than DAMPs such as PGPC or LPS and Alum. FIG. 7C shows the relative viability of human PBMCs two days post-activation with the indicated stimuli. The results demonstrate that human PBMCs remain viable after treatment with 22:0 LYSO PC.

FIG. 8A-8B show IFNγ (FIG. 8A) and TNFα (FIG. 8B) secretion by human PBMCs two days-post activation with the indicated stimuli, shown as fold change per donor relative to R848 alone. The results demonstrate that 22:0 LYSO PC, when combined with R848, is capable of stimulating human PBMCs to secrete other immunostimulatory cytokines at levels comparable or higher than DAMPs such as PGPC or LPS and Alum.

FIG. 9A-9B show viability (FIG. 9A) and IL-1β secretion (FIG. 9B) by non-human primate, monocyte-derived dendritic cells (moDC) under various activation conditions (unstimulated, or contacted with R848 in the presence or absence of 22:0 Lyso PC or PGPC).

FIG. 10A-10B show IL-1β secretion by non-human primate PBMCs two days post-activation with the indicated stimuli, shown as total concentration (FIG. 10A) and fold change relative to R848 alone (FIG. 10B).

FIG. 11A-11B show IFN-γ secretion by non-human primate PBMCs two days post-activation with the indicated stimuli, shown as total concentration (FIG. 11A) and fold change relative to R848 alone (FIG. 11B).

FIG. 12A-12B show IL-17a secretion by non-human primate PBMCs two days post-activation with the indicated stimuli, shown as total concentration (FIG. 12A) and fold change relative to R848 alone (FIG. 12B).

FIG. 13A-13B show IL-23 secretion by non-human primate PBMCs two days post-activation with the indicated stimuli, shown as total concentration (FIG. 13A) and fold change relative to R848 alone (FIG. 13B).

FIG. 14A-14B show IFN-β secretion by non-human primate PBMCs two days post-activation with the indicated stimuli, shown as total concentration (FIG. 14A) and fold change relative to R848 alone (FIG. 14B).

FIG. 15A-15B show IL-8 secretion by non-human primate PBMCs two days post-activation with the indicated stimuli, shown as total concentration (FIG. 15A) and fold change relative to R848 alone (FIG. 15B).

FIG. 16A-16B show IL-6 secretion by non-human primate PBMCs two days post-activation with the indicated stimuli, shown as total concentration (FIG. 16A) and fold change relative to R848 alone (FIG. 16B).

FIG. 17 shows IFN-γ secretion by human memory CD4+ T cells.

FIG. 18A-18B show IL-4 (FIG. 18A) and IL-13 (FIG. 18B) secretion by human memory CD4+ T cells.

FIG. 19A-19C show IL-17a (FIG. 19A), IL-17f (FIG. 19B) and IL-22 (FIG. 19C) by human memory CD4+ T cells.

FIG. 20 shows Th1 polarization of human naïve CD4+ T cells as a consequence of co-culture with moDC treated with R848 and 22:0 LYSO PC, relative to co-culture with moDC treated with R848 alone.

FIG. 21 shows viability of human moDC cultured in the presence of PBS or various filtered or unfiltered lipid formulations.

FIG. 22 shows IL-1β secretion by human moDC cultured in the presence of PBS or various filtered or unfiltered lipid formulations.

FIG. 23A-23B show IL-1β secretion (FIG. 23A) by and viability (FIG. 23B) of human moDC cultured in the presence of PBS or various filtered formulations.

FIG. 24 shows the characterization of 22:0 LYSO PC-containing particle sizes as determined by dynamic light scattering.

FIG. 25A-25B show IL-1β secretion (FIG. 25A) by and viability (FIG. 25B) of murine, FLT3L-differentiated DC cultured under the indicated conditions.

FIG. 26A-26C show TNF-alpha (FIG. 26A), IL-6 (FIG. 26B) and IL-12p40 (FIG. 26C) by murine, FLT3L-differentiated DC cultured under the indicated conditions.

FIG. 27 shows co-stimulatory molecule (CD40) expression by murine, FLT3L-differentiated cDC1 and cDC2 cells cultured under the indicated conditions. Means and SDs from at least two replicates are shown, and data are representative of at least two independent experiments. P values of <0.05 (*), <0.01 (**) or <0.001(***), %0.0001 (****) indicated significant differences between groups. TWO-way ANOVA test was used.

FIG. 28A-28B show CCR7 (FIG. 28A) and CXCL16 (FIG. 28B) expression by murine, FLT3L-differentiated cDC1 and cDC2 cells cultured under the indicated conditions.

FIG. 29 shows MHC class I expression by murine, FLT3L-differentiated cDC1 and cDC2 cells cultured under the indicated conditions. Means and SDs from at least two replicates are shown, and data are representative of at least two independent experiments. P values of <0.05 (*), <0.01 (**) or <0.001(***), %0.0001 (****) indicated significant differences between groups. TWO-way ANOVA test was used.

FIG. 30A-30B show antigen uptake (FIG. 30A) and antigen presentation (FIG. 30B) by murine, FLT3L-differentiated DC cultured under the indicated conditions. Antigen uptake was assessed by measuring endocytosis of Red pHrodo dextran. Antigen presentation was assessed by measuring ovalbumin peptide bound to MHC class I, H-2Kb.

FIG. 31 shows DC infiltration of draining lymph nodes (dLN) of the skin of mice after subcutaneous injection of the indicated formulations comprising R848, 22:0 LYSO PC, and the surfactant, KP407.

FIG. 32 shows survival of tumor-bearing mice treated with PBS or a therapeutic cancer vaccine (e.g., a whole tumor lysate formulation).

FIG. 33 shows kinetics of tumor growth of mice treated with PBS or a therapeutic cancer vaccine (e.g., a whole tumor lysate formulation).

### DETAILED DESCRIPTION

The present disclosure relates to lysophosphatidylcholine (LPC) compounds and uses thereof in hyperactivating human dendritic cells. The present disclosure also relates to compositions comprising a LPC and one or more of a pathogen recognition receptor agonist, an antigen, and human dendritic cells, as well as methods for production and use of the compositions. In further embodiments, the dendritic cells are non-human dendritic cells, with the proviso that the dendritic cells are not rodent dendritic cells.

### General Techniques and Definitions

The practice of the present disclosure will employ, unless otherwise indicated, conventional techniques of molecular biology (including recombinant techniques), microbiology, cell biology, biochemistry and immunology, which are within the skill of the art.

As used herein and in the appended claims, the singular forms “a”, “an”, and “the” include plural references unless indicated otherwise. For example, “an” excipient includes one or more excipients.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments.

The term “about” as used herein in reference to a value, encompasses from 90% to 110% of that value (e.g., a molecular weight of about 900 daltons, refers to a molecular weight of from 910 daltons to 990 daltons).

An “effective amount” or a “sufficient amount” of a substance is that amount sufficient to effect beneficial or desired results, including clinical results, and, as such, an “effective amount” depends upon the context in which it is being applied. For instance, in the context of administering an immunogenic composition, an effective amount contains sufficient antigen, and one or both of a lysophosphatidylcholine (LPC) compound and a PRR agonist, to stimulate an immune response against the antigen (e.g., antigen-reactive antibody and/or cellular immune response).

The terms “individual” and “subject” refer to mammals. “Mammals” include, but are not limited to, humans, non-human primates (e.g., monkeys), farm animals, sport animals, rodents (e.g., mice and rats), and pets (e.g., dogs and cats). In some embodiments, the subject is a human patient, such as a human patient suffering from cancer and/or an infectious disease.

The term “dose” as used herein in reference to an immunogenic composition refers to a measured portion of the immunogenic composition taken by (administered to or received by) a subject at any one time.

The terms “isolated” and “purified” as used herein refers to a material that is removed from at least one component with which it is naturally associated (e.g., removed from its original environment). As an example, when used in reference to an LPC, an isolated LPC is at least 90%, 95%, 96%, 97%, 98% or 99% pure as determined by thin layer chromatography, or gas chromatography. As a further example, when used in reference to a recombinant protein, an isolated protein refers to a protein that has been removed from the culture medium of the host cell that produced the protein.

The terms “pharmaceutical formulation” and “pharmaceutical composition” refer to preparations that are in such form as to permit the biological activity of the active ingredient to be effective, and that contain no additional components that are unacceptably toxic to an individual to which the formulation or composition would be administered. Such formulations or compositions are intended to be sterile.

“Excipients” as used herein include pharmaceutically acceptable excipients, carriers, vehicles or stabilizers that are nontoxic to the cell or mammal being exposed thereto at the dosages and concentrations employed. Often the physiologically acceptable excipient is an aqueous pH buffered solution.

The term “antigen” refers to a substance that is recognized and bound specifically by an antibody or by a T cell antigen receptor. Antigens can include peptides, polypeptides, proteins, glycoproteins, polysaccharides, complex carbohydrates, sugars, gangliosides, lipids and phospholipids; portions thereof and combinations thereof. Antigens when present in the compositions of the present disclosure can be synthetic or isolated from nature. Antigens suitable for administration in the methods of the present disclosure include any molecule capable of eliciting an antigen-specific B cell or T cell response. Haptens are included within the scope of “antigen.” A “hapten” is a low molecular weight compound that is not immunogenic by itself but is rendered immunogenic when conjugated with a generally larger immunogenic molecule (carrier).

“Polypeptide antigens” can include purified native peptides, synthetic peptides, recombinant peptides, crude peptide extracts, or peptides in a partially purified or unpurified active state (such as peptides that are part of attenuated or inactivated viruses, microorganisms or cells), or fragments of such peptides. Polypeptide antigens are preferably at least eight amino acid residues in length.

The term “agonist” is used in the broadest sense and includes any molecule that activates signaling through a receptor. In some embodiments, the agonist binds to the receptor. For instance, a TLR8 agonist binds to a TLR8 receptor and activates a TLR8-signaling pathway.

“Alkyl” refers to monovalent saturated aliphatic hydrocarbyl groups. Cx alkyl refers to an alkyl group having x number of carbon atoms. Cx-Cy alkyl or Cx-y alkyl refers to an alkyl group having between x number and y number of carbon atoms, inclusive.

“Alkylene” refers to divalent saturated aliphatic hydrocarbyl groups.

“Alkenyl” refers to monovalent hydrocarbyl groups having at least one double bond (>C═C<). Cx alkenyl refers to an alkenyl group having x number of carbon atoms. Cx-Cy alkenyl or Cx-y alkenyl refers to an alkenyl group having between x number and y number of carbon atoms, inclusive.

“Stimulation” of a response or parameter includes eliciting and/or enhancing that response or parameter when compared to otherwise same conditions except for a parameter of interest, or alternatively, as compared to another condition (e.g., increase in TLR-signaling in the presence of a TLR agonist as compared to the absence of the TLR agonist). For example, “stimulation” of an immune response means an increase in the response. Depending upon the parameter measured, the increase may be from 2-fold to 2,000-fold, or from 5-fold to 500-fold or over, or from 2, 5, 10, 50, or 100-fold to 500, 1,000, 2,000, 5,000, or 10,000-fold.

Conversely, “inhibition” of a response or parameter includes reducing and/or repressing that response or parameter when compared to otherwise same conditions except for a parameter of interest, or alternatively, as compared to another condition (e.g., decrease in abnormal cell proliferation after administration of a composition comprising a LPC compound and one or more of a pathogen recognition receptor agonist, an antigen, and human dendritic cells, as compared to the administration of a placebo composition or no treatment). For example, “inhibition” of an immune response means a decrease in the response. Depending upon the parameter measured, the decrease may be from 2-fold to 2,000-fold, or from 5-fold to 500-fold or over, or from 2, 5, 10, 50, or 100-fold to 500, 1,000, 2,000, 5,000, or 10,000-fold.

The relative terms “higher” and “lower” refer to a measurable increase or decrease, respectively, in a response or parameter when compared to otherwise same conditions except for a parameter of interest, or alternatively, as compared to another condition. For instance, a “higher level of DC hyperactivation” refers to a level of DC hyperactivation as a consequence of a treatment condition (comprising a LPC compound of the present disclosure) that is at least 2, 3, 4, 5, 6, 7, 8, 9, or 10-fold above a level of DC hyperactivation as a consequence of a control condition (e.g., no LPC, PGPC, oxPAPC, etc.). Likewise, a “lower level of DC hyperactivation” refers to a level of DC hyperactivation as a consequence of a treatment condition (comprising a LPC compound of the present disclosure) that is at least 2, 3, 4, 5, 6, 7, 8, 9, or 10-fold below a level of DC hyperactivation as a consequence of a control condition (e.g., no LPC, PGPC, oxPAPC, etc.).

As used herein the term “immunization” refers to a process that increases a mammalian subject's reaction to antigen and therefore improves its ability to resist or overcome infection and/or resist disease.

The term “vaccination” as used herein refers to the introduction of vaccine into a body of a mammalian subject.

“Adjuvant” refers to a substance which, when added to a composition comprising an antigen, enhances or potentiates an immune response to the antigen in the mammalian recipient upon exposure.

The terms “treating” or “treatment” of a disease refer to executing a protocol, which may include administering one or more therapeutic agents to an individual (human or otherwise), in an effort to obtain beneficial or desired results in the individual, including clinical results. Beneficial or desired clinical results include, but are not limited to, alleviation or amelioration of one or more signs or symptoms of a disease, diminishment of extent of disease, stabilized (i.e., not worsening) state of disease, preventing spread of disease, delay or slowing of disease progression, amelioration or palliation of the disease state, and remission (whether partial or total). “Treatment” also can mean prolonging survival as compared to expected survival of an individual not receiving treatment. Further, “treating” and “treatment” may occur by administration of one dose of a therapeutic agent or therapeutic agents, or may occur upon administration of a series of doses of a therapeutic agent or therapeutic agents. “Treating” or “treatment” does not require complete alleviation of signs or symptoms, and does not require a cure, and specifically includes protocols that have only a palliative effect on the individual. “Palliating” a disease or disorder means that the extent and/or undesirable clinical manifestations of the disease or disorder are lessened and/or time course of progression of the disease or disorder is slowed, as compared to the expected untreated outcome.

### I. Lysophosphatidylcholine Compounds

A “lysophosphatidylcholine” (LPC) or “lysophosphatidylcholine molecule” refers to a glycerol molecule bearing one phosphocholine group on a hydroxyl group of the glycerol and bearing one acyl group on one of the other two hydroxyl groups of the glycerol. The remaining hydroxyl group is unsubstituted.

In some embodiments, the isolated lysophosphatidylcholine (LPC) with a single acyl chain is of the form:

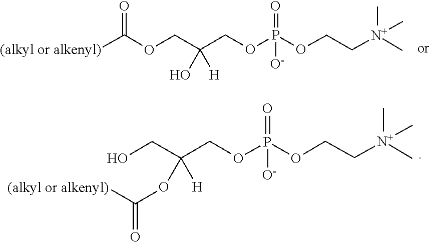

In some embodiments, the isolated lysophosphatidylcholine (LPC) with a single acyl chain is of the form:

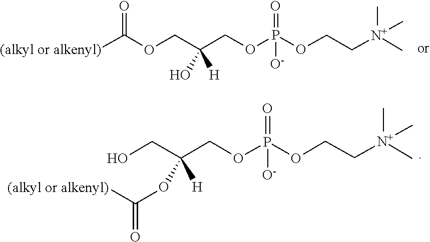

The alkyl or alkenyl chain, together with the carbonyl carbon, forms an acyl chain which is one carbon atom longer than the alkyl or alkenyl chain. For example, a (C23 alkyl)-C(═O)— group forms a C24 acyl chain. Thus, when the group “(alkyl or alkylene)” is a C12-C23 alkyl group (such as a C12-C19 alkyl group or a C20-C23 alkyl group), the (C12-C23 alkyl-C(═O)— group forms a C13-C24 acyl chain (such as a C13-C20 acyl chain or a C21-C24 acyl chain). When the group “(alkyl or alkylene)” is a C12-C23 alkenyl group (such as a C12-C19 alkenyl group or a C20-C23 alkenyl group), the (C12-C23 alkenyl-C(═O)— group forms a C13-C24 acyl chain (such as a C13-C20 acyl chain or a C21-C24 acyl chain). Acyl chains can be referred to as saturated acyl or unsaturated acyl to distinguish between alkyl-containing and alkenyl-containing acyl groups. Standard delta notation or omega notation can be used to indicate the position of one or more double bonds in an unsaturated acyl chain.

Lysophosphatidylcholine (LPC) compounds of the present disclosure have a single acyl chain in which the acyl chain is a C13-C22 acyl chain or a C13-C24 acyl chain. In some embodiments, the acyl chain is a C18-C22 acyl chain or a C21-C24 acyl chain. In some embodiments, the acyl chain is a C21 acyl chain or a C22 acyl chain. In some preferred embodiments, the acyl chain is a C22 acyl chain. Names and structures of exemplary LPC compounds of the present disclosure, as well as their Chemical Abstract Service (CAS) Registry Numbers are shown in Table I as #s 30-43, optionally #s 30-42.

TABLE I
Lipid DAMPS

| Compound | Structure |
|---|---|
| 13:0 Lyso PC #30 | 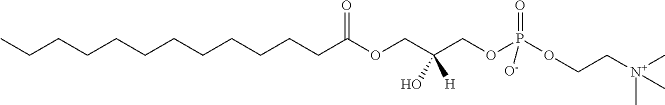 [SMILES: CCCCCCCCCCCCC(=O)OC[C@@H](O)COP(=O)([O-])OCC[N+](C)(C)C] |
| | 1-tridecanoyl-2-hydroxy-sn-glycero-3-phosphocholine |
| | CAS 20559-17-5 |
| 14:0 Lyso PC #31 | 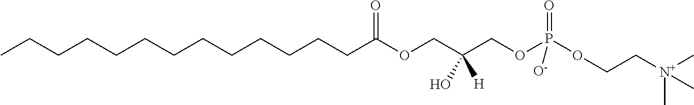 [SMILES: CCCCCCCCCCCCCC(=O)OC[C@@H](O)COP(=O)([O-])OCC[N+](C)(C)C] |
| | 1-myristoyl-2-hydroxy-sn-glycero-3-phosphocholine |
| | CAS 20559-16-4 |
| 15:0 Lyso PC #32 | 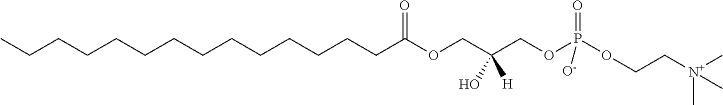 [SMILES: CCCCCCCCCCCCCCC(=O)OC[C@@H](O)COP(=O)([O-])OCC[N+](C)(C)C] |
| | 1-pentadecanoyl-2-hydroxy-sn-glycero-3-phosphocholine |
| | CAS 108273-89-8 |
| 2-16:0 Lyso PC #33 | 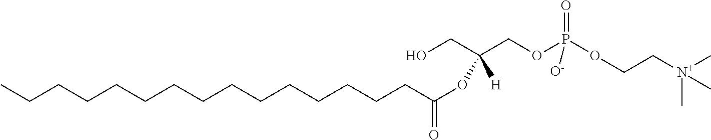 [SMILES: CCCCCCCCCCCCCCCC(=O)O[C@H](CO)COP(=O)([O-])OCC[N+](C)(C)C] |
| | 1-hydroxy-2-palmitoyl-sn-glycero-3-phosphocholine |
| | CAS 66757-27-5 |
| 16:0 Lyso PC #34 | 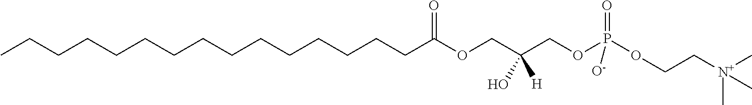 [SMILES: CCCCCCCCCCCCCCCC(=O)OC[C@@H](O)COP(=O)([O-])OCC[N+](C)(C)C] |
| | 1-palmitoyl-2-hydroxy-sn-glycero-3-phosphocholine |
| | CAS 17364-16-8 |
| 17:0 Lyso PC #35 | 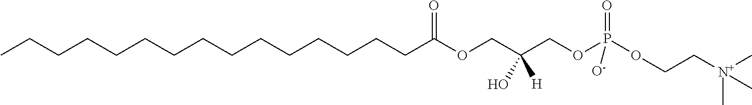 [SMILES: CCCCCCCCCCCCCCCC(=O)OC[C@@H](O)COP(=O)([O-])OCC[N+](C)(C)C] |
| | 1-heptadecanoyl-2-hydroxy-sn-glycero-3-phosphocholine |
| | CAS 50930-23-9 |
| 17:1 Lyso PC #36 | 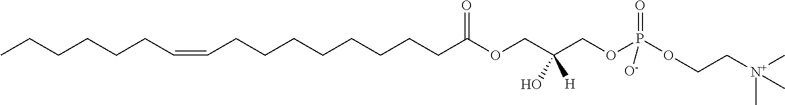 [SMILES: CCCCCC/C=C\CCCCCCCCC(=O)OC[C@@H](O)COP(=O)([O-])OCC[N+](C)(C)C] |
| | 1-(10Z-heptadecenoyl)-2-hydroxy-sn-glycero-3-phosphocholine |
| | CAS 1246304-62-0 |
| 2-18:1 Lyso PC #37 | 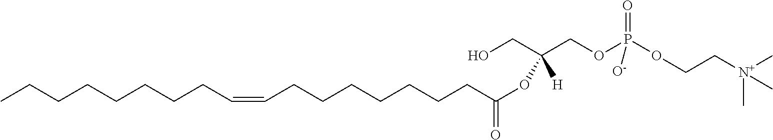 [SMILES: CCCCCCCC/C=C\CCCCCCCC(=O)O[C@H](CO)COP(=O)([O-])OCC[N+](C)(C)C] |
| | 1-hydroxy-2-oleoyl-sn-glycero-3-phosphocholine |
| | CAS 22248-65-3 |
| 2-18:0 Lyso PC #38 | 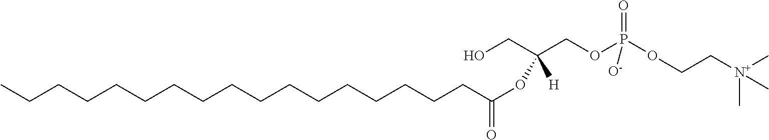 [SMILES: CCCCCCCCCCCCCCCCCC(=O)O[C@H](CO)COP(=O)([O-])OCC[N+](C)(C)C] |
| | 2-stearoyl-sn-glycero-3-phosphocholine |
| | CAS 4421-58-3 |
| 18:0 Lyso PC #39 | 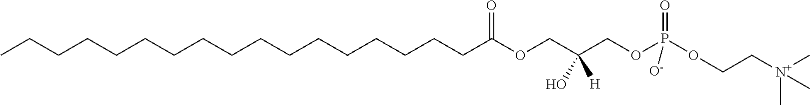 [SMILES: CCCCCCCCCCCCCCCCCC(=O)OC[C@@H](O)COP(=O)([O-])OCC[N+](C)(C)C] |
| | 1-stearoyl-2-hydroxy-sn-glycero-3-phosphocholine |
| | CAS 19420-57-6 |
| 19:0 Lyso PC #40 | 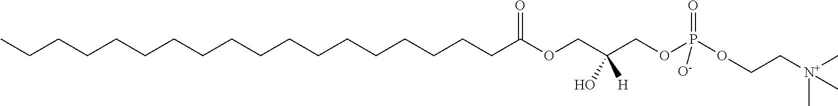 [SMILES: CCCCCCCCCCCCCCCCCCC(=O)OC[C@@H](O)COP(=O)([O-])OCC[N+](C)(C)C] |
| | 1-nonadecanoyl-2-hydroxy-sn-glycero-3-phosphocholine |
| | CAS 108273-88-7 |
| 20:0 Lyso PC #41 | 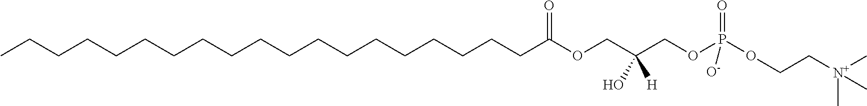 [SMILES: CCCCCCCCCCCCCCCCCCCC(=O)OC[C@@H](O)COP(=O)([O-])OCC[N+](C)(C)C] |
| | 1-arachidoyl-2-hydroxy-sn-glycero-3-phosphocholine |
| | CAS 108341-80-6 |
| 22:0 Lyso PC #42 | 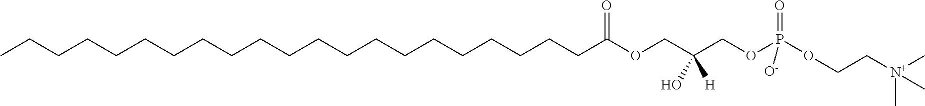 [SMILES: CCCCCCCCCCCCCCCCCCCCCC(=O)OC[C@@H](O)COP(=O)([O-])OCC[N+](C)(C)C] |
| | 1-behenoyl-2-hydroxy-sn-glycero-3-phosphocholine |
| | CAS 125146-65-8 |
| 24:0 Lyso PC #43 | 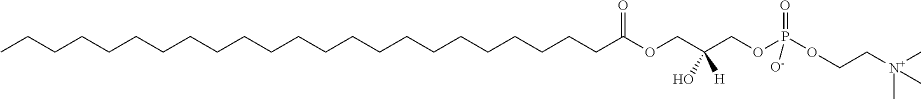 [SMILES: CCCCCCCCCCCCCCCCCCCCCCCC(=O)OC[C@@H](O)COP(=O)([O-])OCC[N+](C)(C)C] |
| | 1-lignoceroyl-2-hydroxy-sn-glycero-3-phosphocholine |
| | CAS 325171-59-3 |
| PGPC #45 | 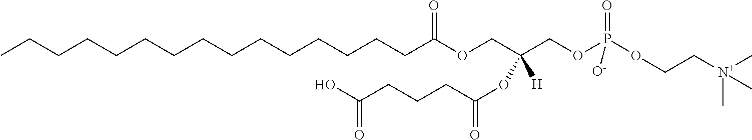 [SMILES: CCCCCCCCCCCCCCCC(=O)OC[C@H](COP(=O)([O-])OCC[N+](C)(C)C)OC(=O)CCCC(=O)O] |
| | 1-palmitoyl-2-glutaryl-sn-glycero-3-phosphocholine |
| | CAS 89947-79-5 |
| POVPC #46 | 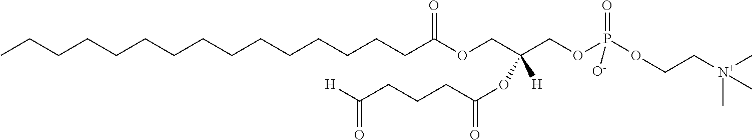 [SMILES: CCCCCCCCCCCCCCCC(=O)OC[C@H](COP(=O)([O-])OCC[N+](C)(C)C)OC(=O)CCCC=O] |
| | 1-palmitoyl-2-(5′-oxo-valeroyl)-sn-glycero-3-phosphocholine |
| | CAS 121324-31-0 |
| PAPC | 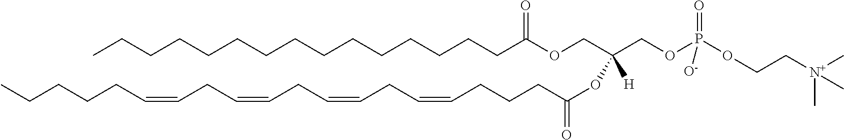 [SMILES: CCCCC/C=C\C/C=C\C/C=C\C/C=C\CCCC(=O)O[C@H](COC(=O)CCCCCCCCCCCCCCC)COP(=O)([O-])OCC[N+](C)(C)C] |
| | 16:0-20:4 PC |
| | 1-palmitoyl-2-arachidonoyl-sn-glycero-3-phosphocholine |
| | CAS 35418-58-7 |
| oxPAPC | Oxidized 1-palmitoyl-2-arachidonoyl-sn-glycero-3-phosphocholine |
| #47 | e.g., Product 870604 from Avanti® Polar Lipids |
| PAzePC #48 | 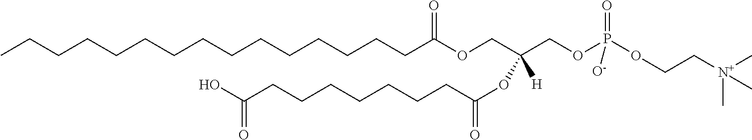 [SMILES: CCCCCCCCCCCCCCCC(=O)OC[C@H](COP(=O)([O-])OCC[N+](C)(C)C)OC(=O)CCCCCCCC(=O)O] |
| | 1-palmitoyl-2-azelaoyl-sn-glycero-3-phosphocholine |
| | CAS 117746-89-1 |

### II. Pathogen Recognition Receptor Agonists

Compositions and methods of the present disclosure may further comprise a pathogen recognition receptor (PRR) agonist. In some embodiments, the PRR agonist comprises an agonist of a toll-like receptor (TLR), a NOD-like receptor (NLR), a RIG-I-like receptor (RLR), or a C-type lectin receptor (CLR). In other embodiments, the PRR agonist comprises a cytosolic DNA sensor (CDS) or a stimulator of IFN genes (STING). In some embodiments, the PRR agonist comprises a TLR7/8 agonist.

### A. TLR7/8 Agonists

The term “TLR7/8 agonist” as used herein refers to an agonist of TLR7 and/or TLR8. In one aspect, the TLR7/8 agonist is a TLR7 agonist. In another aspect, the TLR7/8 agonist is a TLR8 agonist. In a further aspect, the TLR7/8 agonist is an agonist of both TLR7 and TLR8. TLR7/8 agonists of the present disclosure are suitable for hyperactivating human dendritic cells in the presence of LPC.

In some aspects, the TLR7/8 agonist is a small molecule. In some embodiments, the TLR7/8 agonist is a small molecule with a molecule weight of 900 daltons or less, or a salt thereof. That is, the small molecule TLR7/8 agonist is not a large molecule like a recombinant protein or a synthetic oligonucleotide, which is regulatable by the U.S. FDA's Center for Biologics Evaluation and Research. Rather the small molecule TLR7/8 agonist is regulatable by the FDA's Center for Drug Evaluation and Research. In some embodiments, the small molecule has a molecule weight of from about 90 to about 900 daltons. In some embodiments, the TLR7/8 agonist comprises an imidazoquinoline compound. In some preferred embodiments, the TLR7/8 agonist comprises resiquimod (R848).

### B. Other PRR Agonists

In some aspects, the pathogen recognition receptor (PRR) agonist comprises a toll-like receptor (TLR) agonist with the proviso that the TLR agonist does not comprise a TLR7/8 agonist. In some embodiments, the TLR agonist comprises an agonist of one or more of TLR2, TLR3, TLR4, TLR5, TLR9 and TLR13. In some embodiments, the PRR agonist is a TLR2/6 agonist, such as Pam2CSK4. In other embodiments, the TLR agonist is a TLR4 agonist such as monophosphoryl lipid A (MPLA). However, in preferred embodiments, the TLR agonist is not an agonist of TLR2, TLR4 and/or TLR9. For instance, in preferred embodiments, the TLR9 agonist is not a TLR4 ligand such as LPS (endotoxin).

In other aspects, the PRR agonist comprises a NOD-like receptor (NLR) agonist. In further aspects, the PRR agonist comprises a RIG-I-like receptor (RLR) agonist. In additional aspects, the PRR agonist comprises a C-type lectin receptor (CLR) agonist. In still further aspects, the PRR agonist comprises a CDS agonist or a STING agonist.

### III. Antigens

Compositions and methods of the present disclosure may further comprise an antigen. In some embodiments, the antigen comprises a proteinaceous antigen. The terms “polypeptide” and “protein” are used interchangeably herein to refer to proteinaceous antigens that comprise peptide chains that are at least 8 amino acids in length. In some embodiments, the proteinaceous antigen is from 8 to 1800 amino acids, 9 to 1000 amino acids, or 10 to 100 amino acids in length. In some embodiments, the antigen comprises a synthetic protein or a recombinant protein. In other embodiments, the antigen comprises a protein purified from a biological sample. The polypeptide may be post-translationally modified such as by phosphorylation, hydroxylation, sulfonation, palmitoylation, and/or glycosylation.

In some embodiments, the antigen is a tumor antigen that comprises the amino acid sequence of at least one full length protein or fragment thereof. In some embodiments, the tumor antigen comprises an amino acid sequence or fragment thereof from an oncoprotein. In some embodiments, the mammalian antigen is a neoantigen or encoded by a gene comprising a mutation relative to the gene present in normal cells from a mammalian subject. Neoantigens are thought to be particularly useful in enabling T cells to distinguish between cancer cells and non-cancer cells (see, e.g., Schumacher and Schreiber, Science, 348:69-74, 2015). In other embodiments, the tumor antigen comprises a viral antigen, such as an antigen of a cancer-causing virus.

In some embodiments, the tumor antigen is a fusion protein comprising two or more polypeptides, wherein each polypeptide comprises an amino acid sequence from a different tumor antigen or non-contiguous amino acid sequences from the same tumor antigen. In some of these embodiments, the fusion protein comprises a first polypeptide and a second polypeptide, wherein each polypeptide comprises non-contiguous amino acid sequences from the same tumor antigen.

In some embodiments, the antigen is a microbial antigen. In some embodiments, the microbial antigen comprises a viral antigen, a bacterial antigen, a protozoan antigen, a fungal antigen, or combinations thereof. In some embodiments, the microbial antigen comprises a surface protein or other antigenic subunit of a microbe. In other embodiments, the microbial antigen comprises an inactivated or attenuated microbe. For instance, the microbial antigen may comprise an inactivated virus, such as a chemically or genetically-inactivated virus. Alternatively, the microbial antigen may comprise a virus-like particle.

In some embodiments, the antigen may be present in a biological sample obtained from an individual, such as a human patient. For instance, the antigen may comprise cancer cells. In another aspect, the antigen may comprise microbially-infected cells, such as virally-infected cells.

### IV. Dendritic Cells

Compositions and methods of the present disclosure may further comprise dendritic cells (DCs), which are antigen presenting cells that are thought to bridge the innate and adaptive immune systems of mammals. In preferred embodiments, the DCs are subset-1 conventional DCs (cDCIs, previously referred to as myeloid DCIs), as opposed to plasmacytoid DCs (pDCs).

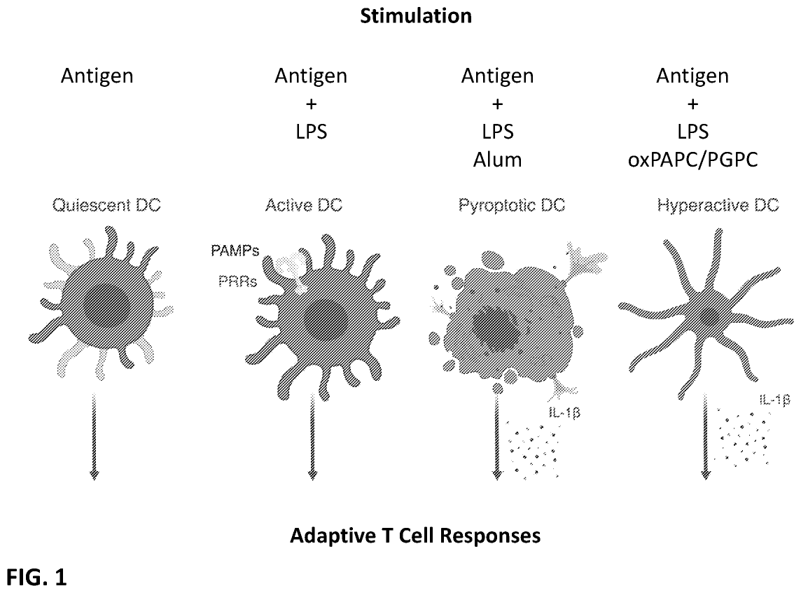

In some embodiments, the DCs are hyperactive DCs that express high levels of CD40 and IL-12p70. As used herein, the term “hyperactive dendritic cells” refer to a cell state in which DCs are able to secrete IL-1β while maintaining cellular viability (e.g., without undergoing pyroptosis). In this way, hyperactivated dendritic cells are able to stimulate robust T cell immunity (FIG. 1), which apparently combines the benefits of activated and pyroptotic dendritic cells (Zhivaki et al., Cell Reports, 33 (7), 2020, 108381).

### V. Pharmaceutical Formulations

Some compositions of the present disclosure are pharmaceutical formulations comprising a pharmaceutically acceptable excipient, and an LPC compound, a PRR agonist, a dendritic cell, an antigen, an adjuvant, or any combination thereof. Pharmaceutical formulations of the present disclosure may be in the form of a solution or a suspension. Alternatively, the pharmaceutical formulations may be a dehydrated solid (e.g., freeze dried or spray dried solid). The pharmaceutical formulations of the present disclosure are preferably sterile, and preferably essentially endotoxin-free. The term “pharmaceutical formulations” is used interchangeably herein with the terms “medicinal product” and “medicament”. In some embodiments, the pharmaceutical formation comprises specific ratios of the various components based on the intended purpose of the formulation. In some embodiments, the pharmaceutical formulations comprise an LPC compound and non-ionic surfactant. In some embodiments, the non-ionic surfactant comprises an ethylene oxide-propylene oxide copolymer, such as Poloxamer-407 (CAS Registry No. 977057-91-2).

### A. Excipients

Pharmaceutically acceptable excipients of the present disclosure include for instance, solvents, buffering agents, tonicity adjusting agents, bulking agents, and preservatives (See, e.g., Pramanick et al., Pharma Times, 45:65-77, 2013). In some embodiments, the pharmaceutical formulations may comprise an excipient that functions as one or more of a solvent, a buffering agent, a tonicity adjusting agent, and a bulking agent (e.g., sodium chloride in saline may serve as both an aqueous vehicle and a tonicity adjusting agent). Pharmaceutically acceptable excipients of the present disclosure also include detergents, wetting agents, emulsifiers, foaming agents, and dispersants, as well as surfactants.

Many of the lipids disclosed herein are sparingly soluble in water. Surfactants can be used to solubilize the lipids in aqueous formulations. A wide variety of surfactants are available, which can be classified as anionic surfactants, non-ionic surfactants, cationic surfactants, and zwitterionic surfactants.

Some examples of non-ionic surfactants include poloxamers, which are triblock copolymers of ethylene oxide and propylene oxide of the general formula: HO—[CH₂CH₂—O-]ₐ-[CH₂CH(CH₃)—O-]_{b}-[CH₂—CH₂—O-]ₐ-H. Some poloxamers are sold under the trade name Pluronic® (PLURONIC is a registered trademark of BASF SE, Ludwigshafen, Germany). Examples of poloxamers are Poloxamer 407 (KP407; a=101, b=56); Poloxamer 188 (KP188; a=80, b=27); Pluronic® P84 (P-84; a=19, b=39); and Pluronic® P123 (P-123; a=20, b=70) (the foregoing values for a and b may be subject to slight variation).

Other non-ionic surfactants include the Cremophor® series (CREMAPHOR is a registered trademark of BASF SE, Ludwigshafen, Germany). Cremophor® surfactants include Cremophor® EL (K EL), a mixture of polyoxyethylated triglycerides produced by reacting castor oil with ethylene oxide in a molar ratio of approximately 1:35, and Cremophor® RH40 (also known as Kolliphor® RH40; KOLLIPHOR is a registered trademark of BASF SE), obtained by reacting 40 moles of ethylene oxide with 1 mole of hydrogenated castor oil.

In some embodiments, the pharmaceutical formulations comprise an aqueous vehicle as a solvent. Suitable vehicles include for instance sterile water, saline solution, phosphate buffered saline, and Ringer's solution. In some embodiments, the composition is isotonic.

The pharmaceutical formulations may comprise a buffering agent. Buffering agents control pH to inhibit degradation of the active agent during processing, storage and optionally reconstitution. Suitable buffers include for instance salts comprising acetate, citrate, phosphate or sulfate. Other suitable buffers include for instance amino acids such as arginine, glycine, histidine, and lysine. The buffering agent may further comprise hydrochloric acid or sodium hydroxide. In some embodiments, the buffering agent maintains the pH of the composition within a range of 6 to 9. In some embodiments, the pH is greater than (lower limit) 6, 7 or 8. In some embodiments, the pH is less than (upper limit) 9, 8, or 7. That is, the pH is in the range of from about 6 to 9 in which the lower limit is less than the upper limit.

The pharmaceutical compositions may comprise a tonicity adjusting agent. Suitable tonicity adjusting agents include for instance dextrose, glycerol, sodium chloride, glycerin and mannitol.

The pharmaceutical formulations may comprise a bulking agent. Bulking agents are particularly useful when the pharmaceutical composition is to be lyophilized before administration. In some embodiments, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbital, glucose and raffinose.

The pharmaceutical formulations may comprise a preservative. Suitable preservatives include for instance antioxidants and antimicrobial agents. However, in preferred embodiments, the pharmaceutical formulation is prepared under sterile conditions and is in a single use container, and thus does not necessitate inclusion of a preservative.

The pharmaceutical formulations of the present disclosure are suitable for parenteral administration. That is the pharmaceutical formulations of the present disclosure are not intended for enteral administration (e.g., not by orally, gastrically, or rectally).

### B. Adjuvants

Pharmaceutically acceptable adjuvants of the present disclosure include for instance, an aluminum salt adjuvant, a squalene-in-water emulsion, a saponin, or combinations thereof. In some embodiments, the adjuvant is an aluminum salt adjuvant selected from the group consisting of amorphous aluminum hydroxyphosphate sulfate, aluminum hydroxide, aluminum phosphate, potassium aluminum sulfate, and combinations thereof. In other embodiments, the adjuvant is a squalene-in-water emulsion such as MF59 or ASO3. In other embodiments, the adjuvant is a saponin, such as Quil A or QS-21, as in ASO1 or ASO2.

### VI. Methods for Production

The present disclosure relates, in some aspects, to methods for preparing hyperactivated dendritic cells, and methods for preparing immunogenic compositions. The immunogenic compositions are suitable for hyperactivation of dendritic cells in vitro, ex vivo, or in vivo.

In one aspect, the present disclosure provides a method for production of hyperactivated dendritic cells (DCs), the method comprising contacting dendritic cells with effective amounts of an isolated lysophosphatidylcholine (LPC) with a single acyl chain, and a PRR agonist for production of hyperactivated dendritic cells, wherein the hyperactivated dendritic cells secrete IL-1beta without undergoing pyroptosis. In some embodiments, the DCs are isolated, while in other embodiments, the DCs are present within a biological sample obtained from a mammalian subject, such as a human patient. In some embodiments, the DCs are monocyte-derived DCs, preferably cDCIs.

In another aspect, the present disclosure provides a method for production of an immunogenic composition, the method comprising combining an antigen with effective amounts of an isolated lysophosphatidylcholine (LPC) with a single acyl chain, and a PRR agonist for production of an immunogenic composition. In some embodiments, the antigen comprises a proteinaceous antigen that is present in or purified from a biological sample obtained from a mammalian subject. In some embodiments, the proteinaceous antigen is a synthetic or recombinant protein. In some preferred embodiments, the antigen is a tumor antigen. In some preferred embodiments, the antigen is a microbial antigen.

In specific embodiments, the present disclosure provide a method for production of an immunogenic composition, the method comprising:
a) depleting leukocytes from a suspension of cells prepared from a tumor to obtain a tumor cell-enriched suspension;
b) lysing cells from the tumor cell-enriched suspension to obtain a tumor cell lysate; and
c) contacting the tumor cell lysate with an isolated lysophosphatidylcholine (LPC) having a single acyl chain and a PRR agonist to obtain the immunogenic composition. In some embodiments, the leukocytes are depleted from the tumor cell-enriched cell suspension by contacting the tumor cell-enriched suspension with an antibody specific to leukocytes. In some embodiments, the leukocytes are depleted by contacting the tumor cell-enriched suspension with an anti-CD45 antibody. In some embodiments, the cells are lysed by a physical disruption-based cell lysis method, such as, but not limited to, mechanical lysis, liquid homogenization, sonication, freeze-thaw, or manual grinding. In some preferred embodiments, the cells are lysed by one or more freeze-thaw cycles.

In some embodiments of the afore-mentioned methods, the acyl chain of the LPC is a C13-C22 acyl chain or a C13-C24 acyl chain. In some embodiments, the acyl chain of the LPC is a C18-C22 acyl chain or a C18-C24 acyl chain. In some preferred embodiments, the acyl chain is fully saturated. In some preferred embodiments, the acyl chain of the LPC is a C22 acyl chain. In some preferred embodiments, the LPC is 1-behenoyl-2-hydroxy-sn-glycero-3-phosphocholine [LPC(22:0)]. In some embodiments, the PRR agonist is a TLR7/8 agonist. In some preferred embodiments, the TLR7/8 agonist is an imidazoquinoline compound, which in particularly preferred embodiments is resiquimod (R848).

### VII. Methods of Use

In some aspects, the present disclosure relates to methods of use of any one of the compositions or formulations described herein, which comprise an LPC compound, a PRR agonist, a dendritic cell, an antigen, an adjuvant, or any combination thereof. The methods of use are suitable for a plurality of uses involving stimulating an immune response. In some embodiments, the methods of use comprise methods of treating cancer. In some embodiments, the methods of use comprise methods of inhibiting abnormal cell proliferation. In some embodiments, the methods of use comprise methods of treating an infectious disease. The methods comprise administering an effective amount of a formulation or a composition described herein to an individual in need thereof to achieve a specific outcome. The individual is a mammalian subject, such as a human patient. In other embodiments, the individual is a non-human patient. In some embodiments, the individual is a canine patient. That is in some embodiments, the methods of use involve clinical uses, while in other embodiments the methods of use involve pre-clinical and/or veterinary uses. For preclinical uses, the mammalian subject may be a non-human primate (e.g., monkey or ape) or a rodent (e.g., mouse or rat). For veterinary uses the mammalian subject may be a farm animal (e.g., cow), a sport animal (e.g., horse), or a pet (e.g., companion animal such as a dog or cat).

### A. Stimulation of an Immune Response

In brief, the present disclosure provides methods of stimulating an immune response in an individual, comprising administering to the individual a composition or formulation described herein in an amount sufficient to stimulate an immune response in the individual. “Stimulating” an immune response (used interchangeably with “eliciting” an immune response), means increasing the immune response, which can arise from eliciting a de novo immune response (e.g., as a consequence of an initial vaccination regimen) or enhancing an existing immune response (e.g., as a consequence of a booster vaccination regimen). In some embodiments, stimulating an immune response comprises one or more of the group consisting of: stimulating cytokine production; stimulating B lymphocyte proliferation; stimulating interferon pathway-associated gene expression; stimulating chemoattractant-associated gene expression; and stimulating dendritic cell DC maturation. Methods for measuring stimulation of an immune response are known in the art.

For instance, the present disclosure provides methods of inducing an antigen-specific immune response in an individual by administering to the individual a composition or formulation described herein in an amount sufficient to induce an antigen-specific immune response in the individual. In preferred embodiments, the composition or formulation comprises the antigen. In some embodiments, the composition or formulation is administered to a tissue of the individual comprising the antigen. The immune response may comprise one or both of an antigen-specific antibody response and an antigen-specific cytotoxic T lymphocyte (CTL) response. “Inducing” an antigen-specific antibody response means increasing titer of the antigen-specific antibodies above a threshold level such as a pre-administration baseline titer or a seroprotective level. “Inducing” an antigen-specific CTL response means increasing frequency of antigen-specific CTL found in peripheral blood above a pre-administration baseline frequency.

Analysis (both qualitative and quantitative) of the immune response can be by any method known in the art, including, but not limited to, measuring antigen-specific antibody production (including measuring specific antibody subclasses), activation of specific populations of lymphocytes such as B cells and helper T cells, production of cytokines such as IFN-alpha, IFN-gamma, IL-6, IL-12 and/or release of histamine. Methods for measuring antigen-specific antibody responses include enzyme-linked immunosorbent assay (ELISA). Activation of specific populations of lymphocytes can be measured by proliferation assays, and with fluorescence-activated cell sorting (FACS). Production of cytokines can also be measured by ELISA. In some embodiments, methods of stimulating an immune response comprise stimulation of interleukin-1beta (IL-1β) secretion, interferon-gamma (IFN-γ) secretion, and/or tumor necrosis factor-alpha (TNF-α) secretion by monocyte-derived dendritic cells or peripheral blood mononuclear cells. In some embodiments, methods of stimulating an immune response comprise stimulation of secretion of one or more of IFN-γ, IL-17a, IL-17f, and IL-22 by memory CD4+ T cells. In some embodiments, methods of stimulating an immune response comprise increasing Th1 differentation of naïve CD4+ T cells. In some preferred embodiments, at least 50%, 55%, 60%, 65%, 70% or 75% of the cells contacted with a composition of the present disclosure remain viable at 40-56 hours (or about 48 hours) post-contact.

In some embodiments, the methods are suitable for stimulating an anti-tumor immune response. In other embodiments, the methods are suitable for stimulating an anti-microbe immune response. In some embodiments, the anti-microbe response is an anti-bacterial immune response. In some embodiments, the anti-microbe response is an anti-fungal immune response. In some embodiments, the anti-microbe response is, an anti-viral immune response. In some embodiments, the anti-microbe response is an anti-protozoan immune response.

### B. Treating or Preventing Disease

The present disclosure further provides methods of treating or preventing a disease in an individual, comprising administering to the individual a composition or formulation described herein in an amount sufficient to treat or prevent a disease in the individual. In some embodiments, the disease is cancer. In some embodiments, the disease is abnormal cell proliferation. In other embodiments, the disease is an infectious disease.

In one aspect, the methods may comprise administering a composition comprising an LPC compound, a PRR agonist, an antigen, an adjuvant, or any combination thereof, to a subject in need thereof. In another aspect, the methods involve adoptive cell therapy, and comprise administering a composition comprising a dendritic cell, such as a hyperactivated dendritic cell, and an LPC compound, a PRR agonist, an antigen, an adjuvant, or any combination thereof, to a subject in need thereof.

In some embodiments, the methods involve treating cancer in an individual or otherwise treating a mammalian subject with cancer. In some embodiments, the methods comprise: a) preparing an immunogenic composition comprising a tumor cell lysate, an isolated lysophosphatidylcholine (LPC) having a single acyl chain, and a toll-like receptor 7/8 (TLR7/8) agonist, wherein the tumor cell lysate is or has been prepared from a sample of a tumor obtained from the subject with cancer, and the acyl chain is a C13-C22 acyl chain or a C13-C24 acyl chain; and b) administering to the subject an effective amount of the immunogenic composition. In some embodiments, the cancer is a hematologic cancer, such as a lymphoma, a leukemia, or a myeloma. In other embodiments, the cancer is a non-hematologic cancer, such as a sarcoma, a carcinoma, or a melanoma. In some embodiments, the cancer is malignant.

In some embodiments, the methods involve inhibiting abnormal cell proliferation in an individual. “Abnormal cell proliferation” refers to proliferation of a benign tumor or a malignant tumor. The malignant tumor may be a metastatic tumor.

In some embodiments, the methods involve treating or preventing an infectious disease in an individual. In some embodiments, the infectious disease is caused by a viral infection. In other embodiments, the infectious disease is caused by a bacterial infection. In further embodiments, the infectious disease is caused by a fungal infection. In still further embodiments, the infectious disease is caused by a protozoal infection. Of particular importance are infectious diseases caused by zoonotic pathogens that infect humans as well as other animals such as mammals or birds. In some embodiments, the zoonotic pathogen is transmitted to humans via an intermediate species (vector).

### ENUMERATED EMBODIMENTS

1. A composition comprising an isolated lysophosphatidylcholine (LPC) with a single acyl chain, and a TLR7/8 agonist, wherein the acyl chain is a C13-C22 acyl chain or a C13-C24 acyl chain.

2. The composition of embodiment 1, wherein the acyl chain is a C18-C22 acyl chain or a C21-C24 acyl chain.

3. The composition of embodiment 1 or embodiment 2, further comprising an antigen.

4. The composition of any one of embodiments 1-3, further comprising dendritic cells.

5. A composition comprising an isolated lysophosphatidylcholine (LPC) with a single acyl chain, and an antigen, wherein the acyl chain is a C21-C24 acyl chain.

6. The composition of embodiment 5, further comprising dendritic cells.

7. The composition of embodiment 5 or embodiment 6, further comprising a TLR7/8 agonist.

8. A composition comprising an isolated lysophosphatidylcholine (LPC) with a single acyl chain, and dendritic cells, wherein the acyl chain is a C21-C24 acyl chain.

9. The composition of embodiment 8, further comprising a TLR7/8 agonist.

10. The composition of embodiment 8 or embodiment 9, further comprising an antigen.

11. A composition of any one of embodiments 1-10, wherein the acyl chain is a C22 acyl chain.

12. The composition of any one of embodiments 1-11, wherein the acyl chain is fully saturated.

13. The composition of any one of embodiments 1-12, wherein the LPC comprises 1-behenoyl-2-hydroxy-sn-glycero-3-phosphocholine [LPC(22:0)].

14. The composition of any one of embodiments 1-13, wherein the TLR7/8 agonist is a small molecule with a molecule weight of 900 daltons or less.

15. The composition of embodiment 14, wherein the TLR7/8 agonist comprises an imidazoquinoline compound.

16. The composition of embodiment 15, wherein the TLR7/8 agonist comprises resiquimod (R848).

17. The composition of embodiment 14 or embodiment 15, wherein the TLR7/8 agonist does not inhibit NLR family pyrin domain containing 3 (NLRP3).

18. The composition of embodiment 13, wherein the LPC comprises LPC(22:0), and the TLR7/8 agonist comprises resiquimod (R848).

19. The composition of any one of embodiments 1-18, wherein the antigen is present in a biological sample obtained from an individual.

20. The composition of embodiment 19, wherein the biological sample comprises biopsy tissue.

21. The composition of embodiment 19, wherein the biological sample comprises cells.

22. The composition of embodiment 19, wherein the biological sample does not comprise cells.

23. The composition of embodiment 19, wherein the biological sample comprises pus from an abscess.

24. The composition of any one of embodiments 1-23, wherein the antigen comprises a proteinaceous antigen.

25. The composition of embodiment 24, wherein the antigen comprises a tumor antigen.

26. The composition of embodiment 25, wherein the tumor antigen comprises a synthetic or recombinant neoantigen.

27. The composition of embodiment 26, wherein the tumor antigen comprises a tumor cell lysate.

28. The composition of embodiment 24, wherein the antigen comprises a microbial antigen and the microbial antigen comprises one or more of a viral antigen, a bacterial antigen, a protozoan antigen, and a fungal antigen.

29. The composition of embodiment 28, wherein the microbial antigen comprises a purified or recombinant surface protein.

30. The composition of embodiment 28, wherein the microbial antigen comprises an inactivated, whole virus.

31. The composition of any one of embodiments 1-30, wherein the composition does not comprise liposomes.

32. The composition of any one of embodiments 1-31, wherein the composition does not comprise LPS or MPLA.

33. The composition of any one of embodiments 1-32, wherein the composition does not comprise oxPAPC or a species of oxPAPC.

34. The composition of embodiment 33, wherein the composition does not comprise HOdiA-PC, KOdiA-PC, HOOA-PC, KOOA-PC, and/or PGPC.

35. The composition of any one of embodiments 1-34, further comprising an adjuvant, wherein the adjuvant comprises an aluminum salt adjuvant, a squalene-in-water emulsion, a saponin, or combinations thereof.

36. A pharmaceutical formulation comprising the composition of any one of embodiments 1-35 and a pharmaceutically acceptable excipient.

37. A method for production of hyperactivated dendritic cells, the method comprising contacting the dendritic cells with a composition comprising effective amounts of an isolated lysophosphatidylcholine (LPC) with a single C13-C22 acyl chain or a single C13-C24 acyl chain, and a TLR7/8 agonist for production of hyperactivated dendritic cells, wherein the hyperactivated dendritic cells secrete IL-1beta without undergoing pyroptosis.

38. The method of embodiment 37, wherein the dendritic cells are contacted ex vivo with the composition of any one of embodiments 1-35 or the formulation of embodiment 36.

39. The method of embodiment 37, wherein the dendritic cells are contacted in vivo with the formulation of embodiment 36.

40. A pharmaceutical formulation comprising at least 10³, 10⁴, 10⁵ or 10⁶ of the hyperactivated dendritic cells produced by the method of embodiment 38, and a pharmaceutically acceptable excipient.

41. A method of stimulating an immune response against an antigen, comprising administering an effective amount of the formulation of embodiment 36 to an individual in need thereof to stimulate the immune response against the antigen.

42. A method of treating cancer, comprising administering an effective amount of the formulation of embodiment 36 to an individual in need thereof to treat the cancer.

43. A method of inhibiting abnormal cell proliferation, comprising administering an effective amount of the formulation of embodiment 36 to an individual in need thereof to inhibit abnormal cell proliferation.

44. A method of treating an infectious disease, comprising administering an effective amount of the formulation of embodiment 36 to an individual in need thereof to treat the infectious disease.

45. Use of the formulation of embodiment 36 for inducing an immune response against the antigen in an individual in need thereof.

46. Use of the formulation of embodiment 36 for inducing an anti-tumor immune response in an individual in need thereof, wherein the individual is or was tumor-bearing.

47. Use of the formulation of embodiment 36 for inducing an anti-microbe immune response in an individual in need thereof, wherein the individual is infected with the microbe or has not been exposed to the microbe.

48. The composition, formulation, method or use of any one of embodiments 19-47, wherein the individual is a mammalian subject.

49. The composition, formulation, method or use of any one of embodiments 19-47, wherein the individual is a human subject.

50. A method of preparing an immunogenic composition, the method comprising:
a) depleting leukocytes from a suspension of cells prepared from a tumor to obtain a tumor cell-enriched suspension;
b) lysing cells from the tumor cell-enriched suspension to obtain a tumor cell lysate; and
c) contacting the tumor cell lysate with an isolated lysophosphatidylcholine (LPC) having a single acyl chain and a toll-like receptor 7/8 (TLR7/8) agonist to obtain the immunogenic composition, wherein the acyl chain is a C13-C22 acyl chain or a C13-C24 acyl chain.

51. The method of embodiment 50, wherein the leukocytes are depleted in step a) by negative selection using an anti-CD45 antibody.

52. The method of embodiment 50 or embodiment 51, wherein the cells are lysed in step b) by one or more freeze-thaw cycles.

53. The method of any one of embodiments 50-52, wherein the acyl chain is a fully saturated C18-C22 acyl chain or a fully saturated C18-C24 acyl chain.

54. The method of embodiment 53, wherein the LPC comprises 1-behenoyl-2-hydroxy-sn-glycero-3-phosphocholine [LPC(22:0)].

55. The method of any one of embodiments 50-54, wherein the TLR7/8 agonist is a small molecule with a molecule weight of 900 daltons or less.

56. The method of embodiment 55, wherein the TLR7/8 agonist comprises an imidazoquinoline compound.

57. The method of embodiments 56, wherein the TLR7/8 agonist comprises resiquimod (R848).

58. The method of embodiment 55 or embodiment 56, wherein the TLR7/8 agonist does not inhibit NLR family pyrin domain containing 3 (NLRP3).

59. The method of embodiment 54, wherein the LPC comprises LPC(22:0), and the TLR7/8 agonist comprises resiquimod (R848).

60. The method of any one of embodiments 50-59, further comprising before step a) obtaining a sample from the tumor from a mammalian subject with cancer and preparing the suspension of cells from the sample.

61. An immunogenic composition prepared by the method of any one of embodiments 50-60.

62. A method of eliciting an anti-cancer immune response, the method comprising: administering to a mammalian subject with cancer an effective amount of the immunogenic composition of embodiment 61.

63. The method of embodiment 62, wherein the anti-cancer immune response comprises cellular immune response.

64. The method of embodiment 63, wherein the anti-cancer immune response comprises cancer antigen-induced IL-1beta secretion and/or activation of CD8+T lymphocytes.

65. The method of any one of embodiments 62-64, wherein the cancer is a non-hematologic cancer.

66. The method of embodiment 65, wherein the non-hematologic cancer is a carcinoma, a sarcoma, or a melanoma.

67. The method of any one of embodiments 62-64, wherein the cancer is a lymphoma.

68. A method of treating cancer, the method comprising:
a) preparing an immunogenic composition comprising a tumor cell lysate, an isolated lysophosphatidylcholine (LPC) having a single acyl chain, and a toll-like receptor 7/8 (TLR7/8) agonist, wherein the tumor cell lysate is or has been prepared from a sample of a tumor obtained from the mammalian subject with cancer, and the acyl chain is a C13-C22 acyl chain or a C13-C24 acyl chain; and
b) administering to the subject an effective amount of the immunogenic composition.

69. The method of any one of embodiments 62-68, wherein the acyl chain is a fully saturated C18-C22 acyl chain or a fully saturated C18-C24 acyl chain.

70. The method of embodiment 68, wherein the LPC comprises 1-behenoyl-2-hydroxy-sn-glycero-3-phosphocholine [LPC(22:0)].

71. The method of any one of embodiments 62-70, wherein the TLR7/8 agonist is a small molecule with a molecule weight of 900 daltons or less.

72. The method of embodiment 71, wherein the TLR7/8 agonist comprises an imidazoquinoline compound.

73. The method of embodiment 72, wherein the TLR7/8 agonist comprises resiquimod (R848).

74. The method of embodiment 70, wherein the LPC comprises 22:0 LPC, and the TLR7/8 agonist comprises resiquimod (R848).

75. The method of any one of claims 68-74, further comprising administering to the subject an effective amount of an additional therapeutic agent.

76. The method of embodiment 75, wherein the additional therapeutic agent comprises one or more of the group consisting of an immune checkpoint inhibitor, an antineoplastic agent, and radiation therapy.

77. A composition comprising an isolated lysophosphatidylcholine (LPC) with a single acyl chain, and a pathogen recognition receptor (PRR) agonist, wherein the acyl chain is a C13-C22 acyl chain or a C13-C24 acyl chain.

78. The composition of embodiment 77, wherein the PRR agonist is an agonist of a toll-like receptor (TLR), a NOD-like receptor (NLR), a RIG-I-like receptor (RLR), or a C-type lectin receptor (CLR).

79. The composition of embodiment 77, wherein the PRR agonist is an agonist of a cytosolic DNA sensor (CDS) or a stimulator of IFN genes (STING).

80. The composition of embodiment 77, wherein the PRR agonist comprises one or more of R848, TL8-506, LPS, Pam2CSK4, and ODN 2336.

81. The composition of any one of embodiments 77-80, further comprising an antigen.

82. The composition of any one of embodiments 77-81, further comprising dendritic cells.

83. A pharmaceutical formulation comprising the composition of any one of embodiments 77-82 and a pharmaceutically acceptable excipient.

84. A pharmaceutical formulation comprising an isolated lysophosphatidylcholine (LPC) with a single acyl chain, and a pharmaceutically acceptable excipient, wherein the acyl chain is a C21-C24 acyl chain.

85. The pharmaceutical formulation of embodiment 83 or embodiment 84, wherein the acyl chain is a fully saturated C22 acyl chain.

86. The pharmaceutical formulation of embodiment 85, wherein the LPC comprises 1-behenoyl-2-hydroxy-sn-glycero-3-phosphocholine [LPC(22:0)].

87. A composition for hyperactivation of human dendritic cells, comprising an isolated lysophosphatidylcholine (LPC) with a single acyl chain, and a pathogen recognition receptor (PRR) agonist, wherein the acyl chain is C22 acyl chain, and wherein the composition is effective for achieving a higher level of dendritic cell hyperactivation than a comparator composition comprising PGPC in place of the LPC.

88. The composition of embodiment 87, wherein the higher level of dendritic cell hyperactivation comprises induction of IL-1beta secretion from the human dendritic cells in vitro at a level that is at least 2, 3 or 4 fold higher when contacted with the composition comprising the LPC and the PRR agonist than when contacted with the comparator composition comprising the PGPC and the PRR agonist, wherein the PRR agonist is LPS.

89. The composition of embodiment 88, wherein the concentration of the LPC and the concentration of the PGPC are the same concentration in a range of from about 10 μM to about 80 μM, and the LPS is present at a concentration of 1 μg/ml in both the composition and the comparator composition.

90. The composition of embodiment 88, wherein the higher level of dendritic cell hyperactivation comprises a lipid activity index for IL-1beta secretion from the human dendritic cells for the composition comprising the LPC and the PRR agonist that is at least 4, 5 or 6 fold higher in activity units than that of the comparator composition comprising the LPC and the PRR agonist.

91. The composition, formulation, method or use of any one of embodiments 19-47, wherein the individual is a canine subject.

92. The composition, formulation, method or use of any one of embodiments 60-90, wherein the mammalian subject is a human patient.

93. The composition, formulation, method or use of any one of embodiments 60-90, wherein the mammalian subject is a non-human patient.

94. The composition, formulation, method or use of any one of embodiments 60-90, wherein the mammalian subject is a canine patient.

95. The composition, formulation, method or use of any one of embodiment 1-90 or 92, wherein the dendritic cells are human dendritic cells.

96. The composition, formulation, method or use of any one of embodiment 1-91 or 94, wherein the dendritic cells are canine dendritic cells.

97. The composition, method or use of embodiment 95 or embodiment 96, wherein the dendritic cells are present in a composition comprising peripheral blood mononuclear cells (PBMCs).

98. The composition, method or use of any one of embodiments 37-49 or embodiment 91, wherein the hyperactivated dendritic cells secrete one or both of IFNγ and TNFα.

99. The composition, formulation, method or use of any one of embodiments 1-98, comprising a surfactant.

100. The composition, formulation, method or use of embodiment 99, wherein the surfactant comprises a non-ionic surfactant.

101. The composition, formulation, method or use of embodiment 100, wherein the non-ionic surfactant comprises an ethylene oxide-propylene oxide copolymer.

102. The composition, formulation, method or use of embodiment 100, wherein the non-ionic surfactant comprises one or more of Poloxamer 407, Poloxamer 188, and P123.

103. The composition, formulation, method or use of embodiment 100, wherein the non-ionic surfactant comprises Poloxamer 407.

104. The composition, formulation, method or use of any one of embodiments 100-103, wherein i) the LPC is dissolved in an alcohol to form an LPC alcohol solution; ii) the LPC alcohol solution is mixed with the non-ionic surfactant to form a mixture; and iii) the alcohol is evaporated from the mixture to form particles comprising the LPC and the non-ionic surfactant.

105. The composition, formulation, method or use of any one of embodiments 100-104, wherein the non-ionic surfactant is present in an amount of about 2.5% to 25% (w/w), optionally about 5% to 20% (w/w), optionally about 15% (w/w).

106. The composition, formulation, method or use of any one of embodiments 100-105, wherein the LPC and non-ionic surfactant are present in particles with a diameter of about 1000 to 2000 nanometers, optionally with a diameter of about 1500 nanometers.

### EXAMPLES

Abbreviations: BM (bone marrow); BMDC (bone marrow-derived dendritic cell); CDS (cytosolic DNA sensor); CLR (C-type lectin receptor); DAMP (damage-associated molecular pattern); DC (dendritic cell); dLN (draining lymph node); HOdiA-PC (1-Palmitoyl-2-(5-hydroxy-8-oxo-6-octenedioyl)-sn-glycero-3-phosphatidylcholine); HOOA-PC (1-palmitoyl-2-(5-hydroxy-8-oxooct-6-enoyl)-sn-glycero-3-phosphocholine); IFNγ (interferon-gamma); IL-1b/IL1-beta/IL-1β (Interleukin-1beta); KOdiA-PC (1-(Palmitoyl)-2-(5-keto-6-octene-dioyl) phosphatidylcholine); KOOA-PC (1-palmitoyl-(5-keto-8-oxo-6-octenoyl)-sn-glycero-3-phosphocholine); KP407 (poloxamer 407); LPC/Lyso PC (lysophosphatidylcholine); Lyso PC(22:0) (1-behenoyl-2-hydroxy-sn-glycero-3-phosphocholine); LPS (lipopolysaccharide); MFI (mean fluorescence intensity); moDC (monocyte-derived dendritic cell); MPLA (monophosphoryl lipid A); NLR (NOD-like receptor); oxPAPC (oxidized 1-palmitoyl-2-arachidonyl-sn-glycero-3-phosphorylcholine); PAMP (pathogen-associated molecular pattern); PBMCs (peripheral blood mononuclear cells); PGPC (1-palmitoyl-2-glutaryl-sn-glycero-3-phosphocholine); POVPC (1-palmitoyl-2-(5′-oxo-valeroyl)-sn-glycero-3-phosphocholine); PRR (pathogen recognition receptor); RLR (RIG-I-like receptor); R848 (resiquimod); STING (stimulator of IFN genes); TNFα (tumor necrosis factor-alpha); TLR (toll-like receptor); and WTL (whole tumor lysate).

Although the present disclosure has been described in some detail by way of illustration and example for purposes of clarity and understanding, it will be apparent to those skilled in the art that certain changes and modifications may be practiced. Therefore, the following examples should not be construed as limiting the scope of the present disclosure, which is delineated by the appended claims.

### Example 1: Identification of DAMPs and PAMPs for Hyperactivation of Dendritic Cells

This example describes the identification of lipid DAMPs and a small molecule PAMP that in combination are able to hyperactivate human dendritic cells.

### Materials and Methods

Differentiation of human monocyte-derived dendritic cells (moDCs). Human monocytes were isolated from Leukopaks (purchased from Miltenyi) using the StraightFrom Leukopak CD14 microbead kit (Miltenyi), following manufacturer's instructions. Monocytes were then aliquoted and frozen in fetal bovine serum containing 10% dimethyl sulfoxide. For studies of hyperactivating lipids using moDC cultures, monocytes were thawed and cultured in RPMI medium containing 10% FBS, 50 units/mL penicillin, 50 mg/mL streptomycin, 2 mM L-glutamine, 1 mM sodium pyruvate, and 50 mM beta-mercaptoethanol (R10 media). Human monocytes were differentiated using recombinant human GM-CSF (50 ng/mL) and IL-4 (25 ng/mL), added to R10 media. Cells were cultured for 6 days with GM-CSF and IL-4, with an additional cell feeding of R10 containing GM-CSF and IL-4 on day 3.

Danger-Associated Molecular Pattern (DAMP) Screen. After 6 days of differentiation into moDC, cells were collected and counted. Cells were plated at 100,000 cells/well in R10 media in 96-well flat bottom tissue culture-treated plates. LPS, serotype O55:B5 (Enzo Life Sciences) was added to a final concentration of 1 μg/mL in each well. After the addition of LPS, lipids were prepared and added at a final concentration of 82.5 μM or 41.3 μM. Cells were incubated at 37° C., 5% CO₂ for two days. Cell cultures were then used for endpoint analyses.

Lipid Titration. After 6 days of differentiation into moDC, cells were collected and counted. Cells were replated at 100,000 cells/well in R10 media in 96-well flat bottom tissue culture-treated plates. LPS, serotype 055:B5 (Enzo Life Sciences) was added to a final concentration of 1 μg/mL in each well. After the addition of LPS, lipids were prepared. A working stock of the highest tested lipid concentration (82.5 μM) was made in R10 and then sequential two-fold dilutions were made to the final tested concentration, 1.3 μM. After two days of culture, cell cultures were used for endpoint analyses.

Pathogen-Associated Molecular Pattern (PAMP) Screen. Innate immune signaling pathway agonists were purchased from Invivogen. Lyophilized stocks were reconstituted and stored according to manufacturer's instructions and at recommended concentrations. After 6 days of differentiation into moDC, cells were collected and counted. Cells were replated at 100,000 cells/well in R10 media in 96-well flat bottom tissue culture-treated plates. Innate immune signaling agonists were diluted in R10 media to the manufacturer's recommended working concentration and then added onto cells. 22:0 Lyso PC was then reconstituted from a lyophilized stock in R10 and then further diluted. The lipid was added onto cells at a final concentration of 20 μM. Cells were incubated for 2 days, then cultures were collected for endpoint analyses.

Endpoint Analyses. After culturing moDCs with PAMPs and DAMPs for two days, supernatant and cell samples were collected for analysis. Cells in culture were pelleted by centrifugation at 400×g for 5 min. Half of the media volume in the wells was collected for cytokine quantification by Enzyme-Linked Immunosorbent Assay (ELISA), while the remaining media and cells were used to quantify cell viability by assessing metabolic activity.

Quantification of IL-1beta Secretion. IL-1beta secretion was assessed using the ELISA MAX Deluxe Set Human IL-1beta kit (Biolegend). ELISAs were performed according to manufacturer's instructions with the following modifications: i) total sample volume for incubation was reduced from 100 mL to 50 mL (25 mL/well Assay Buffer D with 25 mL/well samples or standards; ii) the top standard was prepared at 500 μg/mL, with two-fold dilutions to 7.8 μg/mL; and iii) sample incubation was completed overnight at 4 C on an orbital shaker. Absorbance was measured at 450 nm, with a 570 nm correction, using a Spectramax iD3 plate reader (Molecular Devices). To determine IL-1beta concentration in supernatants, sample IL-1beta concentrations were interpolated using a standard curve via 4PL analysis on GraphPad Prism 9 (GraphPad Software). The interpolated results of samples were then adjusted for any dilutions made to the supernatant.

Quantification of Cell Viability. Cell viability was assessed by quantifying the presence of ATP as an indicator of metabolically active cells using the CellTiter-Glo Luminescent Cell Viability Assay (Promega). Metabolic activity was assessed following manufacturer's instructions. The CellTiter-Glo reagent was mixed with the cell pellets and remaining supernatant, and transferred to a white, opaque 96-well plate. Luminescence was measured on all wavelengths on a Spectramax iD3 plate reader (Molecular Devices) using an integration time of 500 ms. Percent viability was calculated relative to the control condition of moDCs treated with LPS.

Statistical Analyses. For each data set, multiple donors were tested, with the number per test indicated in the tables corresponding to each study. For each tested condition, cells from each donor were plated to be tested in triplicate. Each donor triplicate was averaged, and the average was included as one donor measurement when quantifying IL-1beta or measuring viability. On graphs, each data point represents the mean value for a donor. To test for differences in test conditions, tests results were compared to control conditions indicated in each table. P-values were calculated using a repeated measures one-way ANOVA, with corrections for multiple comparisons using a Dunnett's test.

### Results—Lipids of a Defined Structure Have Hyperstimulatory Ability

Typically, dendritic cell (DC) maturation by vaccine adjuvants such as Toll-like receptor agonists (TLRs) does not lead to IL-1β secretion. During inflammasome activation, IL-1β secretion does occur, but may be accompanied by a lytic programmed cell death process termed pyroptosis (Evavold et al., J Mol Biol., 430(2):217-237, 2019). However, when DCs are matured using the PAMP LPS and the DAMP PGPC, they are capable of producing and secreting IL-1β without pyroptosing (see, FIG. 1), characterizing these viable DCs as hyperactive DCs (Zhivaki et al., Cell Rep., 33(7):108381, 2020). In mouse models, hyperactivated DCs have been observed to have an improved ability to induce an immune response compared to cells activated using LPS alone (Zhivaki et al., Cell Rep., 33(7):108381, 2020).

To identify additional lipids that may hyperactivate DCs (e.g., hyperactivating lipids), a library of phosphotidylcholine (PC) lipids was selected for screening. The library of PC lipids included mixed acyl PC lipids, lyso PC lipids, and oxidized PC lipids. While all three types of PC lipids are structurally similar to PGPC due to the presence of a phosphocholine head group, each type varies in the number and length of acyl chains, degree of fatty acid saturation, and state of oxidation. When designing a lipid screening strategy, the importance of these characteristics was unclear because the use of these lipids to hyperactivate DCs, let alone human DCs had not been reported.

In order to identify lipid species most relevant for clinical use, the PC lipids were screened using human DCs. MoDC are activated by LPS, and will produce the precursor to IL-1β (e.g., immature IL-1β), but will not secrete IL-1β in response to LPS alone. Exposure to PGPC in combination with LPS causes moDC to secrete the cleaved, active form of IL-1β, while moDC remain viable. Although secreted IL-1β may be detected one day after DC hyperactivation in cell culture supernatants, cell viability was evaluated two days post-hyperactivation to ensure enduring viability after IL-1β secretion. MoDC viability is essential, as cells that have died due to pyroptosis or toxicity of lipids are not able to interact with other immune cells, and therefore are incapable of stimulating an adaptive T cell response.

For each data set, human monocytes from multiple donors were tested. In brief, human monocyte samples were differentiated into moDC using GM-CSF and IL-4. Cells were plated and then activated with LPS. Lipids in the library were then added into cell cultures at 82.5 μM, and cells were hyperactivated for two days. Many lipid species induced the production of IL-1beta, predominantly in the Lyso PC lipid group (Table 1-1). Lipids had varying effects on cell viability as well, with lipids inducing IL-1beta secretion sometimes lowering cell viability (Table 1-1).

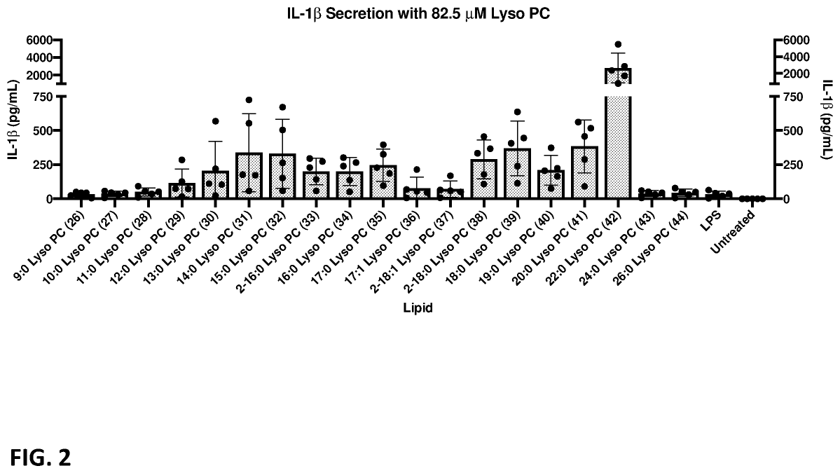

To discern a relationship between lipid structure and hyperactivating capacity, the largest group of lipids that stimulate IL-1beta secretion (Lyso PC) was further analyzed. The Lyso PC lipids tested all have a single acyl chain, spanning from 6 to 26 carbons in length. For all donors tested, IL-1beta secretion was detectable above the LPS-treated control condition when lipids had single acyl chains longer than 12 carbons in length (FIG. 2), indicating that PC lipids with a single acyl chain 12-22 carbons in length can stimulate IL-1beta secretion. Surprisingly, unsaturated acyl chains 12-22 carbons in length did not induce IL-1beta secretion, as seen with tests including lipids 36 and 37 (Table 1-1 and FIG. 2). In contrast, lipids with equally long saturated acyl chains (lipid tests 35 and 38) were able to induce IL-1beta secretion (Table 1-1 and FIG. 2). The double bond found in lipid species 36 and 37 renders these lipids inactive, possibly because of the changes to their structures.

Further, the positioning of the single acyl chain does not seem to matter, indicated by lipid tests 38 and 39 (Table 1-1 and FIG. 2). Instead, the number of acyl chains seems to play a larger role. Lipids within the Mixed Acyl PC subset have two acyl chains of varying length. Despite having acyl chains within the 12-22 carbon length range, these lipids were largely inactive.

TABLE 1-1
IL-1β Secretion and Relative Viability (82.5 μM Lipid Treatment)\hat{ }

| | | | | | | Relative | | No. Donors |
|---|---|---|---|---|---|---|---|---|
| | | | | IL-1β | p- | Viability | p- | (Replicates) |
| Test | Lipid | Class | PAMP | secretion | value | (%) ± SD | value | Tested |
| 1 | 15:0-18:1 | Mixed | LPS | 45.9 ± | ns* | 90.5 ± | 0.0331 | 5 (3) |
| | PC | Acyl | | 15.3 | | 3.4 | | |
| 2 | 14:0-16:0 | Mixed | LPS | 53.1 ± | ns | 90.8 ± | ns* | 5 (3) |
| | PC | Acyl | | 27.7 | | 5.9 | | |
| 3 | 14:0-18:0 | Mixed | LPS | 34.6 ± | ns | 90.5 ± | ns | 5 (3) |
| | PC | Acyl | | 17.7 | | 5.5 | | |
| 4 | 16:0-14:0 | Mixed | LPS | 62.5 ± | ns | 87.4 ± | ns | 5 (3) |
| | PC | Acyl | | 44.2 | | 7.9 | | |
| 5 | 16:0-18:0 | Mixed | LPS | 42.7 ± | ns | 88.8 ± | ns | 5 (3) |
| | PC | Acyl | | 20.8 | | 5.6 | | |
| 6 | 16:0-18:1 | Mixed | LPS | 32.9 ± | ns | 92.9 ± | ns | 5 (3) |
| | PC (POPC) | Acyl | | 9.1 | | 4.0 | | |
| 7 | 16:0-18:2 | Mixed | LPS | 35.1 ± | ns | 93.5 ± | ns | 5 (3) |
| | PC | Acyl | | 17.2 | | 4.2 | | |
| 10 | 18:0-14:0 | Mixed | LPS | 42.2 ± | ns | 86.1 ± | ns | 5 (3) |
| | PC | Acyl | | 22.8 | | 5.1 | | |
| 11 | 18:0-16:0 | Mixed | LPS | 38.9 ± | ns | 85.3 ± | ns | 5 (3) |
| | PC | Acyl | | 27.3 | | 10.3 | | |
| 12 | 18:0-18:1 | Mixed | LPS | 27.2 ± | ns | 95.6 ± | ns | 5 (3) |
| | PC | Acyl | | 13.9 | | 3.7 | | |
| 13 | 18:0-18:2 | Mixed | LPS | 27.3 ± | ns | 101.0 ± | ns | 5 (3) |
| | PC | Acyl | | 13.4 | | 7.3 | | |
| 14 | 18:0-20:4 | Mixed | LPS | 38.5 ± | ns | 87.1 ± | ns | 5 (3) |
| | PC | Acyl | | 20.9 | | 5.2 | | |
| 16 | 18:1-14:0 | Mixed | LPS | 35.9 ± | ns | 102.5 ± | ns | 5 (3) |
| | PC | Acyl | | 16.9 | | 3.4 | | |
| 17 | 18:1-16:0 | Mixed | LPS | 34.2 ± | ns | 95.3 ± | ns | 5 (3) |
| | PC | Acyl | | 17.1 | | 2.3 | | |
| 18 | 18:1-18:0 | Mixed | LPS | 30.1 ± | ns | 95.0 ± | 0.0443 | 5 (3) |
| | PC | Acyl | | 15.7 | | 2.0 | | |
| 19 | 18:1(n10)- | Mixed | LPS | 26.8 ± | ns | 97.8 ± | ns | 5 (3) |
| | 16:0 PC | Acyl | | 16.2 | | 1.6 | | |
| 21 | 16:0-(12- | Mixed | LPS | 46.5 ± | ns | 96.9 ± | ns | 5 (3) |
| | PAHSA) PC | Acyl | | 25.0 | | 3.8 | | |
| 22 | 18:1 Δ⁹ | Lyso | LPS | 44.8 ± | ns | 91.5 ± | ns | 5 (3) |
| | LYSO PC | | | 38.5 | | 3.2 | | |
| 23 | 06:0 LYSO | Lyso | LPS | 44.4 ± | ns | 92.8 ± | 0.0329 | 5 (3) |
| | PC | | | 18.9 | | 2.0 | | |
| 24 | 07:0 LYSO | Lyso | LPS | 35.9 ± | ns | 93.9 ± | ns | 5 (3) |
| | PC | | | 19.9 | | 3.3 | | |
| 25 | 08:0 LYSO | Lyso | LPS | 34.3 ± | ns | 95.3 ± | ns | 5 (3) |
| | PC | | | 18.6 | | 3.2 | | |
| 26 | 09:0 LYSO | Lyso | LPS | 33.7 ± | ns | 95.4 ± | ns | 5 (3) |
| | PC | | | 18.5 | | 3.7 | | |
| 27 | 10:0 LYSO | Lyso | LPS | 37.8 ± | ns | 95.2 ± | ns | 5 (3) |
| | PC | | | 19.2 | | 4.2 | | |
| 28 | 11:0 LYSO | Lyso | LPS | 49.6 ± | ns | 92.8 ± | ns | 5 (3) |
| | PC | | | 29.7 | | 3.4 | | |
| 29 | 12:0 LYSO | Lyso | LPS | 115.3 ± | ns | 91.1 ± | ns | 5 (3) |
| | PC | | | 103.0 | | 5.5 | | |
| 30 | 13:0 LYSO | Lyso | LPS | 206.2 ± | ns | 93.2 ± | ns | 5 (3) |
| | PC | | | 214.7 | | 5.5 | | |
| 31 | 14:0 LYSO | Lyso | LPS | 337.8 ± | 0.0014 | 79.2 ± | 0.0488 | 5 (3) |
| | PC | | | 286.2 | | 7.0 | | |
| 32 | 15:0 LYSO | Lyso | LPS | 330.9 ± | 0.0020 | 65.4 ± | 0.0087 | 5 (3) |
| | PC | | | 253.0 | | 6.4 | | |
| 33 | 2-16:0 | Lyso | LPS | 200.2 ± | ns | 43.9 ± | 0.0011 | 5 (3) |
| | LYSO PC | | | 98.2 | | 4.9 | | |
| 34 | 16:0 LYSO | Lyso | LPS | 199.5 ± | ns | 44.7 ± | 0.0003 | 5 (3) |
| | PC | | | 103.0 | | 4.3 | | |
| 35 | 17:0 LYSO | Lyso | LPS | 247.2 ± | ns | 40.0 ± | 0.0002 | 5 (3) |
| | PC | | | 117.3 | | 3.3 | | |
| 36 | 17:1 Δ¹⁰ | Lyso | LPS | 78.6 ± | ns | 95.3 ± | ns | 5 (3) |
| | LYSO PC | | | 79.6 | | 4.5 | | |
| 37 | 2-18:1 Δ9 | Lyso | LPS | 70.9 ± | ns | 97.7 ± | ns | 5 (3) |
| | LYSO PC | | | 59.7 | | 3.0 | | |
| 38 | 2-18:0 | Lyso | LPS | 289.4 ± | 0.0177 | 11.5 ± | 0.0004 | 5 (3) |
| | LYSO PC | | | 142.2 | | 8.6 | | |
| 39 | 18:0 LYSO | Lyso | LPS | 369.2 ± | 0.0002 | 12.2 ± | 0.0007 | 5 (3) |
| | PC | | | 200.3 | | 7.8 | | |
| 40 | 19:0 LYSO | Lyso | LPS | 209.3 ± | ns | 2.7 ± | <0.0001 | 5 (3) |
| | PC | | | 108.7 | | 1.7 | | |
| 41 | 20:0 LYSO | Lyso | LPS | 383.7 ± | <0.0001 | 6.0 ± | 0.0001 | 5 (3) |
| | PC | | | 193.5 | | 4.7 | | |
| 42 | 22:0 LYSO | Lyso | LPS | 2130.9 ± | <0.0001 | 14.6 ± | <0.0001 | 4 (3) |
| | PC | | | 857.5 | | 2.7 | | |
| 43 | 24:0 LYSO | Lyso | LPS | 39.7 ± | ns | 96.0 ± | ns | 5 (3) |
| | PC | | | 21.1 | | 5.6 | | |
| 44 | 26:0 LYSO | Lyso | LPS | 43.9 ± | ns | 94.7 ± | ns | 5 (3) |
| | PC | | | 27.2 | | 3.1 | | |
| 45 | PGPC | Oxidized | LPS | 271.6 ± | 0.0404 | 58.6 ± | 0.0093 | 5 (3) |
| | | | | 223.8 | | 11.9 | | |
| 46 | POVPC | Oxidized | LPS | 168.5 ± | ns | 79.5 ± | ns | 5 (3) |
| | | | | 156.0 | | 12.2 | | |
| 47 | oxPAPC | Oxidized | LPS | 78.3 ± | ns | 80.6 ± | 0.0002 | 5 (3) |
| | | | | 78.2 | | 2.0 | | |
| 48 | PAzePC | Oxidized | LPS | 153.4 ± | ns | 78.8 ± | 0.0062 | 5 (3) |
| | | | | 123.8 | | 5.5 | | |
| 49 | No Lipid | None | LPS | 31.1 ± | — | 100.0 ± | — | 5 (9) |
| | | | | 16.9 | | 0.0 | | |
| 50 | No Lipid | None | None | 0.0 ± | ns | 91.7 ± | ns | 5 (9) |
| | | | | 0.0 | | 20.0 | | |

\hat{ }IL-1β secretion is shown in pg/mL ± SD.
P values reflect the significance from LPS.
ns = not significant.

### Results—22:0 Lyso PC Has Potent Hyperstimulatory Ability

The initial screen for hyperactivating lipids was conducted using a lipid concentration of 82.5 μM with moDCs obtained from 5 human donors. However, different lipid species may have hyperactivating effects across a range of concentrations. Specifically, some of the lipids tested at 82.5 μM caused a decrease in cell viability, indicating that a lower lipid concentration may be optimal for maintaining cell viability while promoting IL-1β secretion. The lipid library was re-screened at 41.3 μM using moDCs obtained from 3 human donors. The results are shown in Table 1-2.

TABLE 1-2
IL-1β Secretion and Relative Viability (41.3 μM Lipid Treatment)\hat{ }

| | | | | | | Relative | | No. Donors |
|---|---|---|---|---|---|---|---|---|
| | | | | IL-1β | p- | Viability | p- | (Replicates) |
| Test | Lipid | Class | PAMP | secretion | value | (%) ± SD | value | Tested |
| 1 | 15:0-18:1 | Mixed | LPS | 42.3 ± | ns* | 94.6 ± | ns* | 3 (3) |
| | PC | Acyl | | 8.0 | | 0.8 | | |
| 2 | 14:0-16:0 | Mixed | LPS | 32.7 ± | ns | 97.1 ± | ns | 3 (3) |
| | PC | Acyl | | 3.0 | | 3.2 | | |
| 3 | 14:0-18:0 | Mixed | LPS | 24.0 ± | ns | 100.0 ± | ns | 3 (3) |
| | PC | Acyl | | 5.8 | | 1.7 | | |
| 4 | 16:0-14:0 | Mixed | LPS | 42.2 ± | ns | 101.0 ± | ns | 3 (3) |
| | PC | Acyl | | 5.8 | | 0.8 | | |
| 5 | 16:0-18:0 | Mixed | LPS | 26.3 ± | ns | 97.7 ± | ns | 3 (3) |
| | PC | Acyl | | 7.4 | | 1.8 | | |
| 6 | 16:0-18:1 | Mixed | LPS | 28.2 ± | ns | 96.5 ± | ns | 3 (3) |
| | PC (POPC) | Acyl | | 5.2 | | 5.4 | | |
| 7 | 16:0-18:2 | Mixed | LPS | 31.5 ± | ns | 99.0 ± | ns | 3 (3) |
| | PC | Acyl | | 7.8 | | 4.8 | | |
| 10 | 18:0-14:0 | Mixed | LPS | 31.2 ± | ns | 98.1 ± | ns | 3 (3) |
| | PC | Acyl | | 3.5 | | 2.1 | | |
| 11 | 18:0-16:0 | Mixed | LPS | 20.1 ± | ns | 100.3 ± | ns | 3 (3) |
| | PC | Acyl | | 5.3 | | 3.1 | | |
| 12 | 18:0-18:1 | Mixed | LPS | 23.5 ± | ns | 99.7 ± | ns | 3 (3) |
| | PC | Acyl | | 5.3 | | 1.7 | | |
| 13 | 18:0-18:2 | Mixed | LPS | 22.6 ± | ns | 102.4 ± | ns | 3 (3) |
| | PC | Acyl | | 8.1 | | 1.4 | | |
| 14 | 18:0-20:4 | Mixed | LPS | 38.8 ± | ns | 91.8 ± | ns | 3 (3) |
| | PC | Acyl | | 4.8 | | 1.3 | | |
| 16 | 18:1-14:0 | Mixed | LPS | 34.5 ± | ns | 101.1 ± | ns | 3 (3) |
| | PC | Acyl | | 1.5 | | 1.5 | | |
| 17 | 18:1-16:0 | Mixed | LPS | 36.4 ± | ns | 94.3 ± | ns | 3 (3) |
| | PC | Acyl | | 5.0 | | 1.4 | | |
| 18 | 18:1-18:0 | Mixed | LPS | 32.2 ± | ns | 99.1 ± | ns | 3 (3) |
| | PC | Acyl | | 9.4 | | 3.9 | | |
| 19 | 18:1(n10)- | Mixed | LPS | 31.0 ± | ns | 100.4 ± | ns | 3 (3) |
| | 16:0 PC | Acyl | | 3.0 | | 2.5 | | |
| 21 | 16:0-(12- | Mixed | LPS | 37.0 ± | ns | 98.3 ± | ns | 3 (3) |
| | PAHSA) PC | Acyl | | 10.8 | | 0.3 | | |
| 22 | 18:1 Δ⁹ | Lyso | LPS | 34.8 ± | ns | 97.8 ± | ns | 3 (3) |
| | LYSO PC | | | 3.5 | | 3.3 | | |
| 23 | 06:0 LYSO | Lyso | LPS | 48.2 ± | ns | 96.1 ± | ns | 3 (3) |
| | PC | | | 16.0 | | 1.9 | | |
| 24 | 07:0 LYSO | Lyso | LPS | 36.0 ± | ns | 97.9 ± | ns | 3 (3) |
| | PC | | | 14.9 | | 3.4 | | |
| 25 | 08:0 LYSO | Lyso | LPS | 32.2 ± | ns | 96.7 ± | ns | 3 (3) |
| | PC | | | 10.3 | | 2.2 | | |
| 26 | 09:0 LYSO | Lyso | LPS | 31.3 ± | ns | 98.2 ± | ns | 3 (3) |
| | PC | | | 12.6 | | 2.6 | | |
| 27 | 10:0 LYSO | Lyso | LPS | 33.3 ± | ns | 97.8 ± | ns | 3 (3) |
| | PC | | | 11.6 | | 2.9 | | |
| 28 | 11:0 LYSO | Lyso | LPS | 39.3 ± | ns | 94.9 ± | ns | 3 (3) |
| | PC | | | 11.3 | | 4.2 | | |
| 29 | 12:0 LYSO | Lyso | LPS | 54.5 ± | ns | 96.6 ± | ns | 3 (3) |
| | PC | | | 13.8 | | 5.0 | | |
| 30 | 13:0 LYSO | Lyso | LPS | 80.7 ± | ns | 93.9 ± | ns | 3 (3) |
| | PC | | | 37.0 | | 6.8 | | |
| 31 | 14:0 LYSO | Lyso | LPS | 160.0 ± | <0.0001 | 90.7 ± | ns | 3 (3) |
| | PC | | | 89.3 | | 3.1 | | |
| 32 | 15:0 LYSO | Lyso | LPS | 150.3 ± | <0.0001 | 90.8 ± | ns | 3 (3) |
| | PC | | | 60.6 | | 1.0 | | |
| 33 | 2-16:0 | Lyso | LPS | 93.0 ± | ns | 87.2 ± | ns | 3 (3) |
| | LYSO PC | | | 27.2 | | 1.2 | | |
| 34 | 16:0 LYSO | Lyso | LPS | 97.4 ± | ns | 87.2 ± | 0.0196 | 3 (3) |
| | PC | | | 30.5 | | 0.3 | | |
| 35 | 17:0 LYSO | Lyso | LPS | 106.1 ± | 0.0170 | 85.2 ± | ns | 3 (3) |
| | PC | | | 13.3 | | 0.8 | | |
| 36 | 17:1 Δ¹⁰ | Lyso | LPS | 42.7 ± | ns | 97.2 ± | ns | 3 (3) |
| | LYSO PC | | | 13.7 | | 3.6 | | |
| 37 | 2-18:1 Δ⁹ | Lyso | LPS | 45.0 ± | ns | 100.6 ± | ns | 3 (3) |
| | LYSO PC | | | 9.8 | | 3.2 | | |
| 38 | 2-18:0 | Lyso | LPS | 225.0 ± | <0.0001 | 65.3 ± | ns | 3 (3) |
| | LYSO PC | | | 18.9 | | 2.0 | | |
| 39 | 18:0 LYSO | Lyso | LPS | 239.5 ± | <0.0001 | 69.9 ± | ns | 3 (3) |
| | PC | | | 39.6 | | 7.6 | | |
| 40 | 19:0 LYSO | Lyso | LPS | 468.9 ± | <0.0001 | 39.4 ± | 0.0335 | 3 (3) |
| | PC | | | 105.1 | | 2.6 | | |
| 41 | 20:0 LYSO | Lyso | LPS | 168.7 ± | <0.0001 | 75.2 ± | ns | 3 (3) |
| | PC | | | 37.2 | | 5.0 | | |
| 42 | 22:0 LYSO | Lyso | LPS | 1904.2 ± | <0.0001 | 47.6 ± | ns | 3 (3) |
| | PC | | | 0.0 | | 8.8 | | |
| 43 | 24:0 LYSO | Lyso | LPS | 38.8 ± | ns | 94.6 ± | ns | 3 (3) |
| | PC | | | 6.0 | | 2.8 | | |
| 44 | 26:0 LYSO | Lyso | LPS | 43.1 ± | ns | 93.3 ± | ns | 3 (3) |
| | PC | | | 12.8 | | 1.6 | | |
| 45 | PGPC | Oxidized | LPS | 132.9 ± | 0.0017 | 87.2 ± | ns | 2 (3) |
| | | | | 38.4 | | 1.2 | | |
| 46 | POVPC | Oxidized | LPS | 121.5 ± | 0.0104 | 92.1 ± | ns | 2 (3) |
| | | | | 43.8 | | 2.8 | | |
| 47 | oxPAPC | Oxidized | LPS | 83.0 ± | ns | 84.4 ± | ns | 2 (3) |
| | | | | 40.0 | | 0.8 | | |
| 48 | PAzePC | Oxidized | LPS | 191.7 ± | <0.0001 | 95.6 ± | ns | 2 (3) |
| | | | | 105.0 | | 2.1 | | |
| 49 | No Lipid | — | LPS | 34.6 ± | — | 100.0 ± | — | 3 (8) |
| | | | | 7.8 | | 0.0 | | |
| 50 | No Lipid | — | None | 0.0 ± | ns | 98.8 ± | ns | 3 (8) |
| | | | | 0.0 | | 18.2 | | |

\hat{ }IL-1β secretion is shown in pg/mL ± SD.
P values reflect the significance from LPS.
ns = not significant.

At 41.3 μM, the lipids that induced IL-1β secretion largely confirmed results observed at 82.5 μM (Table 1-2, compared to Table 1). As hypothesized, viability of moDCs treated with lipids that induced IL-1β secretion was improved at 41.3 μM compared to 82.5 μM (Table 1-2).

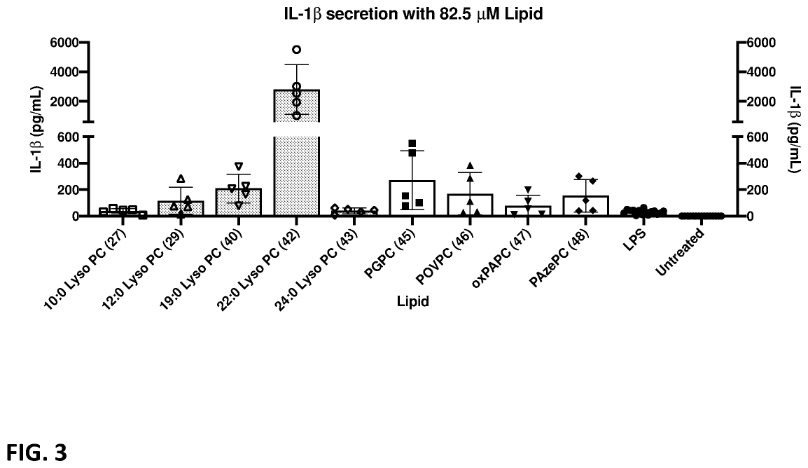

Several lipids were then selected for use in additional studies (Table 1-3 and FIG. 3). From the LYSO PC subset, 22:0 LYSO PC and 19:0 LYSO PC were chosen for additional study based on their robust induction of IL-1β secretion. Additionally, 12:0 LYSO PC was chosen because it was the smallest acyl chain lipid to stimulate IL-1β secretion. As negative control lipids, 10:0 LYSO PC and 24:0 Lyso PC were chosen since the acyl chain lengths were just outside the range of IL-1β secretion activity. From the oxidized lipid group, PGPC, POVPC, and oxPAPC were kept for further study, as the screens were based on studies including these lipids. PAzePC was selected for further study because it promoted IL-1β secretion, was structurally similar to the other oxidized lipids, and had the longest second acyl chain tested while still retaining activity. Results for the selected lipids are shown in Table 1-3 and FIG. 3, in order to more clearly demonstrate the differences observed in these active and inactive lipid species.

TABLE 1-3
IL-1β Secretion and Relative Viability (82.5 μM Lipid Treatment)\hat{ }

| | | | | | | Relative | | No. Donors |
|---|---|---|---|---|---|---|---|---|
| | | | | IL-1β | p- | Viability | p- | (Replicates) |
| Test | Lipid | Class | PAMP | secretion | value | (%) ± SD | value | Tested |
| 27 | 10:0 | Lyso | LPS | 37.8 ± | ns | 95.2 ± | ns | 5 (3) |
| | LYSO PC | | | 19.2 | | 4.2 | | |
| 29 | 12:0 | Lyso | LPS | 115.3 ± | ns | 91.1 ± | ns | 5 (3) |
| | LYSO PC | | | 103.0 | | 5.5 | | |
| 40 | 19:0 | Lyso | LPS | 209.3 ± | ns | 2.7 ± | <0.0001 | 5 (3) |
| | LYSO PC | | | 108.7 | | 1.7 | | |
| 42 | 22:0 | Lyso | LPS | 2130.9 ± | <0.0001 | 14.6 ± | <0.0001 | 4 (3) |
| | LYSO PC | | | 857.5 | | 2.7 | | |
| 43 | 24:0 | Lyso | LPS | 39.7 ± | ns | 96.0 ± | ns | 5 (3) |
| | LYSO PC | | | 21.1 | | 5.6 | | |
| 45 | PGPC | Oxidized | LPS | 271.6 ± | 0.0404 | 58.6 ± | 0.0093 | 5 (3) |
| | | | | 223.8 | | 11.9 | | |
| 46 | POVPC | Oxidized | LPS | 168.5 ± | ns | 79.5 ± | ns | 5 (3) |
| | | | | 156.0 | | 12.2 | | |
| 47 | oxPAPC | Oxidized | LPS | 78.3 ± | ns | 80.6 ± | 0.0002 | 5 (3) |
| | | | | 78.2 | | 2.0 | | |
| 48 | PAzePC | Oxidized | LPS | 153.4 ± | ns | 78.8 ± | 0.0062 | 5 (3) |
| | | | | 123.8 | | 5.5 | | |
| 49 | No Lipid | — | LPS | 31.1 ± | — | 100.0 ± | — | 5 (9) |
| | | | | 16.9 | | 0.0 | | |
| 50 | No Lipid | — | None | 0.0 ± | ns | 91.7 ± | ns | 5 (9) |
| | | | | 0.0 | | 20.0 | | |

\hat{ }IL-1β secretion is shown in pg/mL ± SD.
P values reflect the significance from LPS.
ns = not significant.

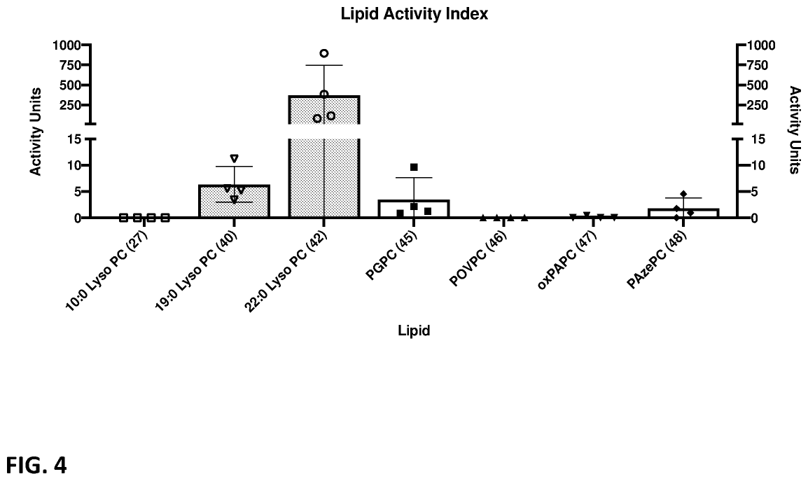

IL-1β secretion and cell viability were studied on a wider range of lipid concentrations to further understand the potency of hyperactivating lipids. moDCs obtained from 4 human donors were activated with LPS. Lipids of interest were added to cells at 82.5 μM, with 2-fold dilutions in concentration to a final tested concentration of 1.3 μM. The potencies of each lipid were compared, with lipid activity indices calculated by multiplying the reciprocal of the lowest concentration that induced IL-1β secretion at levels two-fold higher than cells treated with LPS by the highest IL-1β signal observed at any concentration. Results for the selected lipids are shown in Table 1-4 and FIG. 4. In summary, 22:0 Lyso PC had the most potent activity by far, followed by 19:0 Lyso PC. PGPC and PAzePC had similar activity indices.

TABLE 1-4
Activity Indices

| | | | | Activity Units | Donors (Replicates) |
|---|---|---|---|---|---|
| Test | Lipid | Class | PAMP | (AU) ± SD | Tested |
| 27 | 10:0 LYSO PC | Lyso | LPS | 0.00 ± 0.00 | 4 (3) |
| 40 | 19:0 LYSO PC | Lyso | LPS | 6.34 ± 3.39 | 4 (3) |
| 42 | 22:0 LYSO PC | Lyso | LPS | 370.00 ± 376.36 | 1 (3) |
| 45 | PGPC | Oxidized | LPS | 3.46 ± 4.14 | 4 (3) |
| 46 | POVPC | Oxidized | LPS | 0.00 ± 0.00 | 4 (3) |
| 47 | oxPAPC | Oxidized | LPS | 0.11 ± 0.22 | 4 (3) |
| 48 | PAzePC | Oxidized | LPS | 1.81 ± 1.95 | 4 (3) |

Titrating the lipids also extended our knowledge on the activity of the selected lipids. At 10.3 μM, 22:0 Lyso PC retained its ability to induce IL-1beta secretion while all other lipids tested could not (Table 1-5). At 10.3 μM, moDCs treated with 22:0 Lyso PC retained high viability (Table 1-5), thus demonstrating both characteristics of hyperactivated dendritic cells. At 20.6 μM, 22:0 Lyso PC increased IL-1beta secretion whereas the other lipids tested did not (Table 1-6), although this reduced cell viability somewhat.

TABLE 1-5
IL-1β Secretion and Relative Viability (10.3 μM Lipid Treatment)\hat{ }

| | | | | | Relative | | Donors |
|---|---|---|---|---|---|---|---|
| | | | IL-1β | p- | Viability | p- | (Replicates) |
| Test | Lipid | PAMP | secretion | value | (%) ± SD | value | Tested |
| 27 | 10:0 LYSO | LPS | 35.7 ± | ns* | 100.5 ± | ns | 4 (3) |
| | PC | | 28.3 | | 6.9 | | |
| 29 | 12:0 LYSO | LPS | 43.1 ± | ns | 97.3 ± | ns | 4 (3) |
| | PC | | 39.5 | | 4.7 | | |
| 40 | 19:0 LYSO | LPS | 43.0 ± | ns | 96.4 ± | ns | 4 (3) |
| | PC | | 31.9 | | 4.5 | | |
| 42 | 22:0 LYSO | LPS | 224.8 ± | ns | 89.1 ± | ns | 4 (3) |
| | PC | | 211.2 | | 4.1 | | |
| 43 | 24:0 LYSO | LPS | 36.5 ± | ns | 96.8 ± | ns | 4 (3) |
| | PC | | 34.1 | | 6.6 | | |
| 45 | PGPC | LPS | 52.8 ± | ns | 98.5 ± | ns | 4 (3) |
| | | | 51.1 | | 4.1 | | |
| 46 | POVPC | LPS | 49.1 ± | ns | 97.3 ± | ns | 4 (3) |
| | | | 43.1 | | 4.2 | | |
| 47 | oxPAPC | LPS | 46.4 ± | ns | 92.4 ± | 0.0246 | 4 (3) |
| | | | 40.6 | | 2.2 | | |
| 48 | PAzePC | LPS | 47.9 ± | ns | 98.0 ± | ns | 4 (3) |
| | | | 41.3 | | 1.3 | | |
| 49 | No Lipid | LPS | 42.8 ± | — | 100.0 ± | — | 4 (9) |
| | | | 35.8 | | 0.0 | | |

\hat{ }IL-1β secretion is shown in pg/mL ± SD.
P values reflect the significance from LPS.
ns = not significant.

TABLE 1-6
IL-1β Secretion and Relative Viability (20.6 μM Lipid Treatment)\hat{ }

| | | | | | Relative | | Donors |
|---|---|---|---|---|---|---|---|
| | | | IL-1β | p- | Viability | p- | (Replicates) |
| Test | Lipid | PAMP | secretion | value | (%) ± SD | value | Tested |
| 27 | 10:0 LYSO | LPS | 35.2 ± | ns* | 100.4 ± | ns | 4 (3) |
| | PC | | 28.8 | | 4.8 | | |
| 29 | 12:0 LYSO | LPS | 44.2 ± | ns | 96.6 ± | ns | 4 (3) |
| | PC | | 39.8 | | 3.4 | | |
| 40 | 19:0 LYSO | LPS | 55.6 ± | ns | 94.6 ± | ns | 4 (3) |
| | PC | | 34.1 | | 5.0 | | |
| 42 | 22:0 LYSO | LPS | 512.6 ± | ns | 79.2 ± | 0.0209 | 4 (3) |
| | PC | | 485.9 | | 5.7 | | |
| 43 | 24:0 LYSO | LPS | 36.6 ± | ns | 97.3 ± | ns | 4 (3) |
| | PC | | 32.7 | | 4.0 | | |
| 45 | PGPC | LPS | 69.2 ± | ns | 98.0 ± | ns | 4 (3) |
| | | | 68.1 | | 3.9 | | |
| 46 | POVPC | LPS | 60.6 ± | ns | 98.1 ± | ns | 4 (3) |
| | | | 55.9 | | 1.5 | | |
| 47 | oxPAPC | LPS | 34.4 ± | ns | 91.6 ± | ns | 4 (3) |
| | | | 39.7 | | 5.4 | | |
| 48 | PAzePC | LPS | 59.0 ± | ns | 96.0 ± | 0.0310 | 4 (3) |
| | | | 53.3 | | 1.3 | | |
| 49 | No Lipid | LPS | 42.8 ± | — | 100.0 ± | — | 4 (9) |
| | | | 35.8 | | 0.0 | | |

\hat{ }IL-1β secretion is shown in pg/mL ± SD.
P values reflect the significance from LPS.
ns = not significant.

At 10.3 μM, 22:0 Lyso PC stimulates IL-1beta secretion at a comparable concentration to PGPC at 82.5 μM (Table 1-5 and Table 1-1). Thus, 22:0 Lyso PC can be used at a concentration nearly 8 times lower than PGPC, making 22:0 Lyso PC the most potent hyperactivating lipid identified to date.

### Results—Innate Immune Signaling Pathway Agonists can Hyperactivate Dendritic Cells

Previous experiments indicated that two signals are required to hyperactivate dendritic cells: an innate immune agonist (e.g., PAMP such as LPS) to produce the immature form of IL-1β, and a lipid (e.g., DAMP such as PGPC) to enable IL-1β secretion. Although the DAMP screens described above used LPS as the innate immune agonist, LPS is not used as a clinical agonist due to toxicity concerns. Thus, a selection of clinically relevant innate immune signaling pathway agonists were tested in combination with 22:0 Lyso PC for the ability to hyperactivate human moDCs.

The PRR agonists tested predominantly focused on TLR signaling pathway agonists, although some cGAS/STING signaling pathway agonists and a C-type lectin family receptor agonist were also tested. moDC from ten different human donors were used to initially test the agonists (Table 1-7). moDCs from five of the ten human donors were also used to test the agonists at a lower concentration (Table 1-8). PAMPs were added to moDC culture following differentiation, along with the DAMP, 22:0 Lyso PC. Cells were stimulated for 2 days before measuring cell viability and IL-1β secretion.

TABLE 1-7
IL-1β Secretion and Relative Viability (High Dose PAMP Treatment)\hat{ }

| | | | | | | Relative | | Donors |
|---|---|---|---|---|---|---|---|---|
| | PAMP | PAMP | DAMP | IL-1β | p- | Viability | p- | (Replicates) |
| Test | Dose | Class | (20 μM) | secretion | value | (%) ± SD | value | Tested |
| 1 | Poly(A:U) | TLR3 | + | 0.4 ± | ns | 93.9 ± | 0.0064 | 10 (3) |
| | 100 μg/mL | agonist | | 1.4 | | 13.4 | | |
| 2 | Poly(I:C) | TLR3 | + | 0.6 ± | ns | 106.9 ± | ns | 10 (3) |
| | HMW | agonist | | 1.7 | | 7.7 | | |
| | 10 μg/mL | | | | | | | |
| 3 | Poly(I:C) | TLR3 | + | 0.4 ± | ns | 108.7 ± | ns | 10 (3) |
| | LMW | agonist | | 1.3 | | 8.2 | | |
| | 10 μg/mL | | | | | | | |
| 4 | Imiquimod | TLR7 | + | 1.4 ± | ns | 108.8 ± | ns | 10 (3) |
| | 5 μg/mL | agonist | | 2.5 | | 5.4 | | |
| 5 | R848 | TLR7/8 | + | 342.1 ± | 0.0033 | 82.7 ± | 0.0024 | 10 (3) |
| | 10 μg/mL | agonist | | 210.5 | | 11.4 | | |
| 6 | Loxoribine | TLR7 | + | 0.5 ± | ns | 66.3 ± | <0.0001 | 10 (3) |
| | 300 μg/mL | agonist | | 1.6 | | 13.5 | | |
| 7 | TL8-506 | TLR8 | + | 47.2 ± | ns | 111.5 ± | ns | 10 (3) |
| | 100 ng/ml | agonist | | 44.9 | | 6.9 | | |
| 8 | 2′3′-cGAMP | STING | + | 0.4 ± | ns | 105.6 ± | ns | 10 (3) |
| | 15 μg/mL | agonist | | 1.2 | | 9.2 | | |
| 9 | c-di-AMP | STING | + | 1.4 ± | ns | 99.8 ± | 0.0365 | 10 (3) |
| | 20 μg/mL | Agonist | | 3.5 | | 12.9 | | |
| 10 | c-di-GMP | STING | + | 0.3 ± | ns | 107.4 ± | 0.0335 | 10 (3) |
| | 20 μg/mL | agonist | | 0.8 | | 7.6 | | |
| 11 | ORN Sa19 | TLR13/8 | + | 0.0 ± | ns | 104.8 ± | 0.0192 | 10 (3) |
| | 2 μg/mL | agonist | | 0.0 | | 8.2 | | |
| 12 | TDB | Mincle | + | 0.0 ± | ns | 87.0 ± | 0.0002 | 10 (3) |
| | 100 μg/mL | agonist | | 0.0 | | 14.0 | | |
| 13 | TDM | Mincle | + | 2.0 ± | ns | 95.2 ± | 0.0022 | 10 (3) |
| | 10 μg/mL | agonist | | 6.4 | | 14.1 | | |
| 14 | Pam3CSK4 | TLR2/1 | + | 0.5 ± | ns | 125.9 ± | ns | 10 (3) |
| | 10 ng/ml | agonist | | 1.1 | | 13.2 | | |
| 15 | Pam2CSK4 | TLR2/6 | + | 165.5 ± | 0.0171 | 102.4 ± | 0.0220 | 10 (3) |
| | 100 ng/mL | agonist | | 129.8 | | 5.9 | | |
| 16 | ODN 2336 | TLR9 | + | 91.5 ± | 0.0128 | 116.5 ± | ns | 10 (3) |
| | 5 μM | agonist | | 68.2 | | 12.5 | | |
| 17 | ODN 2006- | TLR9 | + | 0.6 ± | ns | 111.5 ± | ns | 10 (3) |
| | G5 | agonist | | 2.0 | | 12.1 | | |
| | 5 μM | | | | | | | |
| 18 | MPLA-SM | TLR4 | + | 0.0 ± | ns | 126.6 ± | ns | 10 (3) |
| | 1 μg/mL | agonist | | 0.0 | | 18.7 | | |
| 19 | LPS + QuilA | TLR4 | + | 556.0 ± | 0.0015 | 51.0 ± | <0.0001 | 10 (3) |
| | 1 μg/mL + | agonist + | | 307.9 | | 8.8 | | |
| | 10 μM | adjuvant | | | | | | |
| 20 | LPS + Alum | TLR4 | + | 625.2 ± | 0.0020 | 72.5 ± | <0.0001 | 10 (3) |
| | 1 μg/mL + | agonist + | | 359.4 | | 10.5 | | |
| | 300 μg/mL | adjuvant | | | | | | |
| 21 | LPS | TLR4 | + | 953.1 ± | <0.0001 | 88.2 ± | <0.0001 | 10 (6) |
| | 1 μg/mL | agonist | | 594.1 | | 6.8 | | |
| 22 | LPS | TLR4 | None | 54.7 ± | 0.0011 | 100.0 ± | <0.0001 | 10 (6) |
| | 1 μg/mL | agonist | | 46.4 | | 0.0 | | |
| 23 | No PAMP | None | + | 1.1 ± | — | 119.7 ± | — | 10 (6) |
| | | | | 3.4 | | 11.3 | | |
| 24 | No PAMP | None | None | 3.4 ± | ns | 115.3 ± | ns | 10 (6) |
| | | | | 5.7 | | 12.2 | | |

\hat{ }DAMP = 22:0 Lyso PC.
IL-1β secretion is shown in pg/mL ± SD.
P values reflect the significance from LPS.
ns = not significant.

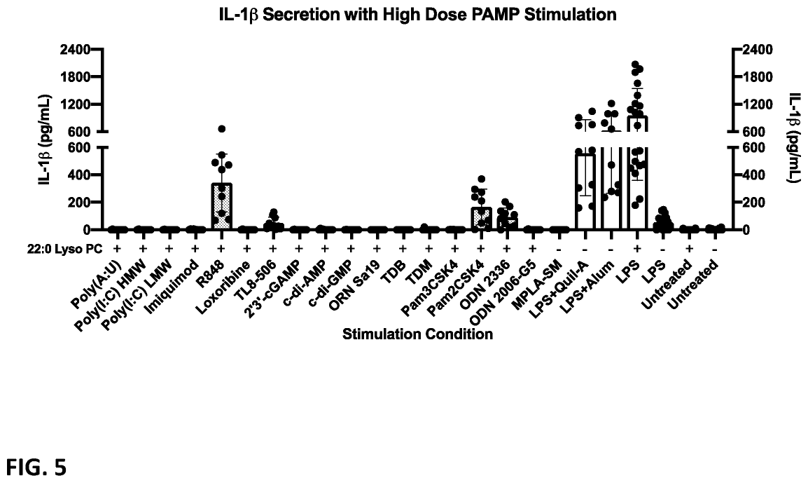

Table 7 and FIG. 5 indicate that four agonists enabled IL-1β secretion: R848, TL8-506, Pam2CSK4, and ODN 2336. These agonists had little to no effect on cell viability (Table 7). Table 8 shows the results of the low dose PAMP treatment.

TABLE 1-8
IL-1β Secretion and Relative Viability (Low Dose PAMP Treatment)\hat{ }

| | | | | | | Relative | | Donors |
|---|---|---|---|---|---|---|---|---|
| | PAMP | PAMP | DAMP | IL-1β | p- | Viability | p- | (Replicates) |
| Test | Dose | Class | (20 μM) | secretion | value | (%) ± SD | value | Tested |
| 1 | Poly(A:U) | TLR3 | + | 0.0 ± | ns | 95.8 ± | ns | 5 (3) |
| | 30 μg/mL | agonist | | 0.0 | | 19.2 | | |
| 2 | Poly(I:C) | TLR3 | + | 3.0 ± | ns | 103.3 ± | 0.0285 | 5 (3) |
| | HMW | agonist | | 6.7 | | 7.9 | | |
| | 3 μg/mL | | | | | | | |
| 3 | Poly(I:C) | TLR3 | + | 0.0 ± | ns | 105.1 ± | ns | 5 (3) |
| | LMW | agonist | | 0.0 | | 7.4 | | |
| | 3 μg/mL | | | | | | | |
| 4 | Imiquimod | TLR7 | + | 1.8 ± | ns | 109.9 ± | ns | 5 (3) |
| | 1.7 μg/mL | agonist | | 2.7 | | 6.5 | | |
| 5 | R848 | TLR7/8 | + | 326.2 ± | ns | 96.1 ± | ns | 5 (3) |
| | 3 μg/mL | agonist | | 213.1 | | 8.0 | | |
| 6 | Loxoribine | TLR7 | + | 0.0 ± | ns | 95.0 ± | 0.0092 | 5 (3) |
| | 100 μg/mL | agonist | | 0.0 | | 8.7 | | |
| 7 | TL8-506 | TLR8 | + | 0.6 ± | ns | 109.3 ± | ns | 5 (3) |
| | 30 ng/ml | agonist | | 1.3 | | 4.6 | | |
| 8 | 2′3′- | STING | + | 7.3 ± | ns | 109.8 ± | ns | 5 (3) |
| | cGAMP | agonist | | 8.3 | | 3.2 | | |
| | 10 μg/mL | | | | | | | |
| 9 | c-di-AMP | STING | + | 0.0 ± | ns | 104.3 ± | ns | 5 (3) |
| | 10 μg/mL | Agonist | | 0.0 | | 16.0 | | |
| 10 | c-di-GMP | STING | + | 0.0 ± | ns | 106.3 ± | ns | 5 (3) |
| | 10 μg/mL | agonist | | 0.0 | | 7.7 | | |
| 11 | ORN Sa19 | TLR13/8 | + | 0.0 ± | ns | 105.9 ± | ns | 5 (3) |
| | 0.7 μg/mL | agonist | | 0.0 | | 11.2 | | |
| 12 | TDB | Mincle | + | 0.0 ± | ns | 79.6 ± | 0.0014 | 5 (3) |
| | 30 μg/mL | agonist | | 0.0 | | 4.5 | | |
| 13 | TDM | Mincle | + | 0.0 ± | ns | 107.7 ± | ns | 5 (3) |
| | 3 μg/mL | agonist | | 0.0 | | 12.7 | | |
| 14 | Pam3CSK4 | TLR2/1 | + | 0.0 ± | ns | 132.3 ± | 0.0484 | 5 (3) |
| | 3 ng/mL | agonist | | 0.0 | | 7.2 | | |
| 15 | Pam2CSK4 | TLR2/6 | + | 147.0 ± | ns | 103.5 ± | ns | 5 (3) |
| | 30 ng/ml | agonist | | 136.0 | | 9.4 | | |
| 16 | ODN 2336 | TLR9 | + | 0.0 ± | ns | 110.2 ± | ns | 5 (3) |
| | 1.7 μM | agonist | | 0.0 | | 13.0 | | |
| 17 | ODN 2006- | TLR9 | + | 0.0 ± | ns | 112.9 ± | ns | 5 (3) |
| | G5 | agonist | | 0.0 | | 12.1 | | |
| | 1.7 μM | | | | | | | |
| 18 | MPLA-SM | TLR4 | + | 0.0 ± | ns | 133.5 ± | ns | 5 (3) |
| | 0.3 μg/mL | agonist | | 0.0 | | 13.9 | | |
| 19 | LPS + QuilA | TLR4 | + | 443.8 ± | ns | 58.2 ± | 0.0002 | 10 (3) |
| | 1 μg/mL + | agonist + | | 341.2 | | 6.9 | | |
| | 3 μM | adjuvant | | | | | | |
| 20 | LPS + Alum | TLR4 | + | 453.1 ± | ns | 80.4 ± | 0.0012 | 10 (3) |
| | 1 μg/mL + | agonist + | | 247.3 | | 5.1 | | |
| | 100 μg/mL | adjuvant | | | | | | |
| 21 | LPS | TLR4 | + | 845.8 ± | 0.0288 | 88.2 ± | <0.0001 | 10 (6) |
| | 1 μg/mL | agonist | | 629.1 | | 6.8 | | |
| 22 | LPS | TLR4 | None | 53.3 ± | ns | 100.0 ± | <0.0001 | 10 (6) |
| | 1 μg/mL | agonist | | 55.8 | | 0.0 | | |
| 23 | No PAMP | None | + | 1.0 ± | — | 119.7 ± | — | 10 (6) |
| | | | | 3.2 | | 11.3 | | |
| 24 | No PAMP | None | None | 4.5 ± | ns | 115.3 ± | ns | 10 (6) |
| | | | | 6.5 | | 12.2 | | |

\hat{ }DAMP = 22:0 Lyso PC.
IL-1β secretion is shown in pg/mL ± SD.
P values reflect the significance from LPS.
ns = not significant.

As shown in Table 1-7 and FIG. 5, four agonists (R848, TL8-506, Pam2CSK4, and ODN 2336) induced IL-1β secretion by human DCs in the presence of 22:0 Lyso PC. These PRR agonists had little to no effect on cell viability (Table 1-7). At the lower agonist concentration tested, IL-1β secretion was only observed when R848 or Pam2CSK4 were used in combination with 22:0 Lyso PC (Table 1-8). Neither R848 nor Pam2CSK4 at the lower concentrations tested affected cell viability (Table 1-8). From this small screen of clinically relevant innate immune signaling pathway agonists, only a handful were found to be capable of hyperactivating human moDCs when combined with 22:0 Lyso PC. Based on IL-1β secretion, R848 is the most potent clinically-relevant candidate as an agonist that promotes human DC hyperactivation (e.g., secrete IL-1β and maintain cell viability).

### Example 2: Combination of a Lysophosphatidylcholine (LPC) with a Single Acyl Chain and a TLR7/8 Agonist Hyperactivates Mammalian Peripheral Blood Mononuclear Cells

This example describes the hyperactivation of canine and human peripheral blood mononuclear cells (PBMCs) with a lipid DAMP in combination with a small molecule PAMP.

### Materials and Methods

Isolation of PBMCs from Whole Blood. PBMCs were isolated from whole blood using density gradient centrifugation with Ficoll-Paque PLUS (Cytivia). Whole blood was diluted 1:1 with PBS, layered on top of Ficoll-Paque PLUS and centrifuged at 1000×g for 30 minutes at room temperature. PBMCs were collected, washed twice in PBS, and incubated with Ack lysis buffer (Lonza) to remove any remaining red blood cells.

Cell Culture and Stimulation. Immediately following isolation, PBMCs were plated in RPMI medium containing 10% FBS, 50 units/mL penicillin, 50 mg/mL streptomycin, 2 mM L-glutamine, 1 mM sodium pyruvate, and 50 mM beta-mercaptoethanol (R10 media). Cells were plated at 1×10⁵ (canine cells) or 1×10⁶ (human cells) per well in 96-well flat bottom tissue culture plates. Lyophilized Vaccigrade R848 (Invivogen) was reconstituted and diluted according to manufacturer's recommendations and added to cells at a final concentration of 1 μg/mL. Immediately following, 22:0 LYSO PC was added to cells at a final concentration of 82.5 μM. Additional innate agonists were diluted in R10 media according to manufacturer's recommendations and added to the cells as follows: human GM-CSF (Peprotech) was added at a final concentration of 10 ng/mL; 2′3′ cGAMP (Invivogen) was added at a final concentration of 15 μg/mL; LPS, serotype 055:B5 (Enzo Life Sciences) was added at a final concentration of 1 μg/mL; Alum hydroxide (Invivogen) was added at a final concentration of 30 μg/mL. Cells were incubated at 37° C., 5% CO₂ for two days. Cell cultures were then used for endpoint analyses.

Endpoint Analyses. After culturing PBMCs with PAMPs and DAMPs for two days, supernatant and cell samples were collected for analysis. Cells in culture were pelleted by centrifugation at 400×g for 5 minutes. Half of the media volume in the wells was collected for cytokine quantification by Enzyme-Linked Immunosorbent Assay (ELISA) or Lumit™ Bioluminescent assay, while the remaining media and cells were used to quantify cell viability by assessing metabolic activity.

Quantification of Cytokine Secretion. IL-1β secretion from human PBMCs was assessed using one of the following kits: ELISA MAX Deluxe Set Human IL-1β kit (Biolegend), Invitrogen Human IL-1β kit, or the Lumit™ Human IL-1β Immunoassay (Promega). IFNγ secretion from human PBMCs was assessed using the ELISA MAX Deluxe Set Human IFNγ (Biolegend) and TNFα secretion from human PBMCs was assessed using the Human TNFα Uncoated ELISA kit (Invitrogen). ELISAs were performed according to manufacturer's instructions with the following modifications: i) total sample+buffer volume for incubation was reduced from 100 μL to 50 μL; ii) the top standard was prepared at 500 μg/mL, with two-fold dilutions to 7.8 μg/mL; and iii) sample incubation was completed overnight at 4 C on an orbital shaker. Lumit™ assays were performed according to manufacturer's instructions. IL-1β secretion from canine PBMCs was assessed using the Canine IL-1β/IL-1F2 DuoSet ELISA (R&D) according to manufacturer's instructions with the following modifications: i) total sample+buffer volume for incubation was reduced from 100 μL to 50 μL; ii) sample incubation was completed overnight at 4° C. on an orbital shaker. For all ELISAs, absorbance was measured at 450 nm, with a 570 nm correction, using a Spectramax M5e plate reader (Molecular Devices). For Lumit™ assays, luminescence was measured on all wavelengths using a Spectramax M5e plate reader (Molecular Devices) with an integration time of 500 ms. To determine cytokine concentrations in supernatants, sample concentrations were interpolated using a standard curve via 4PL analysis on GraphPad Prism 9 (GraphPad Software). The interpolated results of samples were then adjusted for any dilutions made to the supernatant.

Quantification of Cell Viability. Cell viability was assessed by quantifying the presence of ATP as an indicator of metabolically active cells using the CellTiter-Glo Luminescent Cell Viability Assay (Promega). Metabolic activity was assessed following manufacturer's instructions. The CellTiter-Glo reagent was mixed with the cell pellets and fresh media then transferred to a white, opaque 96-well plate. Luminescence was measured on all wavelengths on a Spectramax M5e plate reader (Molecular Devices) using an integration time of 500 ms. Percent viability was calculated relative to the control condition of PBMCs treated with R848.

Statistical Analyses. For each condition, cells from each donor were plated for testing in triplicate. For cytokine quantification, triplicate values were used for interpolation and data was plotted as total concentration (pg/mL) or fold change per donor relative to the control condition of R848 alone. For viability quantification, each donor triplicate was averaged, and the average was used as one donor measurement. Multiple donors were tested and each data point on the column graphs represents the value for a donor. To test for differences in test conditions, test results were compared to the control condition of R848 alone. P-values were calculated using a mixed-effects one-way ANOVA, with corrections for multiple comparisons using a Dunnett's test.

Results—Treatment with 22:0 LYSO PC and R848 Hyperactivates Canine PBMCs

As described in Example 1, the combination of 22:0 LYSO PC (DAMP) and the TLR7/8 agonist R848 (PAMP) was found to have potent hyperstimulatory activity in human moDCs In order to assess whether this hyperstimulatory activity translates to other clinically relevant species, the ability of 22:0 LYSO PC+R848 to hyperactivate PBMCs isolated from canine whole blood was assessed. For each data set, PBMCs from multiple donors were used in lieu of moDCs due to the lack of canine-specific reagents available to induce bone fide canine moDCs. In brief, PBMCs were isolated from whole blood using density gradient centrifugation and then cultured for two days with the hyperactivating stimuli of interest.

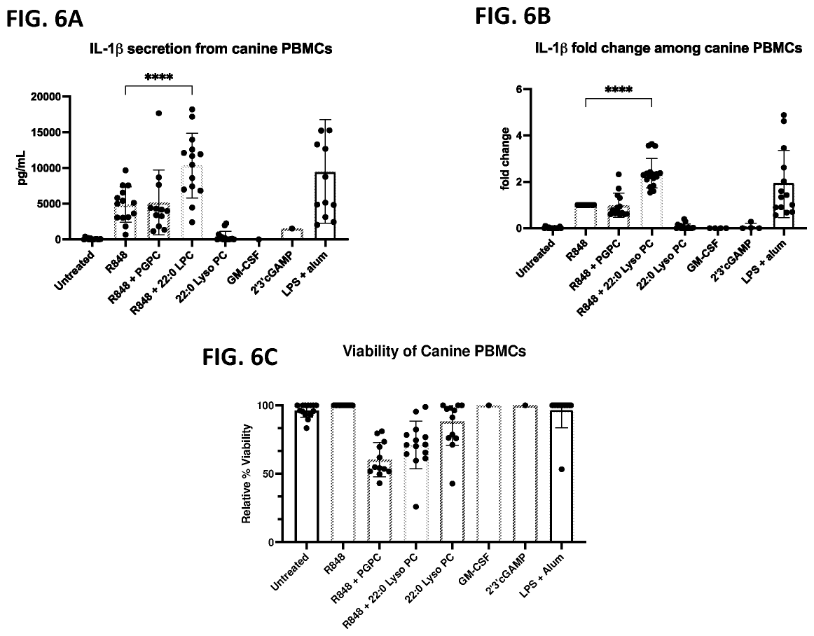

After two days in culture, hyperactivation was assessed by quantification of IL-1β in cell culture supernatants and measurement of cell viability. When treated with 22:0 LYSO PC and R848 together, canine PBMCs secreted comparable or higher levels of IL-1β compared to every other stimuli tested, both as concentration per mL, as well as fold change per donor relative to R848 alone (FIG. 6A-6B). Consistent with previous studies showing that monocytes, which make up 5-10% of PBMCs, can release IL-1β in response to activation with R848, canine PBMCs had elevated levels of IL-1β secretion with R848 alone compared to untreated cells. The pyroptotic combination of LPS+Alum elicited high levels of IL-1β as expected. Notably, while PGPC+R848 elicited similar levels of IL-1β compared to R848 alone, neither GM-CSF nor 2′3′cGAMP induced significant IL-1β secretion from canine PBMCs compared to untreated cells.

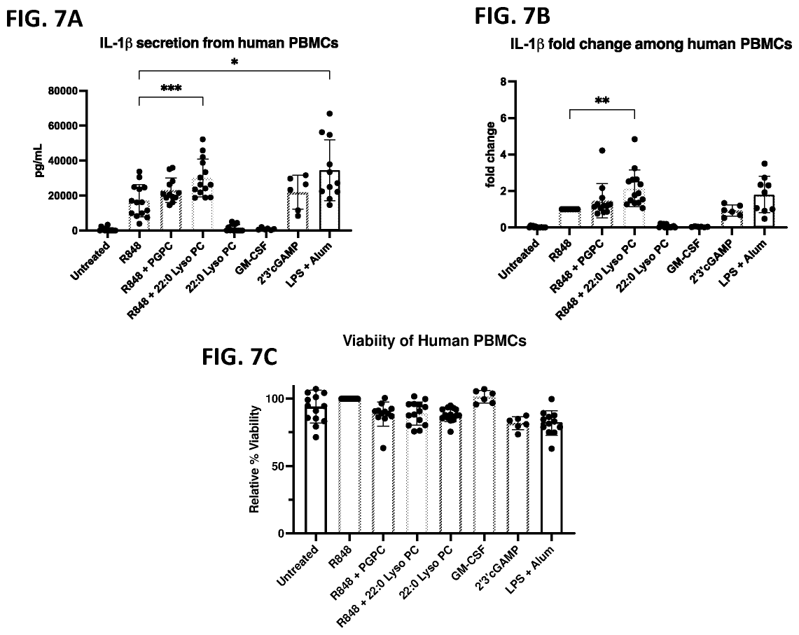

Although IL-1β can be detected one day after hyperactivation of canine PBMCs in cell culture supernatants, cell viability was evaluated two days post-hyperactivation to ensure enduring viability after IL-1β secretion. 22:0 LYSO+R848 did not significantly reduce relative cell viability (FIG. 6C). Interestingly, PGPC in combination with R848 proved to be somewhat toxic to canine PBMCs, although it was not observed to be toxic to human moDCs (Example 1) or human PBMCs (FIG. 7C). Together these data demonstrate that 22:0 LYSO+R848 elicits high levels of IL-1β secretion from canine PBMCs, which is indicative of hyperactivation.

Results—Treatment with 22:0 LYSO PC and R848 Hyperactivates Human PBMCs

The initial screens that identified 22:0 LYSO PC and R848 as having potent hyperactivation potential were performed with human moDCs as described in Example 1. To confirm that the observations made using canine PBMCs and human moDCs can be recapitulated in human PBMCs, hyperactivation experiments were also performed with PBMCs isolated from whole blood obtained from human donors. In brief, PBMCs were isolated from whole blood by density gradient centrifugation from multiple human donors and cultured for two days with the hyperactivating stimuli of interest.

Human PBMCs, like human moDCs and canine PBMCs, secreted IL-1β at levels higher or comparable to all other stimuli tested (FIG. 7A-7B). Similar to canine PBMCs, human PBMCs secreted IL-1β in response to R848 alone due to monocyte activation, and this was elevated by addition of 22:0 LYSO PC. The pyroptotic combination of LPS+Alum elicited high levels of IL-1β as expected. Consistent with observations in canine PBMCs, PGPC+R848 did not induce substantially higher levels of IL-1β than R848 alone. GM-CSF did not induce levels of IL-1β secretion from human PBMCs significantly above background levels produced by untreated cells.

Viability of human PBMCs was also assessed two days post-hyperactivation to ensure enduring viability of human PBMCs after IL-1β secretion. No significant decreases in human PBMC viability were observed after treatment with any of the stimuli (FIG. 7C). Together these data demonstrate that both human and canine PBMCs are hyperactivated by 22:0 LYSO PC+R848. Interestingly, canine PBMCs are hyperactivated to a greater extent by 22:0 LYSO PC+R848 than by PGPC+R848.

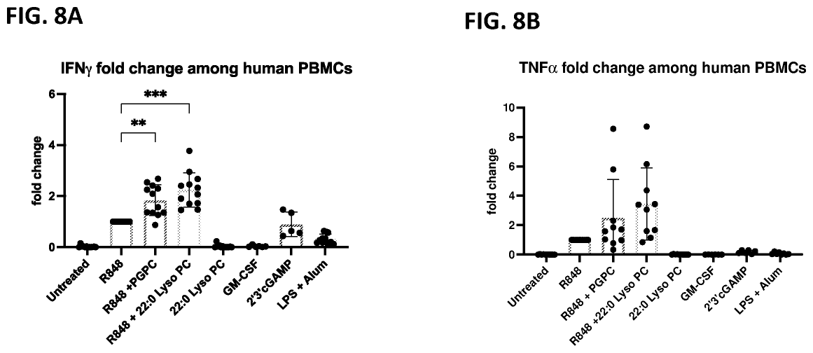

Because activated human PBMCs can secrete other cytokines in addition to IL-1β, the secretion of the pro-inflammatory cytokines IFNγ and TNFα in cell culture supernatants was measured two days post-hyperactivation. The combination of 22:0 LYSO PC+R848 induced the highest fold change per donor in both IFNγ secretion and TNFα secretion relative to R848 alone as compared to all other stimuli tested (FIG. 8A-8B). Notably, although LPS+Alum induced high levels of IL-1β secretion from human PBMCs (FIG. 7B), this combination of stimuli did not induce a fold increase in IFNγ or TNFα secretion. Moreover, neither GM-CSF nor 2′3′cGAMP elicited a substantial fold change in IFNγ secretion over R848 alone. These data indicate that the combination of 22:0 LYSO PC+R848 is superior at inducing secretion of the proinflammatory cytokines IFNγ and TNFα from human PBMCs.

### Example 3: 22:0 LYSO PC Hyperactivates Non-Human Primate Dendritic Cells and Peripheral Blood Mononuclear Cells

This example describes the hyperactivation of non-human primate moDCs and peripheral blood mononuclear cells (PBMCs) with a lipid DAMP in combination with a small molecule PAMP (R848).

### Materials and Methods

Non-human primate moDC differentiation and hyperactivation. Whole blood from rhesus macaques was diluted with an equal volume of PBS and over-layed on Ficoll-Paque PLUS solution. Cells were spun at 1000 rcf for 30 minutes at room temperature with no brakes. Buffy coat layer was collected, washed twice using PBS, and counted. To isolate monocytes from the PBMC, human CD14 microbeads from Miltenyi were used according to manufacturer's recommendations. Isolated monocytes were then differentiated into moDC over 6 days using recombinant human GM-CSF (50 ng/mL) and recombinant human IL-4 (25 ng/mL). Cells were grown in R10 media consisting of RPMI, 10% FBS, penicillin, streptomycin, L-glutamine, sodium pyruvate, and beta-mercaptoethanol.

Differentiated moDC were collected, counted, and plated at 1×10⁵ cells/well in a 96-well plate. Cells were treated with or without hyperactivating stimuli. Hyperactivation cultures were incubated for 2 days before assessing cell viability and IL-1beta secretion. Promega Cell Titer-Glo was used according to manufacturer's instructions to quantify ATP as a measure of cell viability. ELISA kits specific for non-human primate IL-1beta were purchased from Invitrogen, and assays were completed according to manufacturer's instructions.

Non-human primate PBMC hyperactivation. Whole blood from cynomolgus macaques was diluted with an equal volume of PBS. A solution of 90% Ficoll-Paque PLUS and 10% RPMI was made, and diluted whole blood was overlayed on top of the diluted Ficoll. Cells were centrifuged at 1000 rcf for 30 minutes at room temperature with no brakes. The buffy coat layer was collected and washed twice with PBS. If red blood cell contamination was present, cells were incubated in ACK lysis buffer for 2 minutes before being washed with PBS to remove lysed red blood cells.

Isolated PBMC were plated at 1×10⁶ cells/well and cultured with or without hyperactivating stimuli. After 2 days incubation, cells were assessed for viability, and cell culture supernatant was used to measure cytokine output. Commercially available kits such as Biolegend LegendPlex kits were used as directed by manufacturer's protocol to quantify cytokines of interest.

Statistical Analyses. Rhesus macaque moDC were generated from four animals. Each activation condition was tested in triplicate and reported data points are the mean of the triplicates for an animal donor sample. Note that hyperactivation using R848+41.3 uM PGPC was tested on only two samples due to limited cell numbers. Data were tested for statistical significance using ordinary one-way ANOVA.

PBMC were obtained from 13 cynomolgus macaques. Each activation condition was tested in triplicate, and the mean value of the triplicate is graphed for the animal sample. Repeated measures one-way ANOVA was used to test for statistical significance followed by Dunnett's multiple comparisons test.

### Results

If hyperactivation is an important biological function of the innate immune system, one would expect to find it evolutionarily conserved across multiple species. Additionally, further development of 22:0 LPC as a therapeutic would likely require testing in relevant animal models of disease. We hypothesized that closely related species such as non-human primates would also be responsive to 22:0 LPC-mediated hyperactivation.

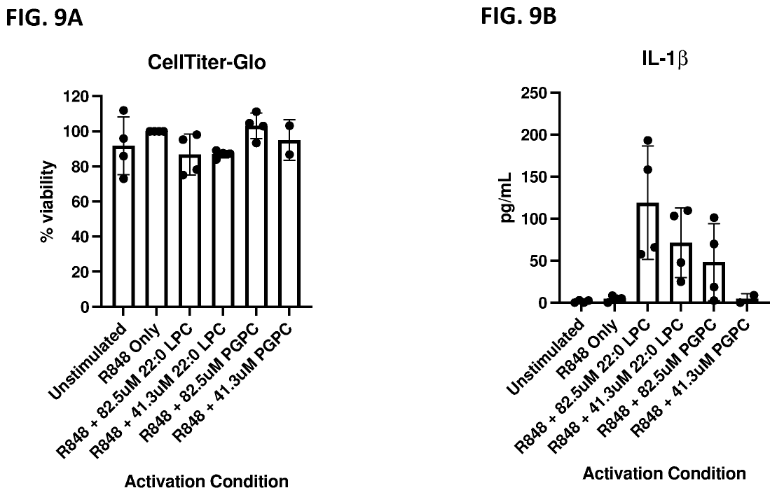

To test our hypothesis, we isolated monocytes from rhesus macaque whole blood. Using the same differentiation protocol as with human cells, the monocytes were differentiated into moDC using recombinant human GM-CSF and IL-4. Cells were plated and stimulated with or without R848 and with or without a hyperactivating lipid. After two days incubation, IL-1beta was detected in the cell culture supernatant. Interestingly, 22:0 LPC induced detectable IL-1beta at 82.5 uM and 41.3 uM whereas IL-1beta was only detectable at 82.5 uM using PGPC (FIG. 9B). These results agree with data generated using human moDC, demonstrating 22:0 Lyso PC to be more potent than PGPC. Additionally, cells remained viable across the conditions tested (FIG. 9A).

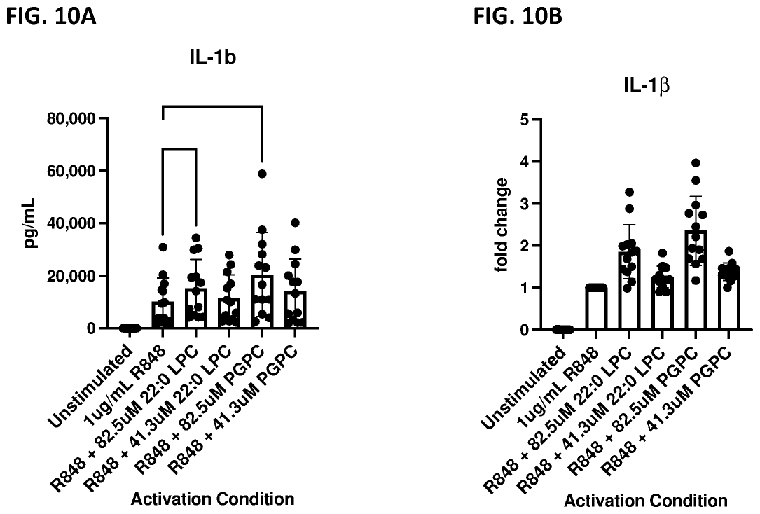

Studies using human and canine PBMC demonstrated that cells within the population respond to hyperactivation. Since non-human primate moDC can be hyperactivated, we sought to test whether NHP-derived PBMC can also be hyperactivated. Whole blood derived from cynomolgus macaques was processed to isolate PBMC. Cells were plated at 1×10⁶ cells/well and hyperactivated with 22:0 Lyso PC or PGPC at 82.5 and 41.3 uM concentrations. As negative controls for hyperactivation, PBMC were left untreated or only stimulated with 1 μg/mL R848. Cell viability was maintained in all tested conditions. Similar to canine and human PBMC, R848 stimulation induced IL-1beta secretion. Adding 82.5 uM of either 22:0 Lyso PC or PGPC to induce hyperactivation increased the IL-1beta output (FIG. 10A). The effect was particularly evident when quantifying the fold change in IL-1beta output of hyperactivating conditions compared to R848 stimulation alone (FIG. 10B).

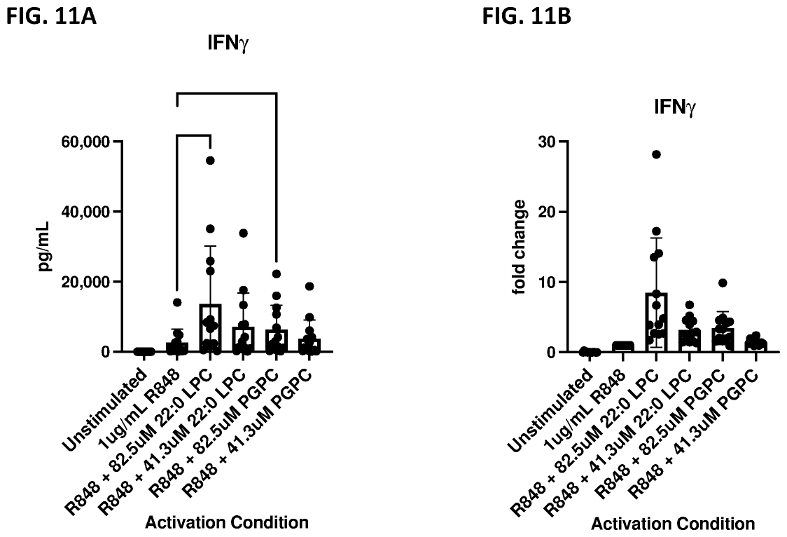

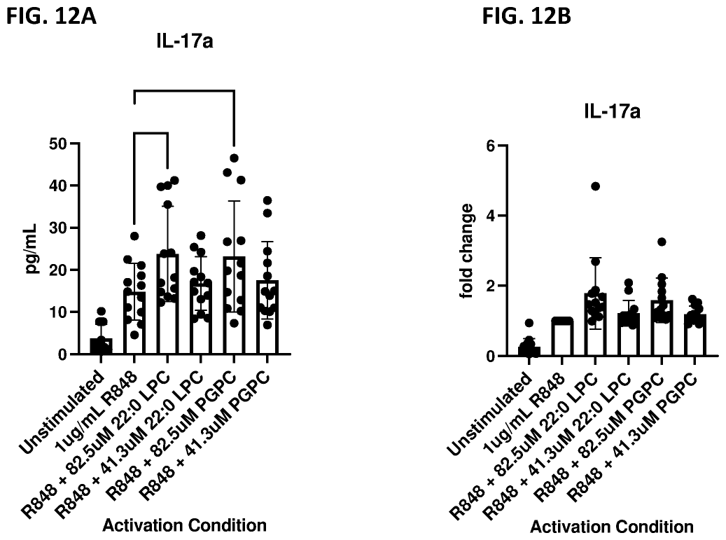

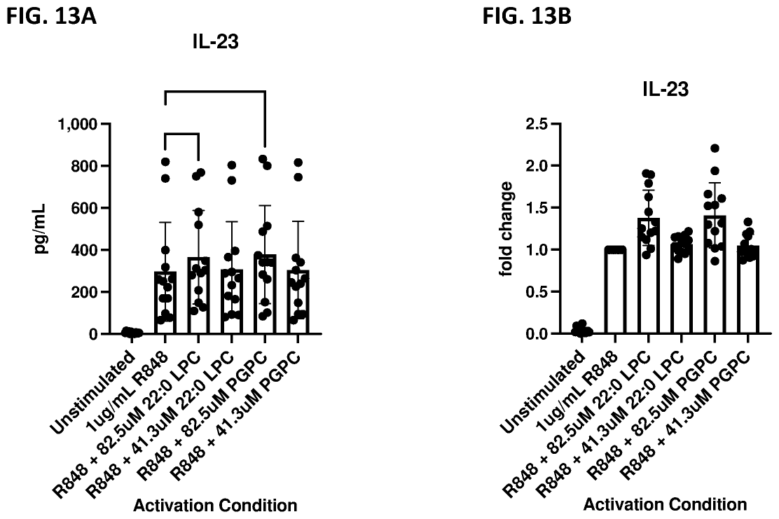

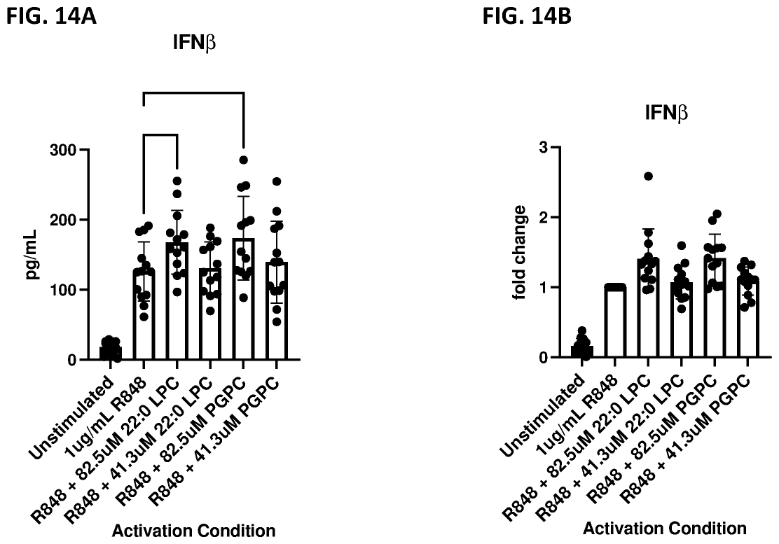

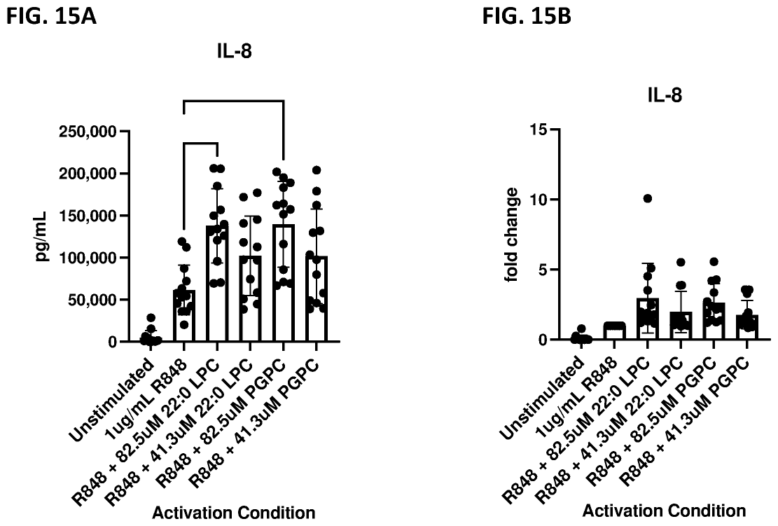

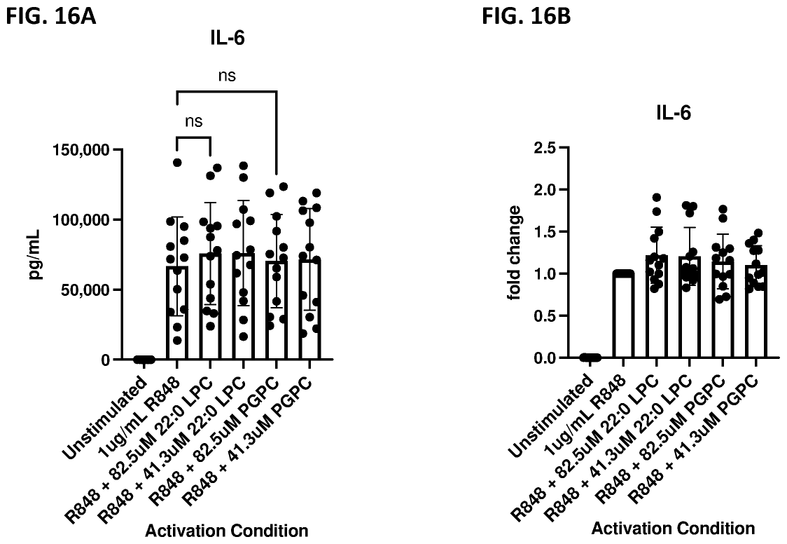

Similar to human PBMC, cynomolgus macaque PBMC had significantly increased IFN-gamma output when hyperactivated with 82.5 uM 22:0 Lyso PC or PGPC for two days. To extend these observations and identify other cytokines possibly increased by hyperactivation, a multiplexed bead-based assay was used to quantify inflammatory cytokines. Biolegend's LegendPlex kits function similarly to an ELISA, but the primary antibodies are bound to beads instead of a plate surface. Distinct bead populations allow for multiple cytokines to be quantified simultaneously within one reaction. Using the bead assay, multiple cytokines were identified to increase with hyperactivation. IFN-gamma, IL-17a, IL-23, IFN-beta, and IL-8 were increased when PBMC were hyperactivated using 82.5 uM 22:0 Lyso PC or PGPC compared to treatment with R848 alone (FIG. 11A-15A). These increases were particularly evident when fold changes were calculated for each animal sample to compare between hyperactivation and R848 activation (FIG. 11B-15B). The increases in cytokine levels were dose dependent upon the amount of lipid used. Fold changes were not as markedly increased when using 22:0 Lyso PC or PGPC at 41.3 uM, and at this concentration, increases in cytokine secretion were not always statistically significant. Interestingly, not all cytokines were affected by hyperactivation. For example, IL-6 secretion remained relatively unchanged by addition of a hyperactivating lipid (FIG. 16A). The data graphed as fold change looks particularly distinct from trends observed with hyperactivation-dependent cytokines (FIG. 16B).

Altogether, these data indicate that 22:0 Lyso PC is a hyperactivating lipid in both rhesus macaques and cynomolgus macaques. Furthermore, non-human primate cells can be hyperactivated, similar to what has been observed in other species. These data are particularly intriguing because cytokines such as IFN-gamma and IL-17a are unlikely to be produced by phagocytic cells, which we believe are the main responders to the hyperactivating stimuli used. If true, then hyperactivated cells are likely inducing secondary effects on neighboring cells such as T cells which can express IFN-gamma and IL-17a.

### Example 4: Hyperactivated Dendritic Cells Stimulate Potent CD4+Th1 Cell Responses

This example describes the effects of hyperactivated human moDCs on human memory and naïve CD4+ T cells.

### Materials and Methods

Human moDC coculture with memory CD4 T cells. Human monocytes were isolated from leukopheresis products using Miltenyi StraightFrom LeukoPak CD14 microbeads as directed by manufacturer. Custom PBMC isolation microbead kits from Miltenyi were used to separate PBMCs from red blood cells and granulocytes from the same blood products. After PBMC purification, Miltenyi human memory CD4+ T cell isolation kits were used to negatively select the CD45RO+ fraction. Cells were cryogenically stored until ready for use in experiments.

Human monocytes were differentiated into dendritic cells as previously described using recombinant human GM-CSF and IL-4 for 6 days. moDCs were plated in R10 media at 5E4 cells/well into 96-well U-bottom microplates for hyperactivations. R848 and lipid were added onto cell cultures, and hyperactivations were incubated overnight. T cell stimulating anti-CD3 antibody was added into the culture (with or without blocking anti-IL-1beta antibody) prior to T cell addition. Autologous frozen memory CD4 T cells were thawed, resuspended in R10 media and added at 2.5×10⁵ cells/well (ratio 1:5, DC:T cell). After two days incubation, cell culture supernatant was collected and used for measuring cytokine secretion. Commercially available kits such as Biolegend LegendPlex kits were used as directed by manufacturer's protocol to quantify cytokines of interest.

Four donors were tested with each experimental condition tested in triplicate. Each data point represents the mean value of a donor triplicate. Data were tested for statistical significance using two-way ANOVA followed by Tukey's multiple comparisons tests.

Mixed lymphocyte reaction. Human monocytes were isolated from leukopheresis products using Miltenyi StraightFrom LeukoPak CD14 microbeads as directed by manufacturer. To isolate naïve CD4 T cells, whole blood was diluted with an equal volume of PBS. Diluted blood was layered on top of Ficoll-Paque PLUS solution and spun at 1000 rcf for 20 minutes at room temperature with no brakes. Buffy coat layer was collected, washed, and counted. Miltenyi human naïve CD4 T cell microbead isolation kits were used to isolate cells of interest from the PBMC according to manufacturer's instructions. Collected cells were cryogenically stored in 90% FBS and 10% DMSO.

Monocytes were differentiated into moDC. moDC were counted and plated in U-bottom plates at 2×10⁴ cells/well and treated with hyperactivating stimuli or control conditions. After one day of culture, 1×10⁵ cells/well naïve CD4 T cells from a different (allogeneic) human donor were thawed and added to the cultures with 100 ng/mL IL-2. Cells were cocultured for 5 days with an IL-2 feed on the third day of coculture. After 5 days, cells were reactivated for four hours using Leukocyte Activation Cocktail with BD GolgiPlug (BD Biosciences). Cells were stained to determine the resulting polarization of activated T cells via flow cytometry. Intracellular staining targets required the use of BD Biosciences Transcription Factor Buffer set to fix and permeabilize cells prior to the intracellular staining step.

Ten moDC donor samples were tested in MLR assay. Staining results were analyzed using FlowJo software, and consistent gating strategies were applied to all samples. Frequency of activated cells was graphed as a percentage of CD4 T cells in each sample. Paired t-tests were used to determine statistical significance.

### Results—Hyperactive Dendritic Cells Strongly Reactivate Th1 Memory CD4 T Cells

One key characteristic of hyperactive dendritic cells is that they secrete mature IL-1beta while remaining viable. In contrast, non-hyperactive cells undergo pyroptosis when secreting IL-1b. These two characteristics are of particular interest because continued cell viability allows dendritic cells the opportunity to interact with other cells such as T cells. These cell-cell interactions provide an opportunity for DC to present antigens and provide co-stimulatory and inflammatory signals to T cells. As discussed above in other examples, hyperactivation of PBMC could potentially activate other cell types. Interestingly, DC have been reported to more potently reactivate memory CD4 T cells when compared to non-cell based methods such as using plate-bound anti-CD3 and anti-CD28 agonist antibodies (Jain et al., Nat Commun, 9(1):3185, 2018). The mechanism of enhanced T cell reactivation in the published study depended on IL-1beta signaling from the DC to the T cells. However, the effect of different DC activation states on T cell responses was not explored. Given that hyperactivated DC robustly produce IL-1beta and their viability allow for continued cell-cell interactions, we hypothesized that hyperactivation would be the most potent cell state of dendritic cells to reactivate memory CD4 T cells.

To test our hypothesis, human monocytes were differentiated into moDC. The moDC were either left unstimulated, or activated with R848, or hyperactivated with R848 and 22:0 LPC, or sham treated with a non-hyperactivating lipid (R848 and 10:0 LPC). After a day of incubation, autologous T cells were added to the cultures. To simulate peptide-MHC recognition, TCR-stimulating anti-CD3 antibody was added at extremely limiting concentrations (300× less than what was used by Jain et al., supra, 2018, which was already noted to be a very dilute concentration). To test whether T cell responses were mediated by IL-1b, additional experimental conditions were set up where anti-IL-1beta blocking antibody was added to cultures to inhibit IL-1beta signaling. As additional controls, some moDC stimulations did not receive T cell additions to ensure that cytokine responses measured did not come directly from the moDC. Stimuli were also plated to empty wells and then T cells were later added to confirm that the stimuli did not directly affect T cells without the need of moDC.

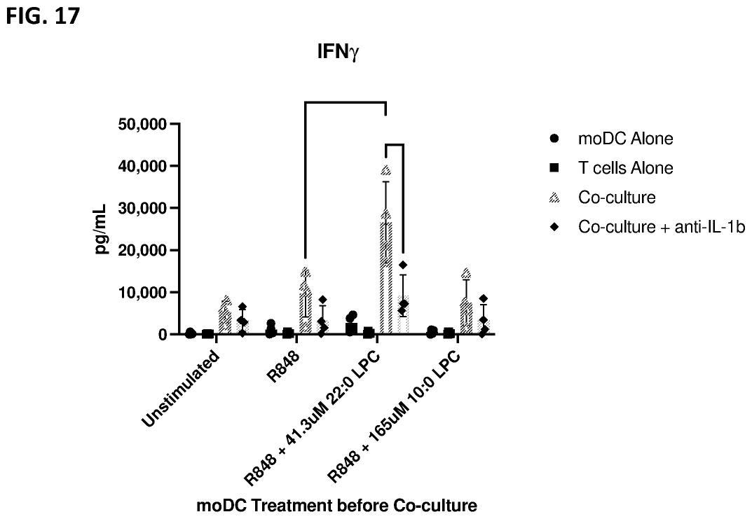

T cell response cytokines were measured from culture supernatants two days after addition of the T cells. IFN-gamma was minimally detected when moDC or T cells were cultured alone. When cocultured, IFN-gamma was produced, indicating a Th1 response from T cells. Given the limited TCR stimulation via anti-CD3 antibody, R848-treated moDC did not produce a significantly stronger Th1 response compared to unstimulated moDC. However, when R848 was combined with the hyperactivating lipid 22:0 LPC, a significantly stronger Th1 response was produced (FIG. 17). These elevated IFN-gamma levels were not detected when using a non-hyperactivating lipid such as 10:0 LPC. Furthermore, the hyperactivated co-cultures were significantly reduced in IFN-gamma production when anti-IL-1beta was added to the co-culture, suggesting that the enhanced Th1 response depended on IL-1beta signaling.

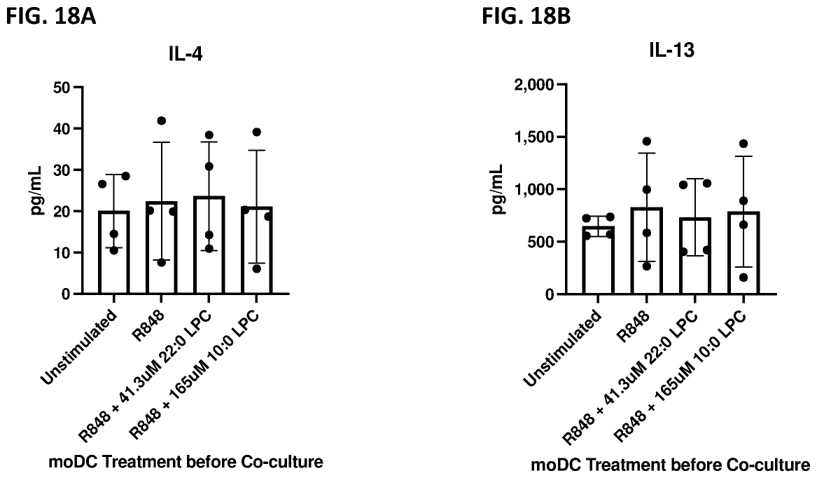

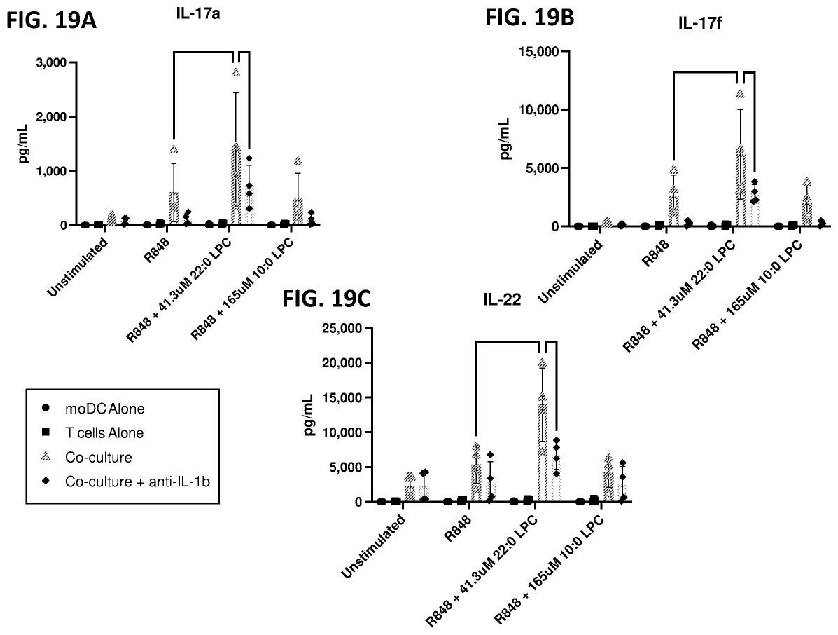

In contrast, Th2 cytokines IL-4 and IL-13 were not enhanced by moDC hyperactivation. moDC treated with the various stimuli resulted in similar IL-4 and IL-13 responses from co-cultured T cells that were not statistically significant (FIG. 18A-18B). Th17 cytokines IL-17a, IL-17f, and IL-22 were also measured. Th17 cytokines followed similar trends observed with IFN-gamma (FIG. 19A-C). Hyperactivated moDC induced statistically significant increases in Th17 cytokine production whereas non-hyperactivated moDC did not. Enhanced Th17 cytokine production depended on IL-1beta signaling because anti-IL-1beta antibodies significantly reduced cytokine levels. These results suggested that hyperactivated moDC preferentially potentiate Th1 and Th17 responses whereas the Th2 response remains unaffected. In the context of anti-tumor responses, hyperactivated DC are contemplated to be better suited to reactivate tumor-responsive memory CD4 T cells because they can provide IL-1beta in the proper context during antigen presentation. Given the low anti-CD3 stimulation used in our experimental system, hyperactive DC are contemplated to be better equipped to stimulate reactivation despite weak TCR interactions with peptide-MHC, such as those found in cancer.

Results—Hyperactive moDCs Polarize Naïve CD4 T cells to Th1 Responses

Hyperactivation improves moDC ability to reactivate Th1 and Th17 memory CD4 T cell responses. In some disease contexts, CD4 T cells may not have been previously activated and would therefore be naïve. To investigate the effects of hyperactivated moDC on naïve CD4 T cells, a widely used immunological assay was employed: the mixed lymphocyte reaction (MLR). moDC were either activated with R848 or hyperactivated with R848 and 22:0 LPC. After one day incubation, naïve CD4 T cells from an allogeneic donor were added to the culture. The mismatch between TCR and peptide-MHC provides TCR stimulation for a fraction of T cells. IL-2 is added to provide a basal T cell growth signal. Cells were cocultured for 5 days before staining and flow cytometry analysis. Flow cytometry was chosen because the technique gathers data with specificity at the single cell level. To determine CD4 T cell polarization, activated cells were stained for effector cytokines and associated polarization transcription factors.

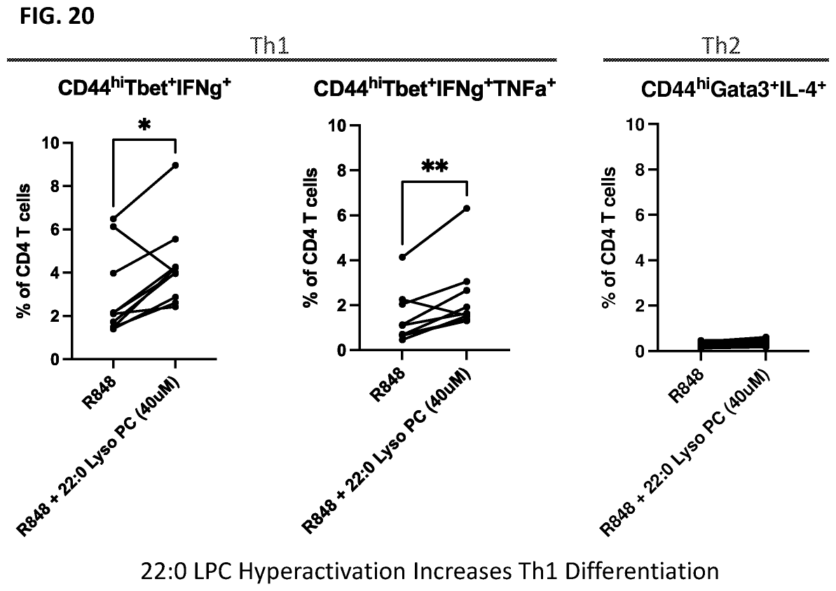

To analyze Th1 polarization, cells were stained for the transcription factor Thet and cytokines IFN-gamma and TNF-alpha. Compared to R848 activation, hyperactivation using 22:0 LPC increased the frequency of Th1 cells (FIG. 20). Moreover, polyfunctional Th1 polarized cells (as noted by their dual expression of IFN-gamma and TNF-alpha) were increased in frequency when cultured with hyperactivated moDC (FIG. 20). Th2 polarization was studied by staining for Gata3 and IL-4. In comparison to the Th1 response, minimal Th2 polarization occurred (FIG. 20). Interestingly cells were also stained for RORg and IL-17, but these cells were not detected. The lack of Th17 cell differentiation from naïve CD4 T cells contrasts with the results observed when memory CD4 T cells were cocultured with moDC. Altogether the results of these studies indicate that hyperactivation using 22:0 LPC enhances the polarization of naïve CD4 T cells to a Th1 response upon activation.

### Example 5: Formulation of 22:0 LYSO PC into Micelles

This example describes the preparation and testing of surfactant-containing formulations of 22:0 Lyso PC.

### Materials and Methods

Formulation of 22:0 Lyso PC with poloxamers. The following surfactants were dissolved in water at 1%, 2%, or 5.5%: Poloxamer 407 (KP407), Poloxamer 188 (KP188), Cremophor EL (K EL), Cremophor RH40 (K RH), Pluronic P84 (P-84), and Pluronic P123 (P-123). 22:0 Lyso PC was resuspended in the surfactant solution and agitated at 4° C. for an hour. Solutions were then brought to room temperature to form micelles. Using 10× concentrated PBS, salts were added to the solution to stabilize molecular interactions and provide physiologically relevant osmolarity. Lipid solutions were either left unfiltered or filtered through a pore size of 0.45 um to remove insoluble lipid flakes. Lipids were further diluted and added onto human moDC cell cultures along with 1 ug/mL R848. Targeted lipid concentrations were based on the scenario where the lipid is fully bio-available prior to filtration. However, filtration of insoluble lipid reduces the bio-available supply of lipid. Thus, IL-1beta as a measure of hyperactivity can be used as an indicator of 22:0 Lyso PC incorporation into micelles and thus into solution. One day after hyperactivation, cell culture supernatants are collected to measure cytokine secretion, and cells are used to measure viability via Cell Titer-Glo.

The solvent evaporation methodology utilizes methanol or ethanol to completely dissolve the 22:0 Lyso PC before mixing with KP407. The dissolved lipid solution is mixed with 5.5% KP407 under stirring for 90 minutes at room temperature to evaporate the alcohol solvent and induce particle formation with the poloxamer and lipid. Salts are added to the solution to stabilize the molecular interactions and bring the solution to a physiologically relevant osmolarity. The solutions are then either left unfiltered or filtered through a pore size of 0.45 um to remove insoluble lipid flakes or particle aggregates. Lipids are then added to human moDC cultures with 1 ug/mL R848. One day after hyperactivation, cell culture supernatants were collected to measure cytokine secretion, and cells were used to measure cell viability.

Particle size was quantified by Dynamic Light Scattering (DLS). Particle size was assessed for particles in solution after 0.45 um filtration of micelles made using resuspension in KP407 or solvent evaporation particle synthesis.

Data from these studies in poloxamer formulation are shown from one human moDC donor sample. Individual data points represent the mean of triplicates.

Results—Incorporation of 22:0 LYSO PC into Micelles

22:0 Lyso PC is a lipid that is mostly insoluble in aqueous solution. Our initial method of adding 22:0 Lyso PC to cell cultures was to simply resuspend the lipid in culture media. However, lipid material was clearly visible as flakes in solution. To administer a consistent dose, having the lipid solubilized would be ideal. This would be most important in animal studies and also as a developed therapy for human use. Given that the hyperactivating molecule is a lipid, we hypothesized that 22:0 Lyso PC could be incorporated into micelles.

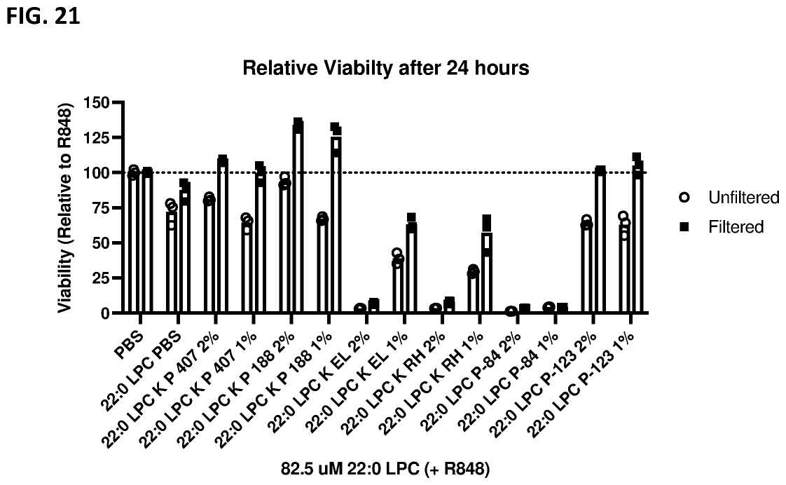

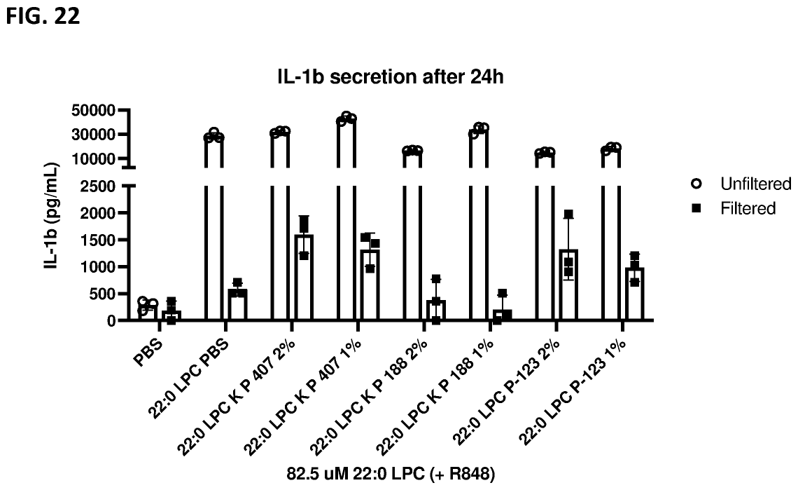

To test our hypothesis, we screened a set of surfactants which can aid in the micellization of 22:0 Lyso PC. Lyophilized lipids were mixed with water solutions containing 1 or 2% surfactant. The solutions were refrigerated during the mixing to allow the surfactants to monomerize and interact with the lipid. The solutions were then warmed to room temperature to allow micellization. Using 10× concentrated PBS, salts were added to the solution to help stabilize molecular interactions and bring the solutions to a physiologically relevant osmolarity. The 22:0 Lyso PC micelles were then left unfiltered or passed through 0.45 um filters to remove insoluble lipid. These lipid stocks were then further diluted and added to human moDC cultures to hyperactivate cells. Some surfactants such as K EL, K RH, and P-84 caused loss of cell viability and were not ideal to use (FIG. 21). In comparison, KP407, KP188, and P-123 did not compromise cell viability. We then quantified the IL-1beta secretion from these various stimulation conditions where cell viability was maintained. When the lipid was left unfiltered, high IL-1beta secretion was observed, similar to our standard method where 22:0 Lyso PC is resuspended in PBS and added to cells (FIG. 22). Filtration of the lipid significantly decreased the IL-1beta secreted by moDC, suggesting that insoluble lipid also contributes to hyperactivation (FIG. 22). When 22:0 Lyso PC was formulated with KP407 or P-123 and filtered, IL-1beta secretion was improved compared to filtered 22:0 Lyso PC in PBS (FIG. 22). These data demonstrate that 22:0 Lyso PC solubilized as micelles maintain their ability to induce DC hyperactivation, and improved solubility increases DC hyperactivation. This initial study demonstrated that a consistent solution of 22:0 Lyso PC can be achieved by using poloxamers and pluronics to incorporate the lipid into micelles.

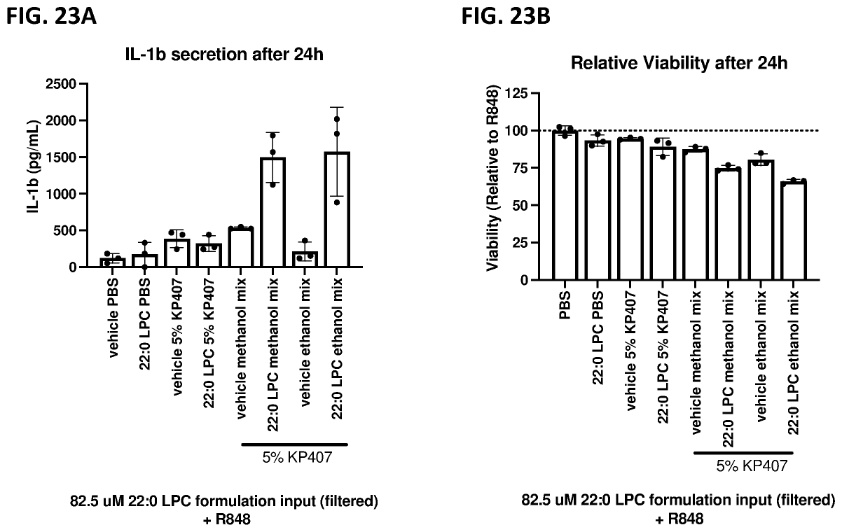

KP407 was selected as a poloxamer for further study and optimization. Since 22:0 Lyso PC is mostly insoluble in aqueous solution, we hypothesized that fully solubilized lipid would more readily associate with KP407. 22:0 Lyso PC is soluble in ethanol or methanol, and both these alcohols are miscible with water. To optimize the incorporation of 22:0 Lyso PC into particles, the lipid was first dissolved in methanol or ethanol. The dissolved lipid solution was mixed with 5.5% KP407 under stirring, and allowed to stir at 150 rpm for 90 minutes at room temperature to evaporate the alcohol solvent and induce particle formation with the poloxamer and lipid. Water was added to return the KP407 concentration to 5.5% after evaporation. Using 10× concentrated PBS, salts were added to stabilize particle formation and reach a physiologically relevant osmolarity, bringing the final concentration of KP407 to 5% in PBS. After filtering through a pore size of 0.45 um, lipid solutions were diluted and used to hyperactivate cells at a theoretical target concentration of 82.5 uM. 22:0 Lyso PC not incorporated into KP407 micelles induced minimal amounts of IL-1beta because the majority of the material was filtered out (FIG. 23A). However, mixing KP407 increased IL-1beta secretion (FIG. 23A). By using methanol or ethanol to first solubilize the lipid, IL-1beta secretion further increased, suggesting that more lipid was incorporated into particles using this solvent evaporation strategy. The majority of the cells remained viable for each condition, but the solvent evaporation method did reduce cell viability by approximately 25% (FIG. 23B). Interestingly, the viability of the samples treated using the vehicle solvent evaporation method did not experience as much of a drop in viability, suggesting that increasing the bioavailability of the 22:0 Lyso PC might allow for a lower dose of 22:0 Lyso PC to be used.

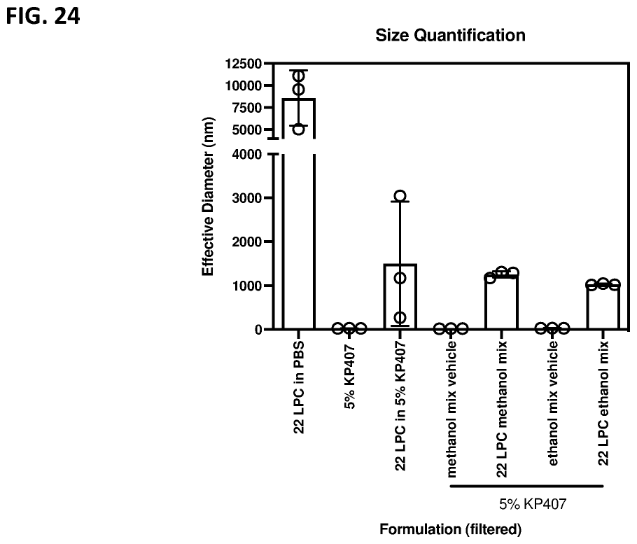

To characterize the particle size, dynamic light scattering was employed. Readings performed on 22:0 Lyso PC in PBS and 22:0 Lyso PC in 5% KP407 resulted in poor quality sizing data with wide variability in sample readings due to inconsistent particle size. In contrast, particles created using solvent evaporation were consistently approximately 1500 nm in diameter (FIG. 24), while preparations of micelles lacking 22:0 Lyso PC lacked particles of this size. Thus, the interaction between 22:0 Lyso PC and KP407 allowed for the formation of stable particles of about 1500 nm in diameter. These particles are not solid, and given the measured size of the particles, can likely deform, allowing them to pass through the smaller filter pores.

### Example 6: Hyperactivation of Murine Dendritic Cells In Vitro and In Vivo

This example describes the testing of KP407-containing formulations of 22:0 Lyso PC and/or R848 on murine cells in vitro and in a murine tumor model in vivo.

### Materials and Methods

Murine FLT3L-differentiated, bone marrow-derived DC (BMDC) generation. Leg femur and tibia were removed from mice, cut with scissors and flushed into sterile tubes. Bone marrow suspension was treated with ACK for 1 minute, then passed through a 40 um cell strainer. Cells were counted and resuspended in media consisting of complete IMDM containing 10% FBS, penicillin and streptomycin, and supplements of L-glutamine and sodium pyruvate (I10). Cells were then plated at 5E6 bone marrow cells per well in a P12 plate. Recombinant mouse FLT3L (Miltenyi) was added to cultures at 200 ng/mL. Differentiated cells were used for subsequent assays on day 9. The efficiency of differentiation was monitored by flow cytometry using BD Symphony A3, and CD11c+MHC-II+ cells were routinely above 80% of living cells. For each experiment, five mice were used to collect BM and to generate DCs.

Murine FLT3L-differentiated BMDC (FLT3L-BMDC) hyperactivation. BMDCs were washed with PBS and re-plated in FLT3L-containing 110 at a concentration of 1.5×10⁵ cells/well. Stimuli were added to cultures at indicated concentrations for a final volume of 200 uL/well. Twenty-four hours post-stimulation, cell culture supernatant was collected after centrifugation and stored at −20° C. to measure cytokine secretion. IL-1b, IL-6, IL-12p40, and TNF-alpha ELISA were performed using eBioscience Ready-SET-Go! (now ThermoFisher) ELISA kits according to the manufacturer's protocols. Cell viability was measured using Promega's Cell Titer-Glo reagent kits.

Flow cytometry. After FcR blockade, treated FLT3L-BMDCs were washed and stained in PBS with Live Dead Fixable dye (ThermoFisher) for 20 minutes at 4° C. Cells were then washed again and stained for 20 minutes at 4° C. in MACS buffer (PBS with 1% FCS and 2 mM EDTA) containing the following fluorescently conjugated antibodies purchased from BioLegend: anti-CD11c, anti-I-A/I-E, anti-H-2Kb, anti-SIRPa, anti-CD24, anti-CD40, anti-CD45R, anti-CXCL16 and anti-CCR7. Data were acquired on a BD FACS Symphony (Becton-Dickenson). Data were analyzed using FlowJo software (Tree Star). Experimental conditions were tested in triplicate, and conditions were tested in two or three independent experiments.

DC infiltration in the skin dLN. To assess the infiltration of DCs in the skin draining lymph node (dLN), mice were subcutaneously injected with R848 in combination with 22:0 Lyso PC that was resuspended in KP407 at 10% or 15% or 20%. 24 hours post-injection, the skin draining lymph nodes (dLN) were dissected. A single cell suspension was prepared, and cells were stained in PBS with Live Dead Fixable dye (ThermoFisher) for 20 minutes at 4° C. Cells were then washed again and stained for 20 minutes at 4° C. in MACS buffer (PBS with 1% FCS and 2 mM EDTA) containing the following fluorescently conjugated antibodies: anti-CD 11c and anti-I-A/I-E (MHC-II). To determine the absolute number of CD11c+MHC-Ilhigh among living cells, countBright counting beads (ThermoFisher) were used, following the manufacturer's protocol. Data were acquired on a BD FACS Symphony (Becton-Dickenson). Data were analyzed using FlowJo software (Tree Star). Five mice were used for each experimental group.

Antigen uptake and presentation assay. To examine antigen uptake and the endocytic ability of BMDCs, pHrodo™ Red Dextran 10,000 MW was used (ThermoFisher). Briefly, FLT3L-derived BMDCs previously cultured with media alone, or treated for 24 hours with R848 alone or in combination with 22:0 Lyso PC, were incubated with pHrodo™ Red Dextran (40 ug/ml) for 45 minutes at 37° C., or at 4° C. (as a control for surface binding of the antigen). BMDCs were then washed and stained with Live/Dead Fixable Violet dye (ThermoFisher) to distinguish living cells from dead cells. Cells were then fixed with BD fixation solution and resuspended in MACS buffer. Red (APC) fluorescence of live cells was measured by flow cytometry. Fluorescence values of BMDCs incubated at 37° C. were reported as Mean Fluorescence Intensity (MFI) of pHrodo™ Red Dextran associated cells as normalized to MFI of pHrodo™ Red Dextran associated cells incubated at 4° C. To measure the efficiency of OVA peptide presentation on MHC-I, FLT3L-BMDCs were treated as described above and incubated with Endofit-OVA protein (0.5 mg/ml) for 1 hour at 37° C. Cells were then washed with MACS buffer and stained at 4° C. for 20 to 30 minutes with APC-conjugated anti-mouse H-2Kb antibody (BioLegend), and a PE-conjugated antibody that binds to H-2Kb bound to the OVA peptide “SIINFEKL” (BioLegend). Non-OVA-treated DCs served as a negative control and isotype controls were used as a staining control. The percentage of cells associated with the OVA peptide on MHC-I was calculated by flow cytometry. Data were acquired on a Symphony A3 flow cytometer (Becton-Dickenson) and analyzed with FlowJo software (Becton-Dickenson). Experimental conditions were tested in triplicate.

Ex vivo whole tumor lysate preparation. Syngeneic whole tumor lysates (WTL) were prepared from tumors explants of unimmunized tumor-bearing mice. Briefly, tumors from unimmunized mice bearing a tumor 10-12 mm of size were mechanically disaggregated using gentle MACS dissociator (Miltenyi Biotec) and enzymatically digested using the tumor Dissociation Kit (Miltenyi Biotec) following the manufacturer's protocol. After digestion, tumor cell suspensions were washed with PBS and passed through 70 um and then 30 um filters. Single cell suspension was depleted of CD45+ cells using anti-CD45 TILs microbeads (Miltenyi Biotec). Tumor cells were then counted and resuspended at 1×10⁷ cells/ml then lysed by 3-4 cycles of freeze-thawing. The lysed cells were further disrupted by repeatedly passing the material through an 18G, then 21G, and finally 25G needles. Lysate was filtered again through 70 um and 30 um cell strainers and stored in aliquots at −80° C. until use. WTL were used for immunotherapy at a concentration equivalent to 5.75E5 tumor cells per mouse.

Immunotherapeutic immunization and tumor challenges. For immunizations in the context of an immunotherapeutic approach, C57BL/6J were injected on the left flank with 5×10⁴ B16F10 cells. When tumors were palpable, mice were either left unimmunized, immunized with WTL alone, immunized with WTL in combination with R848, or immunized with WTL in combination with R848 and 22:0 LPC prepared in PBS or KP407. Mice received two boost injections at 7-day intervals.

Quantification and statistical analysis. In in vivo studies, n refers to the number of animals per condition from one or two independent experiments. Statistical differences were calculated by using unpaired two-tailed Student's t test, or one-way ANOVA with Tukey post-test. Dependent samples were analyzed with paired t tests. Statistical significance for experiments with more than two groups was tested with two-way ANOVA with Tukey multiple comparison test correction. All experiments were analyzed using Prism 7 (GraphPad Software). Graphical data was shown as mean values with error bars indicating the SD or SEM. P values of <0.05 (*), <0.01 (**) or <0.001(***);%0.0001 (****) indicated significant differences between groups.

### Results

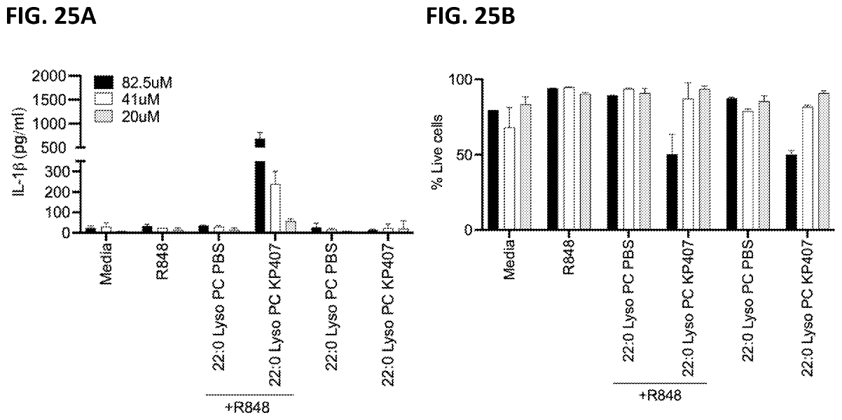

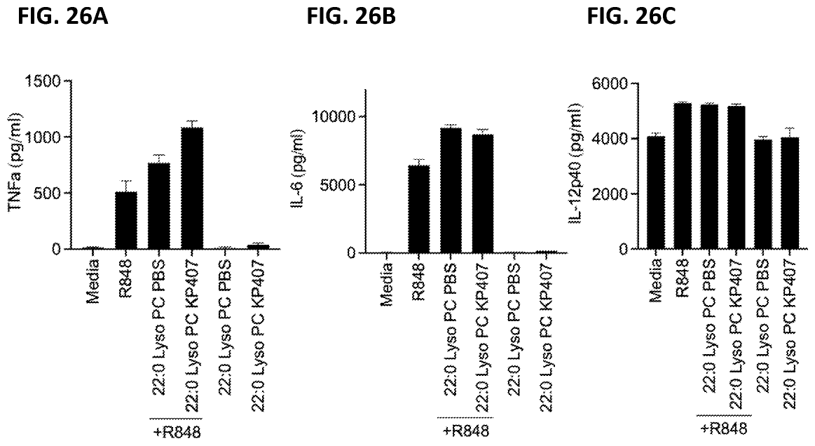

Mice are a critical experimental model, particularly for testing cancer therapies. To assess the therapeutic effect of 22:0 Lyso PC in murine tumor-bearing models, we first sought to determine if 22:0 LPC hyperactivate murine dendric cells. Dendritic cells were differentiated from the mouse bone marrow using murine FLT3L recombinant protein. To hyperactivate murine DCs, 22:0 Lyso PC was prepared using two methods. 22:0 Lyso PC was resuspended in PBS to be added onto cells, or the lipid was resuspended in 5% Kolliphor P407 (KP407). When combined with R848 (1 ug/mL), different concentrations of 22:0 Lyso PC resuspended in PBS were unable to induce IL-1beta secretion. In contrast, 22:0 Lyso PC formulated in KP407 induced IL-1beta secretion in a dose dependent manner, demonstrating that mouse cells can be hyperactivated by 22:0 Lyso PC (FIG. 25A). As expected, untreated DCs or DCs treated with R848 alone or 22:0 Lyso PC without R848 failed to induce IL-1beta secretion. Of the lipid concentrations tested, 41 uM 22:0 Lyso PC formulated in KP407 was superior in supporting IL-1beta secretion while retaining cell viability (FIG. 25B). These data suggest that incorporation of 22:0 Lyso PC into micelles makes the lipid bioavailable to murine dendritic cells. Hyperactive DCs acquire the ability to release IL-1beta while remaining viable, but they should also share similar properties as active DCs treated with PAMPs alone. To test if hyperactive DCs treated with R848 and 22:0 Lyso PC retained their classical DC functions (pro-inflammatory cytokine secretion, antigen uptake, antigen presentation, co-stimulatory molecule expression and chemokine receptor), DCs were treated as above with media or R848 alone, or DCs were treated with R848 in combination with 41 uM 22:0 Lyso PC for 24 hours. As expected, R848 stimulation induced high levels of pro-inflammatory cytokine secretion such as TNF-alpha secretion or IL-6 as compared to naïve DCs that were treated with media alone. Similarly, hyperactive DCs treated with R848 in combination with 22:0 LPC resupended in PBS or KP407 induced high TNF-alpha and IL-6 secretion, with a small boost in TNF-alpha secretion following DC stimulation with R848 and 22:0 Lyso PC in KP407 (FIG. 26A-26B). Notably, IL-12p40 cytokine, a key driver of Th1 response was induced by R848 stimulation in the presence or absence of 22:0 LPC (FIG. 26C). Overall, these data highlight that 22:0 Lyso PC does not interfere with the NF-kB mediated responses in DCs. As a result, hyperactive DCs share similar properties with active DCs treated with the PAMP R848 alone, but add IL-1beta to their cytokine secretion repertoire.

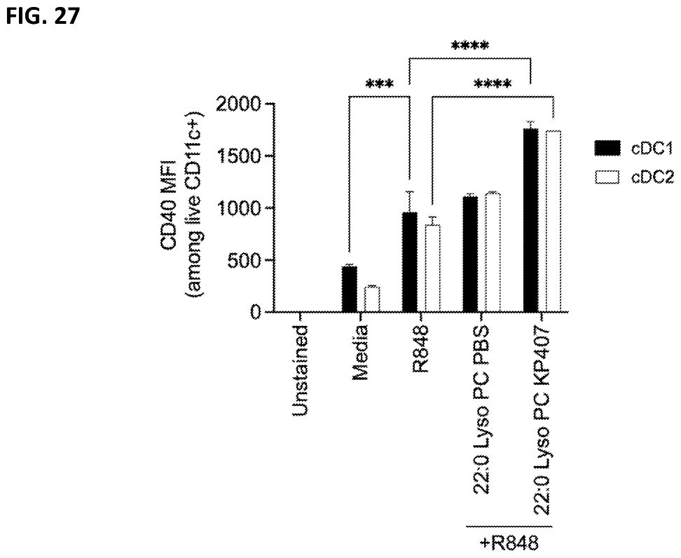

FLT3L-DCs are divided into two major subsets: cDC1s and cDC2s. Of these subsets, cDC1s are uniquely capable of antigen cross-presentation and can prime naive CD8+ T cells, but also CD4+ T cells. In contrast, cDC2s activate Th2 and Th17 immunity. To determine the behavior of 22:0 Lyso PC in cDC1 and cDC2 subsets, we analyzed cDC1s and cDC2s from FLT3L-differentiated DCs by flow cytometry. To identify cDC1 and cDC2, FLT3L DCs were stained with CD11c, SIRPa, CD24, MHC-II and CD45R. cDC1 are defined as CD11c+MHC-II+CD45R−CD24+SIRPa−, whereas cDC2 are defined as CD11c+MHC-II+CD45R−CD24lowSIRPa+. We analyzed the expression of co-stimulatory molecules such as CD40, which is crucial for DC interaction with T cells. As expected, 24 hours post stimulation, R848 induced the upregulation of CD40 expression as compared to naïve cDC1 and cDC2 cells. Interestingly, we found that CD40 expression was strongly enhanced in hyperactive cDC1 and cDC2 that were treated with R848 in combination with 41 uM 22:0 LPC resuspended in 5% KP407 (FIG. 27).

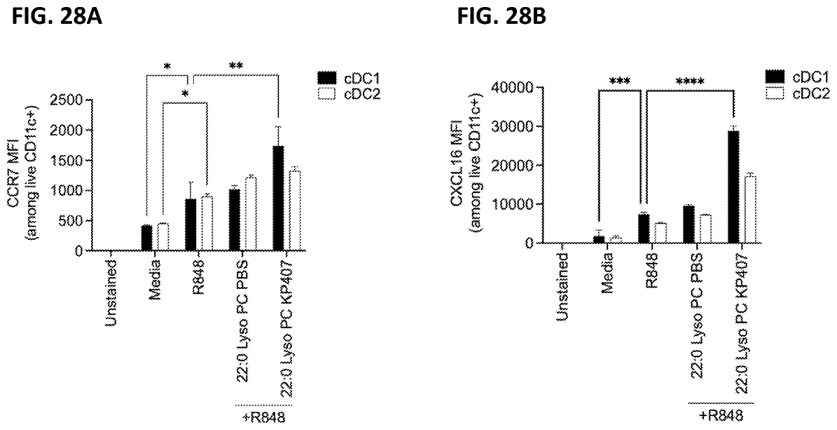

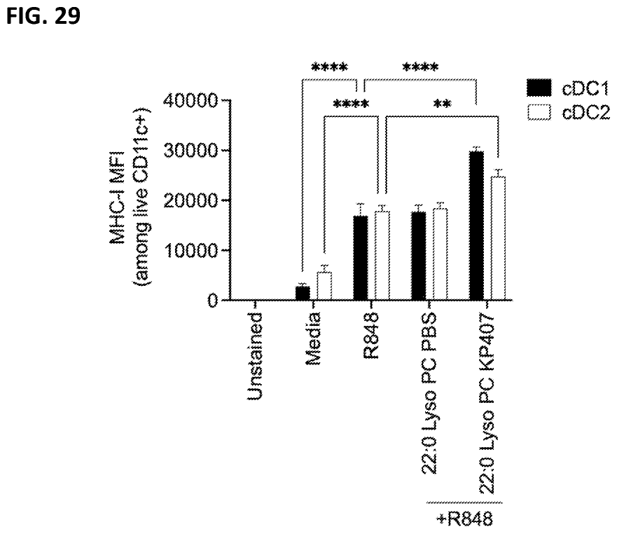

DC migration is a critical step in activating an adaptive immune response. CCR7 is a chemokine receptor required for DC migration to lymph nodes. When cells were hyperactivated using 22:0 Lyso PC in the KP407 formulation, cDC1 cells increased their CCR7 expression significantly compared to R848 alone (FIG. 28A). A similar trend was observed for CXCL16, a chemoattractant for T cells that plays a key role for anti-tumor T cell interaction with DCs in the tumor microenvironment (FIG. 28B). Additionally, hyperactivation enhanced MHC class I expression on both cDC1 and cDC2 subsets compared to R848 stimulation alone (FIG. 29).

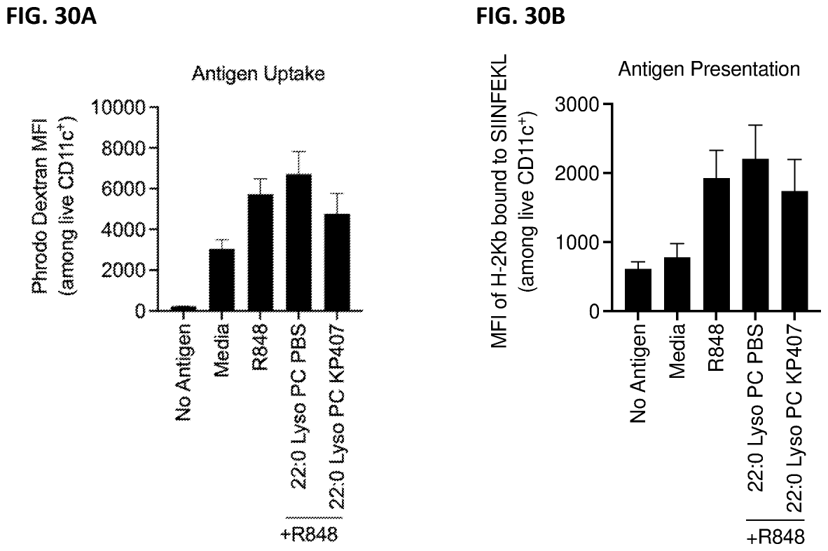

MHC class I (MHC-I) expression on DCs is important for antigen cross-presentation to CD8+ T cells. Therefore, we analyzed the ability of DCs to uptake and cross-present antigens on MHC-I, which is a DC function that is necessary for the stimulation of antigen-specific T cells. To first test DC endocytic capability, FLT3L-DCs were stimulated as described above for 24 hours and then incubated with Red pHrodo dextran. When dextran is endocytosed, red fluorescence can be detected by flow cytometry. DCs that did not receive dextran served as a negative control. We found, that when DC are treated with R848 in the presence or absence of 22:0 Lyso PC, DCs uptake antigens similarly, indicating that 22:0 Lyso PC does not compromise the antigen uptake capacity of DCs (FIG. 30A). To test antigen processing and antigen cross-presentation, DCs were stimulated as above for 24 hours, then treated with ovalbumin whole protein for 45 minutes at 4° C. or 37° C. We then measured the expression of ovalbumin peptides on MHC-I molecule by using an antibody specific for H-2Kb-bound to SIINFEKL. We found that DCs treated with R848 strongly enhance their ability to process and cross-present OVA antigen. Notably, 22:0 Lyso PC did not interfere with these functions since hyperactive DCs were still able to process antigen and cross-present OVA peptides on MHC-I molecules (FIG. 30B). Collectively, these data indicate that hyperactive DCs are potent at inducing adaptive T cell immune responses.

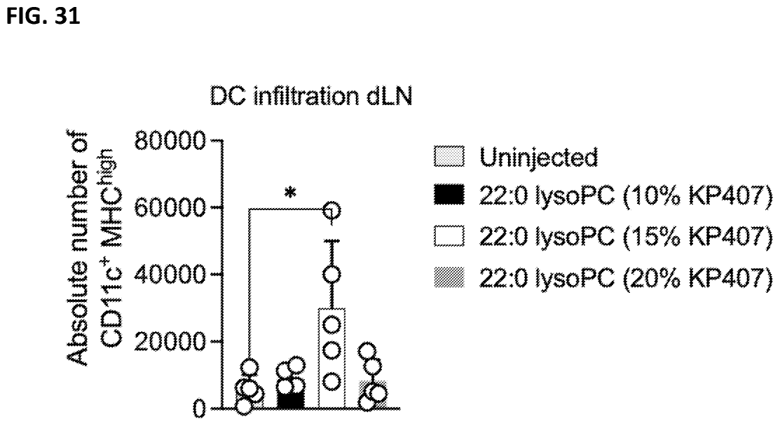

We envision that 22:0 Lyso PC in combination with R848 can mount an anti-tumor immune response utilizing endogenous DCs. As observed in vitro, micellization of 22:0 Lyso PC using KP407 is necessary to hyperactivate DC. To optimize the amount of KP407 required for in vivo vaccinations of mice, varying percentages were used in subcutaneous injections. Mice were subcutaneously immunized with R848 in combination with 22:0 Lyso PC that was resuspended in KP407 at 10%, 15%, or 20%. When 15% KP407 was used, we observed the highest influx of DCs defined as CD11c+MHChigh in the dLN, indicating that KP407 at 15% is the optimal concentration to induce DC trafficking to the lymph nodes (FIG. 31). This initial proof of concept study helped optimize the vaccination protocol for subsequent work.

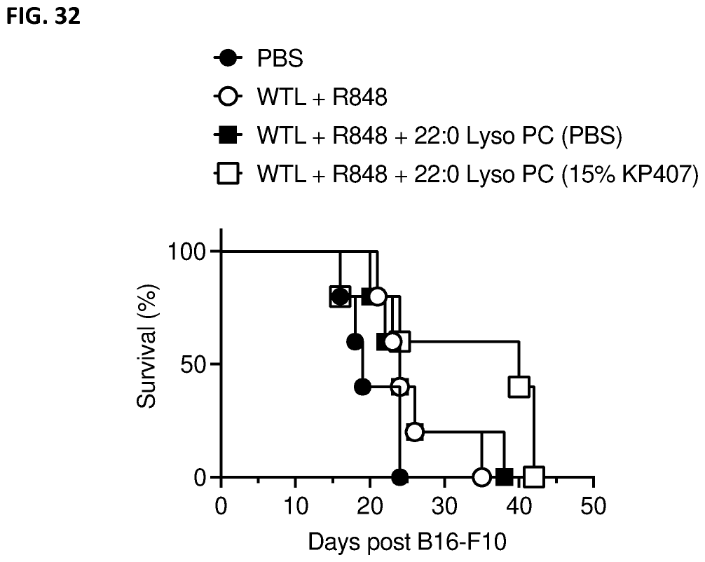

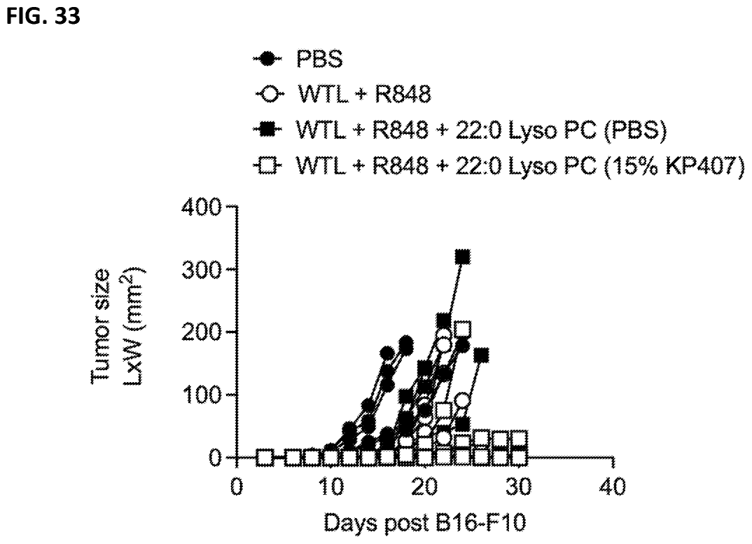

22:0 Lyso PC was then tested as a therapeutic tumor vaccine. Mice were inoculated subcutaneously on the left flank with B16-F10, a mouse melanoma tumor cell line. As a source of vaccine antigen, B16-F10 cell-derived whole tumor lysates (WTL) were used following 3-4 freeze/thaw cycles to release antigen from the tumor cells. To treat tumor-bearing mice, 7 days post tumor injection mice were immunized on the right flank with 80 ug of WTL in combination with 100 ug per mouse of R848, and 65 ug of 22:0 Lyso PC in KP407 (15%). Mice then received 2 boosts every 7 days. Unimmunized mice succumbed to tumor growth within 3 weeks of tumor inoculation. No significant benefit was observed when mice were vaccinated with tumor lysate combined with R848 or tumor lysate combine with R848 and 22:0 Lyso PC in PBS. In contrast, mice immunized with WTL and R848 in combination with 22:0 Lyso PC formulated with 15% KP407 survived longer (FIG. 32) and had delayed tumor growth kinetics (FIG. 33). This initial study indicates that 22:0 Lyso PC has hyperactivating effects on mouse DCs and can initiate a protective immune response against tumor challenge.

We claim:

1. A composition comprising an isolated lysophosphatidylcholine (LPC) with a single acyl chain, and a TLR7/8 agonist, wherein the acyl chain is a C13-C24 acyl chain.

2. The composition of claim 1, wherein the acyl chain is a C18-C22 acyl chain or a C21-C24 acyl chain.

3. The composition of claim 1 or claim 2, further comprising an antigen.

4. The composition of any one of claims 1-3, further comprising dendritic cells.

5. A composition comprising an isolated lysophosphatidylcholine (LPC) with a single acyl chain, and an antigen, wherein the acyl chain is a C21-C24 acyl chain.

6. The composition of claim 5, further comprising dendritic cells.

7. The composition of claim 5 or claim 6, further comprising a TLR7/8 agonist.

8. A composition comprising an isolated lysophosphatidylcholine (LPC) with a single acyl chain, and dendritic cells, wherein the acyl chain is a C21-C24 acyl chain.

9. The composition of claim 8, further comprising a TLR7/8 agonist.

10. The composition of claim 8 or claim 9, further comprising an antigen.

11. A composition of any one of claims 1-10, wherein the acyl chain is a C22 acyl chain.

12. The composition of any one of claims 1-11, wherein the acyl chain is fully saturated.

13. The composition of any one of claims 1-12, wherein the LPC comprises 1-behenoyl-2-hydroxy-sn-glycero-3-phosphocholine [LPC(22:0)].

14. The composition of any one of claims 1-13, wherein the TLR7/8 agonist is a small molecule with a molecule weight of 900 daltons or less.

15. The composition of claim 14, wherein the TLR7/8 agonist comprises an imidazoquinoline compound.

16. The composition of claim 15, wherein the TLR7/8 agonist comprises resiquimod (R848).

17. The composition of claim 14 or claim 15, wherein the TLR7/8 agonist does not inhibit NLR family pyrin domain containing 3 (NLRP3).

18. The composition of claim 13, wherein the LPC comprises LPC(22:0), and the TLR7/8 agonist comprises resiquimod (R848).

19. The composition of any one of claims 1-18, wherein the antigen is present in a biological sample obtained from an individual.

20. The composition of claim 19, wherein the biological sample comprises biopsy tissue.

21. The composition of claim 19, wherein the biological sample comprises cells.

22. The composition of claim 19, wherein the biological sample does not comprise cells.

23. The composition of claim 19, wherein the biological sample comprises pus from an abscess.

24. The composition of any one of claims 1-23, wherein the antigen comprises a proteinaceous antigen.

25. The composition of claim 24, wherein the antigen comprises a tumor antigen.

26. The composition of claim 25, wherein the tumor antigen comprises a synthetic or recombinant neoantigen.

27. The composition of claim 26, wherein the tumor antigen comprises a tumor cell lysate.

28. The composition of claim 24, wherein the antigen comprises a microbial antigen and the microbial antigen comprises one or more of a viral antigen, a bacterial antigen, a protozoan antigen, and a fungal antigen.

29. The composition of claim 28, wherein the microbial antigen comprises a purified or recombinant surface protein.

30. The composition of claim 28, wherein the microbial antigen comprises an inactivated, whole virus.

31. The composition of any one of claims 1-30, wherein the composition does not comprise liposomes.

32. The composition of any one of claims 1-31, wherein the composition does not comprise LPS or MPLA.

33. The composition of any one of claims 1-32, wherein the composition does not comprise oxPAPC or a species of oxPAPC.

34. The composition of claim 33, wherein the composition does not comprise HOdiA-PC, KOdiA-PC, HOOA-PC, KOOA-PC, and/or PGPC.

35. The composition of any one of claims 1-34, further comprising an adjuvant, wherein the adjuvant comprises an aluminum salt adjuvant, a squalene-in-water emulsion, a saponin, or combinations thereof.

36. A pharmaceutical formulation comprising the composition of any one of claims 1-35 and a pharmaceutically acceptable excipient.

37. A method for production of hyperactivated dendritic cells, the method comprising contacting the dendritic cells with a composition comprising effective amounts of an isolated lysophosphatidylcholine (LPC) with a single C13-C24 acyl chain, and a TLR7/8 agonist for production of hyperactivated dendritic cells, wherein the hyperactivated dendritic cells secrete IL-1beta without undergoing pyroptosis.

38. The method of claim 37, wherein the dendritic cells are contacted ex vivo with the composition of any one of claims 1-35 or the formulation of claim 36.

39. The method of claim 37, wherein the dendritic cells are contacted in vivo with the formulation of claim 36.

40. A pharmaceutical formulation comprising at least 10³, 10⁴, 10⁵ or 10⁶ of the hyperactivated dendritic cells produced by the method of claim 38, and a pharmaceutically acceptable excipient.

41. A method of stimulating an immune response against an antigen, comprising administering an effective amount of the formulation of claim 36 to an individual in need thereof to stimulate the immune response against the antigen.

42. A method of treating cancer, comprising administering an effective amount of the formulation of claim 36 to an individual in need thereof to treat the cancer.

43. A method of inhibiting abnormal cell proliferation, comprising administering an effective amount of the formulation of claim 36 to an individual in need thereof to inhibit abnormal cell proliferation.

44. A method of treating an infectious disease, comprising administering an effective amount of the formulation of claim 36 to an individual in need thereof to treat the infectious disease.

45. Use of the formulation of claim 36 for inducing an immune response against the antigen in an individual in need thereof.

46. Use of the formulation of claim 36 for inducing an anti-tumor immune response in an individual in need thereof, wherein the individual is or was tumor-bearing.

47. Use of the formulation of claim 36 for inducing an anti-microbe immune response in an individual in need thereof, wherein the individual is infected with the microbe or has not been exposed to the microbe.

48. The composition, formulation, method or use of any one of claims 19-47, wherein the individual is a mammalian subject.

49. The composition, formulation, method or use of any one of claims 19-47, wherein the individual is a human subject.

50. A method of preparing an immunogenic composition, the method comprising:
a) depleting leukocytes from a suspension of cells prepared from a tumor to obtain a tumor cell-enriched suspension;
b) lysing cells from the tumor cell-enriched suspension to obtain a tumor cell lysate; and
c) contacting the tumor cell lysate with an isolated lysophosphatidylcholine (LPC) having a single acyl chain and a toll-like receptor 7/8 (TLR7/8) agonist to obtain the immunogenic composition, wherein the acyl chain is a C13-C24 acyl chain.

51. The method of claim 50, wherein the leukocytes are depleted in step a) by negative selection using an anti-CD45 antibody.

52. The method of claim 50 or claim 51, wherein the cells are lysed in step b) by one or more freeze-thaw cycles.

53. The method of any one of claims 50-52, wherein the acyl chain is a fully saturated C18-C22 acyl chain or a fully saturated C18-C24 acyl chain.

54. The method of claim 53, wherein the LPC comprises 1-behenoyl-2-hydroxy-sn-glycero-3-phosphocholine [LPC(22:0)].

55. The method of any one of claims 50-54, wherein the TLR7/8 agonist is a small molecule with a molecule weight of 900 daltons or less.

56. The method of claim 55, wherein the TLR7/8 agonist comprises an imidazoquinoline compound.

57. The method of claim 56, wherein the TLR7/8 agonist comprises resiquimod (R848).

58. The method of claim 55 or claim 56, wherein the TLR7/8 agonist does not inhibit NLR family pyrin domain containing 3 (NLRP3).

59. The method of claim 54, wherein the LPC comprises LPC(22:0), and the TLR7/8 agonist comprises resiquimod (R848).

60. The method of any one of claims 50-59, further comprising before step a) obtaining a sample from the tumor from a mammalian subject with cancer and preparing the suspension of cells from the sample.

61. An immunogenic composition prepared by the method of any one of claims 50-60.

62. A method of eliciting an anti-cancer immune response, the method comprising:
administering to a mammalian subject with cancer an effective amount of the immunogenic composition of claim 61.

63. The method of claim 62, wherein the anti-cancer immune response comprises cellular immune response.

64. The method of claim 63, wherein the anti-cancer immune response comprises cancer antigen-induced IL-1beta secretion and/or activation of CD8+T lymphocytes.

65. The method of any one of claims 62-64, wherein the cancer is a non-hematologic cancer.

66. The method of claim 65, wherein the non-hematologic cancer is a carcinoma, a sarcoma, or a melanoma.

67. The method of any one of claims 62-64, wherein the cancer is a lymphoma.

68. A method of treating cancer, the method comprising:
a) preparing an immunogenic composition comprising a tumor cell lysate, an isolated lysophosphatidylcholine (LPC) having a single acyl chain, and a toll-like receptor 7/8 (TLR7/8) agonist, wherein the tumor cell lysate is or has been prepared from a sample of a tumor obtained from the mammalian subject with cancer, and the acyl chain is a C13-C24 acyl chain; and
b) administering to the subject an effective amount of the immunogenic composition.

69. The method of any one of claims 62-68, wherein the acyl chain is a fully saturated C18-C22 acyl chain or a fully saturated C18-C24 acyl chain.

70. The method of claim 68, wherein the LPC comprises 1-behenoyl-2-hydroxy-sn-glycero-3-phosphocholine [LPC(22:0)].

71. The method of any one of claims 62-70, wherein the TLR7/8 agonist is a small molecule with a molecule weight of 900 daltons or less.

72. The method of claim 71, wherein the TLR7/8 agonist comprises an imidazoquinoline compound.

73. The method of claim 72, wherein the TLR7/8 agonist comprises resiquimod (R848).

74. The method of claim 70, wherein the LPC comprises 22:0 LPC, and the TLR7/8 agonist comprises resiquimod (R848).

75. The method of any one of claims 68-74, further comprising administering to the subject an effective amount of an additional therapeutic agent.

76. The method of claim 75, wherein the additional therapeutic agent comprises one or more of the group consisting of an immune checkpoint inhibitor, an antineoplastic agent, and radiation therapy.

77. A composition comprising an isolated lysophosphatidylcholine (LPC) with a single acyl chain, and a pathogen recognition receptor (PRR) agonist, wherein the acyl chain is a C13-C24 acyl chain.

78. The composition of claim 77, wherein the PRR agonist is an agonist of a toll-like receptor (TLR), a NOD-like receptor (NLR), a RIG-I-like receptor (RLR), or a C-type lectin receptor (CLR).

79. The composition of claim 77, wherein the PRR agonist is an agonist of a cytosolic DNA sensor (CDS) or a stimulator of IFN genes (STING).

80. The composition of claim 77, wherein the PRR agonist comprises one or more of R848, TL8-506, LPS, Pam2CSK4, and ODN 2336.

81. The composition of any one of claims 77-80, further comprising an antigen.

82. The composition of any one of claims 77-81, further comprising dendritic cells.

83. A pharmaceutical formulation comprising the composition of any one of claims 77-82 and a pharmaceutically acceptable excipient.

84. A pharmaceutical formulation comprising an isolated lysophosphatidylcholine (LPC) with a single acyl chain, and a pharmaceutically acceptable excipient, wherein the acyl chain is a C21-C24 acyl chain.

85. The pharmaceutical formulation of claim 83 or claim 84, wherein the acyl chain is a fully saturated C22 acyl chain.

86. The pharmaceutical formulation of claim 85, wherein the LPC comprises 1-behenoyl-2-hydroxy-sn-glycero-3-phosphocholine [LPC(22:0)].

87. A composition for hyperactivation of human dendritic cells, comprising an isolated lysophosphatidylcholine (LPC) with a single acyl chain, and a pathogen recognition receptor (PRR) agonist, wherein the acyl chain is C22 acyl chain, and wherein the composition is effective for achieving a higher level of dendritic cell hyperactivation than a comparator composition comprising PGPC in place of the LPC.

88. The composition of claim 87, wherein the higher level of dendritic cell hyperactivation comprises induction of IL-1beta secretion from the human dendritic cells in vitro at a level that is at least 2, 3 or 4 fold higher when contacted with the composition comprising the LPC and the PRR agonist than when contacted with the comparator composition comprising the PGPC and the PRR agonist, wherein the PRR agonist is LPS.

89. The composition of claim 88, wherein the concentration of the LPC and the concentration of the PGPC are the same concentration in a range of from about 10 μM to about 80 μM, and the LPS is present at a concentration of 1 μg/ml in both the composition and the comparator composition.

90. The composition of claim 88, wherein the higher level of dendritic cell hyperactivation comprises a lipid activity index for IL-1beta secretion from the human dendritic cells for the composition comprising the LPC and the PRR agonist that is at least 4, 5 or 6 fold higher in activity units than that of the comparator composition comprising the LPC and the PRR agonist.

91. The composition, formulation, method or use of any one of claims 19-47, wherein the individual is a canine subject.

92. The composition, formulation, method or use of any one of claims 60-90, wherein the mammalian subject is a human patient.

93. The composition, formulation, method or use of any one of claims 60-90, wherein the mammalian subject is a non-human patient.

94. The composition, formulation, method or use of any one of claims 60-90, wherein the mammalian subject is a canine patient.

95. The composition, formulation, method or use of any one of claim 1-90 or 92, wherein the dendritic cells are human dendritic cells.

96. The composition, formulation, method or use of any one of claim 1-91 or 94, wherein the dendritic cells are canine dendritic cells.

97. The composition, method or use of claim 95 or claim 96, wherein the dendritic cells are present in a composition comprising peripheral blood mononuclear cells (PBMCs).

98. The composition, method or use of any one of claims 37-49 or claim 91, wherein the hyperactivated dendritic cells secrete one or both of IFNγ and TNFα.

99. The composition, formulation, method or use of any one of claims 1-98, comprising a surfactant.

100. The composition, formulation, method or use of claim 99, wherein the surfactant comprises a non-ionic surfactant.

101. The composition, formulation, method or use of claim 100, wherein the non-ionic surfactant comprises an ethylene oxide-propylene oxide copolymer.

102. The composition, formulation, method or use of claim 100, wherein the non-ionic surfactant comprises one or more of Poloxamer 407, Poloxamer 188, and P123.

103. The composition, formulation, method or use of claim 100, wherein the non-ionic surfactant comprises Poloxamer 407.

104. The composition, formulation, method or use of any one of claims 100-103, wherein i) the LPC is dissolved in an alcohol to form an LPC alcohol solution; ii) the LPC alcohol solution is mixed with the non-ionic surfactant to form a mixture; and iii) the alcohol is evaporated from the mixture to form particles comprising the LPC and the non-ionic surfactant.

105. The composition, formulation, method or use of any one of claims 100-104, wherein the non-ionic surfactant is present in an amount of about 2.5% to 25% (w/w), optionally about 5% to 20% (w/w), optionally about 15% (w/w).

106. The composition, formulation, method or use of any one of claims 100-105, wherein the LPC and non-ionic surfactant are present in particles with a diameter of about 1000 to 2000 nanometers, optionally with a diameter of about 1500 nanometers.
